SEQUENCE LISTING (8 sequences)

SEQ ID NO: 1 - DNA, 20 bp, synthetic construct
  source 1..20: mol_type = other DNA
aaattttggggaccaggaac

SEQ ID NO: 2 - DNA, 20 bp, synthetic construct
  source 1..20: mol_type = other DNA
tggcacctgtgtaggtcaac

SEQ ID NO: 3 - DNA, 29882 bp, Coronavirus 2
  source 1..29882: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaa

SEQ ID NO: 4 - DNA, 11354 bp, Coronavirus 2
  source 1..11354: mol_type = other DNA
aaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgttttgcagccgatcatcagcacatctaggttttgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgggcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcttggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttttaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagttaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaagtctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaactagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctatatcttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgattttgcaacatcagcttgtgttttggctgctgaatgtacaatttttaaagatgcttctggtaagccattaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaatctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccgctgtagcttattttaatatggtctatatgcctgctagttgggtgatgcgtattatgacatggttggatatggttgatactagtttgtctggttttaagctaaaagactgtgttatgtatgcatcagctgtggtgttactaatccttatgacagcaagaactgtgtatgatgatggtgctaggagag

SEQ ID NO: 5 - DNA, 9864 bp, Coronavirus 2
  source 1..9864: mol_type = other DNA
tcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacttacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcagttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaactacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgttctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaacccagcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagcttatgtagaatggaagttctatgatgcacagccttgtagtgacaaagcttataaaatagaagaattattctattcttatgccacacattctgacaaattcacagatggtgtatgcctattttggaattgcaatgtcgatagatatcctgttaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttagaaccagaactcaattaccccctgcatacactaattctttcacacgtggtgtttattaccctgacaaagttttcagatcctcagttttacattcaactcaggacttgttcttacct

SEQ ID NO: 6 - DNA, 269 bp, Coronavirus 2
  source 1..269: mol_type = other DNA
ttattaccacaaaaacaacaaaagttggatggaaagtggagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaaca

SEQ ID NO: 7 - DNA, 7380 bp, Coronavirus 2
  source 1..7380: mol_type = other DNA
agagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagattgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcgtcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaaaatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactcctttagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattattttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgttttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctaccgcaatggcttgtcttgtaggcttgatgtggctcagctacttcattgcttctttcagactgtttgcgcgtacgcgttccatgtggtcattcaatccagaaactaatattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagcttcacctaaactgttcatcagacaagaggaagttcaagaactttactctccaatttttcttattgttgcggcaatagtgtttataacactttgcttcacactcaaaagaaagatagaatgattgaactttcattaattgacttctatttgtgctttttagcctttctgctattccttgttttaattatgcttattatcttttggttctcacttgaactgcaagatcataatgaaatttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttaatctaaacgaacaaactaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaaggccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtatgggaacttctcctgctagaatggctggcaatggctgtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggcttatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccactcggagtacgatcgagtgtacagtgaacaatgctagggagagctgcctatatggaagagccctaatgtgtaaaattaattttagtagtgctatccccatgtgattttaatagctnnnnnnnnnnnnnnnaaaaaaaaaaaaaaaaaaaaaaaaaaaaaaaaa

SEQ ID NO: 8 - DNA, 29804 bp, Coronavirus 2
  source 1..29804: mol_type = other DNA
aacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggttttgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaggtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttttaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagcggctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatataaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaagtcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattactttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttatacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaatctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcacaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtgttgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagtcacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacttacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaatatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacgtacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgttatctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttggaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattatagtgcgtgagccagaagatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttgatgaagtttttaacgccaccagatttgcatctgtttatgcttggaacaggaagagaatcagcaactgtgttgctgattattctgtcctatataatctcgcaccatttttcacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaagcttgattctaaggttagtggtaattataattacctgtatagattgtttaggaagtctaatctcaaaccttttgagagagatatttcaactgaaatctatcaggccggtaacaaaccttgtaatggtgttgcaggttttaattgttactttcctttacgatcatatagtttccgacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaaaaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaatatgtcaacaactcatatgagtgtgacatacccattggtgcaggtatatgcgctagttatcagactcagactaagtctcatcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaacgtgctttaactggaatagctgttgaacaagacaaaaacacccaagaagtttttgcacaagtcaaacaaatttacaaaacaccaccaattaaatattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaaaggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccataatgcacaagctttaaacacgcttgttaaacaacttagctccaaatttggtgcaatttcaagtgttttaaatgatatcttttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagattcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataactctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagataggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcaggttccaacggtactattaccgttgaagagcttaaaaagctccttgaagaatggaacctagtaataggtttcctattccttacatggatttgtcttctacaatttgcctatgccaacaggaataggtttttgtatataattaagttaattttcctctggctgttatggccagtaactttaacttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgcggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgttattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaacttaaatgtctgataatggaccccaaaatcagcgaaatgcactccgcattacgtttggtggaccctcagattcaactggcagtaaccagaatggtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaaacgaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttctt

(19) United States
(12) Patent Application Publication
(10) Pub. No.: US 2024/0209381 A1
(43) Pub. Date: Jun. 27, 2024

(54) RAPID GENERATION OF INFECTIOUS CLONES

(71) Applicant: The J. David Gladstone Institutes, San Francisco, CA (US)

(72) Inventors: Taha Yasin Taha, Walnut Creek, CA (US); Melanie Ott, Mill Valley, CA (US); Takako Tabata, San Francisco, CA (US)

(21) Appl. No.: 18/393,522

(22) Filed: Dec. 21, 2023

Related U.S. Application Data
(60) Provisional application No. 63/434,828, filed on Dec. 22, 2022.

(51) Int. Cl. C12N 15/66

(52) U.S. Cl. CPC C12N 15/66; C12N 2770/20043

(57) ABSTRACT

Provided herein is a novel cloning system with rational fragment design and single-pot ligation (pGLUE) that allows systematic exchange and mutagenesis of genes and rapid construction of entire molecular clones and replicons of virus within days.

### PRIORITY

This application claims the benefit of priority to U.S. Provisional Appln Ser. No. 63/434,828, filed Dec. 22, 2022, which is incorporated by reference herein as if fully set forth herein.

### INCORPORATION BY REFERENCE OF SEQUENCE LISTING

This application contains a Sequence Listing which has been submitted electronically in ST26 format and is hereby incorporated by reference in its entirety. Said ST26 file, created on Dec. 19, 2023, is named “2394842.xml” and is 98,304 bytes in size.

### BACKGROUND

The COVID-19 pandemic continues to be a major threat to public health despite the development of vaccines and therapeutics. This is due to the emergence of variants with enhanced pathogenesis and/or immune evasion.

### SUMMARY

To access variants rapidly and to conduct investigations into the contribution of mutations to viral fitness and/or immune escape, it is necessary to construct infectious clones. The invention described herein can be used as a tool to rapidly generate virus infectious clones, such as positive and negative strand RNA viruses, retroviruses, including, but not limited to, SARS-COV-2, common cold coronavirus (such as HKU1), respiratory syncytial virus (RSV) and variant/mutants thereof, that can be utilized to study emerging variants of concern. As new variants are identified, it typically takes weeks before the virus is collected from patients and reliably propagated. The invention does not rely on patient isolates, but instead only requires the sequence of the variant. Consequently, the invention can generate viral variants rapidly to accelerate research studies and therefore improve the public health response to emerging variants. In addition to speed, the invention enables the construction of viruses lacking specific mutations to study the contribution of new mutations to viral fitness and/or immune escape.

One embodiment provides for a method for assembly of a recombinant viral genome from a plurality of DNA segments, comprising: a) preparing a series of partially overlapping viral DNA segments designed from a viral genome sequence, wherein each segment comprises different sequences from the viral genome, wherein said overlap comprises unique sequences on their 5′ and 3′ ends; b) cloning each of said viral DNA segments of a) into a plasmid, said plasmid comprising a cloning site that is flanked on both sides by a Type IIS restriction endonuclease recognition site or adapters are added to the 5′ and 3′ ends of each viral DNA segment prior to cloning in a plasmid, wherein the adapters comprise the recognition site for a Type IIS restriction endonuclease, said sites positioned to allow removal by digestion with a Type IIS enzyme of a defined number of bases from one strand on both ends of the viral DNA segment; c) validating the cloned insert segment in each clone of b); d) digesting the clones of c) with the Type IIS restriction enzyme, releasing the cloned insert DNA segments, now modified by removal of the defined number of bases from at least one strand at each terminus; and e) annealing and ligating in a single pot into a destination plasmid, whereby an assembled recombinant viral genome with a desired order and orientation of the cloned DNA segments is formed.

In one embodiment, the viral genome is SARS-COV-2, a variant of SARS-COV-2, or combination thereof. In one embodiment, the variant is a naturally occurring variant or genetically/recombinantly engineered variant. In one embodiment, the naturally occurring variant is WA1, Delta, or Omicron. In another embodiment, the virus is a common cold virus (such as HKU1), In one embodiment, the virus a negative strand virus, such as respiratory syncytial virus (RSV).

In one embodiment, the insert DNA segments that are ligated together in e) come from a single viral variant. In another embodiment, the insert DNA segments that are ligated together in e) come from more than one viral variant. In one embodiment, a complete viral genome is formed from the ligated insert DNA segments of e). In another embodiment, the insert DNA segments are ligated together in e), one or more viral ORFs are absent. In one embodiment, the absent ORF is the ORF coding for S, N, M or E viral proteins. In one embodiment, the absent ORF codes for the S protein. In one embodiment, a mutation has wherein a mutation has been entered into one of the viral DNA segments of a). In one embodiment, the mutation is single point mutation, an addition or a deletion of a nucleotide or an amino acid.

In one embodiment, the viral genome is divided into a plurality of DNA segments, wherein there are at least 2 segments. In embodiment, the viral genome is divided into a plurality of DNA segments, wherein there are 2, 3, 4, 5, 6, 7, 8, 9, 10, 11, 12, 13, 14, 15, 16, 17, 18, 19, 20 or more segments. In another embodiment, the viral genome is divided into a plurality of DNA segments, wherein there are 8 to 12 segments. In one embodiment, the viral genome is divided into a plurality of DNA segments, wherein there are 10 segments.

In embodiment, each of the viral DNA segments of b) are flanked by a Type IIS restriction endonuclease restriction site with opposite orientation. In another embodiment, the cloning plasmid comprises a cloning site that is flanked on both sides by a Type IIS restriction endonuclease recognition site. In one embodiment, the Type IIS restriction endonuclease comprises one or more of BbsI, BbvI, BcoDI, BfuAI, BsaI, BsmAI, BsmFI, BspMI, BtgZI, Esp3I, FokI, PaqCI, SfaNI, BacI, and HgaI. In one embodiment, the Type IIS restriction endonuclease is BsaI.

In one embodiment, the insert is validated in c) by means of sequencing or mapping. In one embodiment, the insert DNA segments are ligated with a DNA ligase. In one embodiment, the destination plasmid is pBAC. In one embodiment, the destination plasmid comprises at least one promotor and Type IIS restriction endonuclease sites.

In one embodiment, the assembled recombinant viral genome of e) is transfected into cells for production of virus. In one embodiment, the virus is infectious. In another embodiment, the assembled recombinant viral genome is subjected to in vitro transcription with T7 polymerase so as to yield RNA. In one embodiment, the RNA is electroporated into cells and virus is produced.

One embodiment provides a kit for use in a method for assembly of a recombinant viral genome from a plurality of viral DNA segments to form at least one recombinant viral genome, the kit comprising a plurality of viral DNA segments or instructions on how to produce a plurality of viral DNA segments, which at least one of each of the plurality of viral DNA segments can be assembled with another of the plurality DNA segments, a cloning plasmid and wherein the plurality of viral DNA molecules are flanked in each case by a Type IIS restriction endonuclease restriction site with opposite orientation or wherein the cloning plasmid comprises a cloning site that is flanked on both sides by a Type IIS restriction endonuclease recognition site. In one embodiment, the kit further comprises a Type IIS restriction endonuclease and a DNA ligase.

### BRIEF DESCRIPTION OF THE DRAWINGS

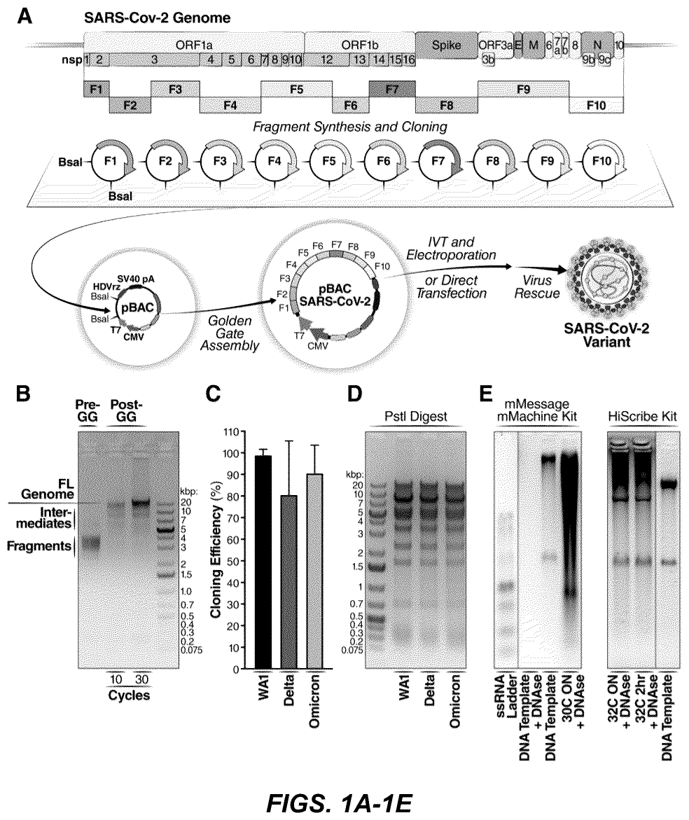

FIGS. 1A-1E. Golden Gate assembly enables rapid cloning of SARS-COV-2 variants.
(A) Schematic of cloning methodology and generation of infectious clones. The viral genome was rationally divided into 10 fragments and assembled into a BAC vector containing T7 and CMV promoters, HDVrz, and SV40 polyA sequence. The assembled vector was then directly transfected into cells or first in vitro transcribed into RNA, followed by electroporation into cells to generate SARS-COV-2 variants.
(B) Agarose gel electrophoresis of Golden Gate (GG) assembly of the 10 fragments.
(C) Cloning efficiency of SARS-COV-2 variant infectious clones. Correct colonies are defined as those with perfectly correct sequence across the entire genome. 20-40 colonies were analyzed for each variant.
(D) Agarose gel electrophoresis of PstI digest of 0.5 μg of SARS-COV-2 variant infectious clone plasmids, demonstrating high quantity and quality of plasmid preps.
(E) In vitro transcription of assembled plasmid to generate full-length RNA under different conditions with two different commercial kits.

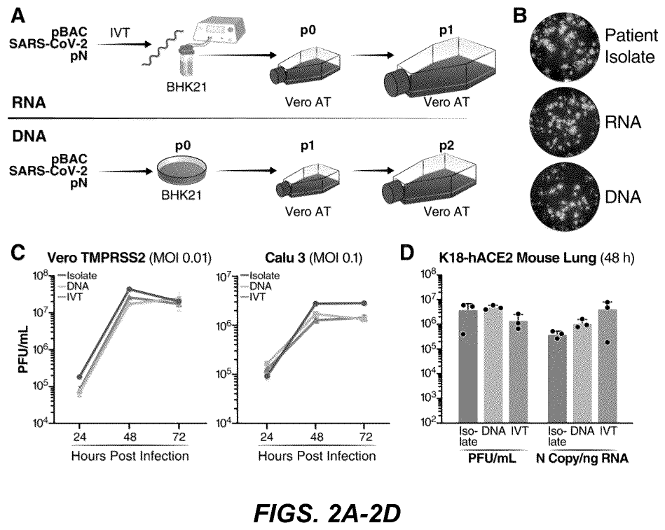

FIGS. 2A-2D. DNA- and RNA-launched viruses replicate similarly to virus derived from patient isolates.
(A) Schematic of virus rescue from RNA or DNA. For RNA-launched virus rescue, in vitro transcribed RNA from viral construct and N expression construct is electroporated into BHK-21 cells followed by co-culture with Vero ACE2 TMPRSS2 cells to yield p0 viral stock and propagated in the same cells onward. For DNA-launched virus rescue, viral construct and N expression construct are directly transfected into BHK-21 cells to yield p0 viral stock, which is then propagated in Vero ACE2 TMPRSS2 cells.
(B) Plaque morphology of DNA- and RNA-launched and patient-derived Delta variant viruses. Images were pseudocolored to black and white for optimal visualization. The images represent at least three independent replicates.
(C) Growth kinetics of the viruses in B in Vero TMPRSS2 and Calu3 cells over 72 hours as measured by infectious particle release by plaque assay. Average of three independent experiments analyzed in duplicate±SD are shown.
(D) Replication of the viruses in B was assessed in K18-hACE2 mice lungs at 48 hours post-infection by infectious particle release by plaque assay and viral RNA by RT-qPCR. Average of three independent experiments analyzed in duplicate±SD are shown.

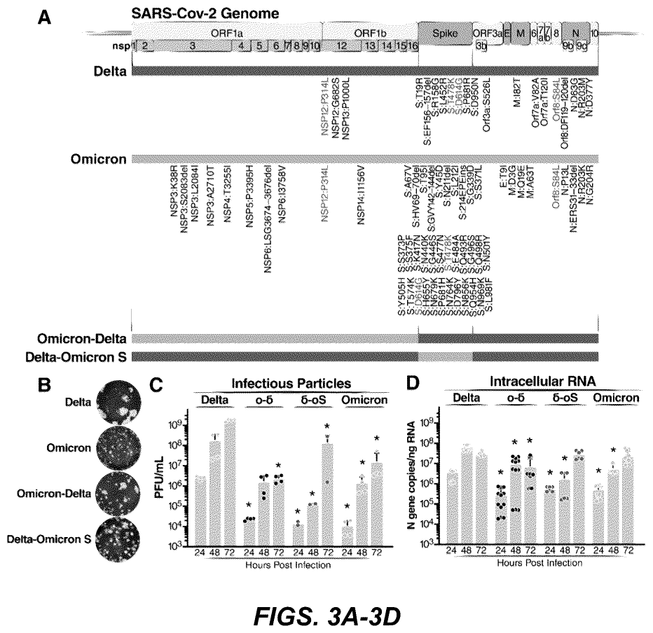

FIGS. 3A-3D. Omicron mutations in Spike and ORF1ab reduce viral particle production and intracellular RNA levels.
(A) Schematic of recombinant infectious clones of Delta (black) and Omicron (yellow) variants with indicated mutations. Mutations represent >90% of GISAID sequences of each variant as of January 2022.
(B) Representative images of plaques from indicated recombinant infectious clones. Images were pseudocolored to black and white for optimal visualization.
(C) Extracellular infectious particles from infected Calu3 cells (m.o.i. 0.1). Average of three independent experiments analyzed in duplicate±SD are shown and compared to Delta by two-sided Student's T-test at each timepoint.
(D) Intracellular RNA was quantified from infected Calu3 cells (m.o.i. of 0.1). Data are expressed in absolute copies/μg based on a standard curve of N gene with known copy number. Average of three independent experiments analyzed in duplicate±SD are shown and compared to Delta by two-sided Student's T-test at each timepoint. *, p<0.01.

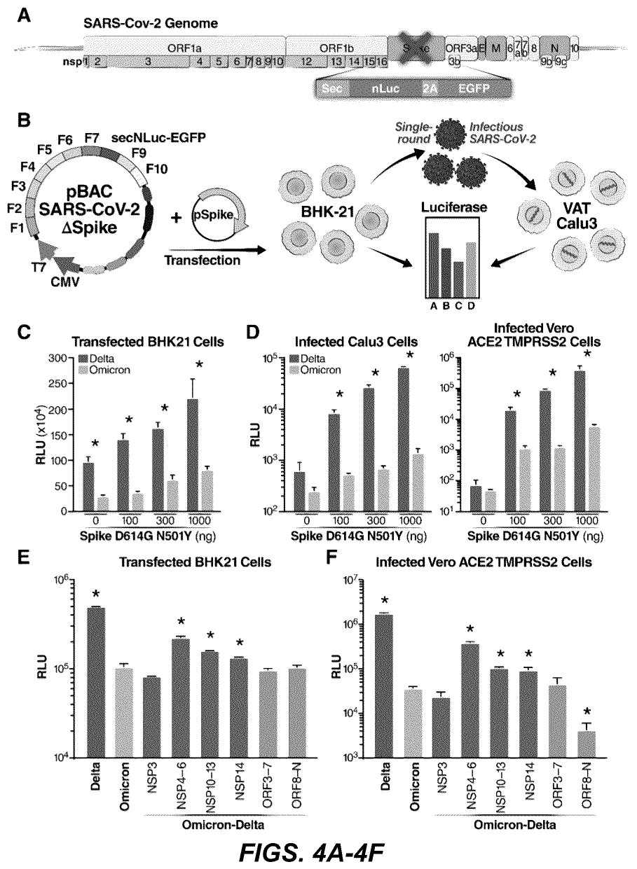

FIGS. 4A-4F. Omicron mutations attenuate viral replication independent of spike.
(A) Schematic of the replicon system in which the Spike gene was replaced with secreted luciferase (Sec: secretion signal, nLuc: Nano luciferase, eGFP: enhanced green fluorescent protein).
(B) Experimental workflow of the SARS-COV-2 replicon assay. VAT, Vero cells stably overexpressing ACE2 and TMPRSS2.
(C) Luciferase readout from cells transfected with increasing amounts of Spike expression construct paired with either the Delta or Omicron replicon plasmids. Average of two independent experiments analyzed in duplicate±SD and pairwise comparisons between the Delta and Omicron variants by two-sided Student's T-test are shown.
(D) Luciferase readout from Calu3 or Vero-ACE2/TMPRSS2 cells infected with supernatant from BHK21 cells transfected with Delta or Omicron replicons in B. Shown are the average of two independent experiments analyzed in duplicate±SD and pairwise comparisons between the Delta and Omicron variants by two-sided Student's T-test.
(E) Luciferase readout from transfected BHK21 cells with Omicron-Delta recombinant replicons launched with Delta Spike as indicated. Shown are the average of two independent experiments analyzed in triplicate±SD, and comparisons were made relative to the Omicron variant by two-sided Student's T-test.
(F) Luciferase readout from infected Vero ACE2 TMPRSS2 cells infected with supernatant from E. Average of two independent experiments analyzed in triplicate±SD are shown, and comparisons were made relative to the Omicron variant by two-sided Student's T-test.

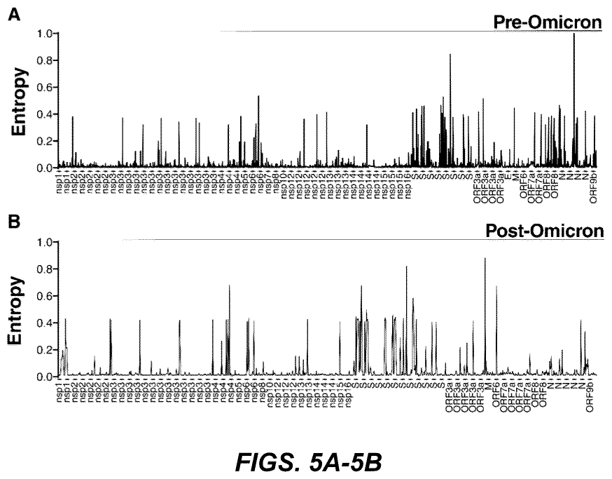

FIGS. 5A-5B. Entropy analysis reveals mutational hotspots across the SARS-CoV-2 genome.
(A) Entropy analysis of subsampled SARS-CoV-2 sequences pre-Omicron emergence (December 2019-November 2021). Data were adapted from Nextstrain GISAID global analysis as of Aug. 19, 2022 (51) and normalized Shannon entropy values per amino acid.
(B) Entropy analysis of subsampled SARS-CoV-2 sequences post-Omicron emergence (January 2022-August 2022). Data were adapted from Nextstrain GISAID global analysis as of Aug. 19, 2022 (51) and normalized Shannon entropy values per amino acid.

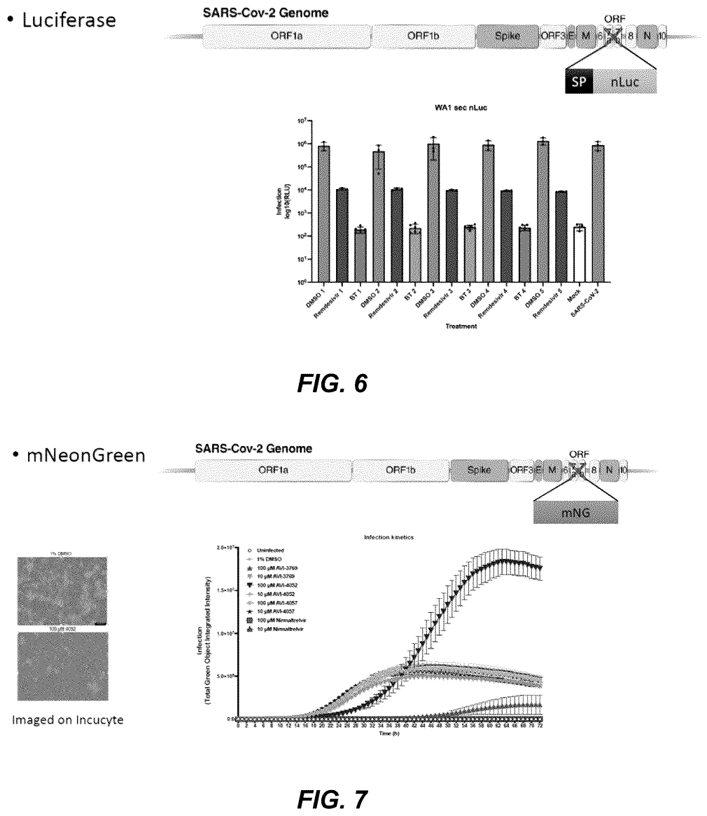

FIG. 6. Generation of SARS-CoV-2 luciferase reporter virus for antiviral testing.
A luciferase reporter SARS-CoV-2 was generated by cloning in a secreted nanoluciferase protein in place of Orf7a and Orf7b. The virus was rescued as described in FIG. 2A and validated for antiviral testing using the approved antiviral remdesivir. BT: bleed-through luciferase signal to neighbor wells.

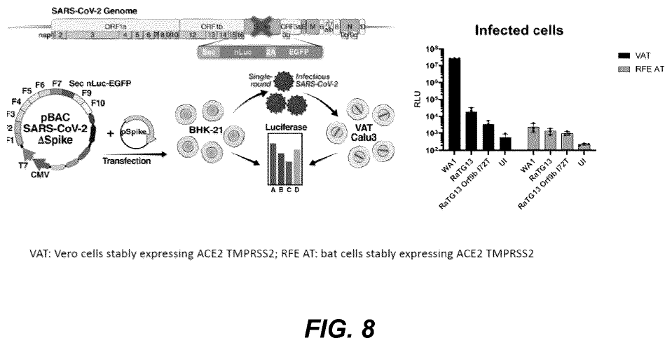

FIG. 7. Generation of SARS-CoV-2 fluorescence reporter virus for antiviral testing.
A fluorescence reporter SARS-CoV-2 was generated by cloning in mNeonGreen protein in place of Orf7a and Orf7b. The virus was rescued as described in FIG. 2A and validated for antiviral testing using the approved antiviral nirmatrelvir and other investigational antivirals. The panel on the left shows fluorescence intensity for the DMSO and antiviral treated cells. The panel on the right shows the live cell imaging fluorescence intensity over 72 hours post-infection.

FIG. 8. Generation of RaTG13 virus and comparison of replication capacity to SARS-CoV-2.
A Spike replicon of the bat SARS-related coronavirus RaTG13 and a mutant RaTG13 Orf9b I72T were constructed similar to the SARS-CoV-2 replicon in FIG. 4A. Single-round infectious particles were generated and used to infect Vero cells or RFE (bat) cells stably expressing ACE2 and TMPRSS2 (VAT and RFE AT, respectively). The left panel shows schematic of the experiment and the right panel shows luciferase levels measured 72 hours post-infection.

### DESCRIPTION OF THE INVENTION

Current methods to construct SARS-CoV-2 infectious clones are laborious and therefore have limited accessibility by most labs. It also requires several weeks to clone and assemble the infectious clone, which can be a barrier to investigate emerging variants in a timely manner. The presently described invention overcomes these issues by decreasing the time needed to construct infectious clones to 1-2 weeks and increasing the quality of the method by producing a clonal population of virus that can be sequence verified prior to conducting experiments.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, including at least about 75%, at least about 90%, or at least about 95%, or at least about 97% of the nucleotide residues of the first portion are capable of base pairing with nucleotide residues in the second portion. In some embodiments, all nucleotide residues of the first portion are capable of base pairing with nucleotide residues in the second portion.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

“Coding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene codes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as coding the protein or other product of that gene or cDNA.

As used herein, an “essentially pure” preparation of a particular DNA or protein is a preparation wherein at least about 90%, at least about 95%, such as at least about 99%, by weight, of the DNA protein in the preparation.

A “fragment” or “segment” is a portion of a longer DNA sequence comprising at least one nucleotide. The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC5′ share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques like those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

“Recombinant polynucleotide” or “recombinant vial genome” refers to a polynucleotide having sequences that have been joined together in vitro. An assembled recombinant polynucleotide may be included in a suitable vector, and the vector can be used to transform a suitable host cell. A recombinant polynucleotide may serve or include a non-coding function (e.g., promoter, origin of replication, ribosome-binding site, termination, polyA etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “vector” or “plasmid” is a composition of matter which comprises an isolated nucleic acid and which can be used to deliver the isolated nucleic acid to the interior of a cell. Vectors and plasmids can also be called “expression vector” or “expression plasmid” which refer to a vector comprising a recombinant polynucleotide comprising expression control sequences (e.g., one or more polymers) operatively linked to a nucleotide sequence to be expressed. An expression vector comprises sufficient cis-acting elements for expression; other elements for expression can be supplied by the host cell or in an in vitro expression system (promoters, polyA sites, termination). Expression vectors include all those known in the art, such as cosmids, plasmids (e.g., naked or contained in liposomes) and pBAC.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

### Viruses

The invention is a method to rapidly clone viral genomes, such as common cold viruses, (e.g., HKU1), positive or negative strand RNA viruses (including RaTG13 and respiratory syncytial virus (RSV)) or SARS-CoV-2 and variants thereof, such as Omicron, Delta and others (including mutations/variants thereof made in a laboratory setting; the invention also includes the use/study of other coronaviruses, as well as RNA viruses in general, and the methods can be applied to some DNA viruses as well), without the need for laborious cloning strategies that can limit accessibility.

```
WA1: Genbank MN985325.1
Nucleic Acid Sequence
(SEQ ID NO: 3)
        1 attaaaggtt tataccttcc caggtaacaa accaaccaac tttcgatctc ttgtagatct
      61 gttctctaaa cgaactttaa aatctgtgtg gctgtcactc ggctgcatgc ttagtgcact
    121 cacgcagtat aattaataac taattactgt cgttgacagg acacgagtaa ctcgtctatc
    181 ttctgcaggc tgcttacggt ttcgtccgtg ttgcagccga tcatcagcac atctaggttt
    241 cgtccgggtg tgaccgaaag gtaagatgga gagccttgtc cctggtttca acgagaaaac
    301 acacgtccaa ctcagtttgc ctgttttaca ggttcgcgac gtgctcgtac gtggctttgg
    361 agactccgtg gaggaggtct tatcagaggc acgtcaacat cttaaagatg gcacttgtgg
    421 cttagtagaa gttgaaaaag gcgttttgcc tcaacttgaa cagccctatg tgttcatcaa
    481 acgttcggat gctcgaactg cacctcatgg tcatgttatg gttgagctgg tagcagaact
    541 cgaaggcatt cagtacggtc gtagtggtga gacacttggt gtccttgtcc ctcatgtggg
    601 cgaaatacca gtggcttacc gcaaggttct tcttcgtaag aacggtaata aaggagctgg
    661 tggccatagt tacggcgccg atctaaagtc atttgactta ggcgacgagc ttggcactga
    721 tccttatgaa gattttcaag aaaactggaa cactaaacat agcagtggtg ttacccgtga
    781 actcatgcgt gagcttaacg gaggggcata cactcgctat gtcgataaca acttctgtgg
    841 ccctgatggc taccctcttg agtgcattaa agaccttcta gcacgtgctg gtaaagcttc
    901 atgcactttg tccgaacaac tggactttat tgacactaag aggggtgtat actgctgccg
    961 tgaacatgag catgaaattg cttggtacac ggaacgttct gaaaagagct atgaattgca
  1021 gacacctttt gaaattaaat tggcaaagaa atttgacacc ttcaatgggg aatgtccaaa
  1081 ttttgtattt cccttaaatt ccataatcaa gactattcaa ccaagggttg aaaagaaaaa
  1141 gcttgatggc tttatgggta gaattcgatc tgtctatcca gttgcgtcac caaatgaatg
  1201 caaccaaatg tgcctttcaa ctctcatgaa gtgtgatcat tgtggtgaaa cttcatggca
  1261 gacgggcgat tttgttaaag ccacttgcga attttgtggc actgagaatt tgactaaaga
  1321 aggtgccact acttgtggtt acttacccca aaatgctgtt gttaaaattt attgtccagc
  1381 atgtcacaat tcagaagtag gacctgagca tagtcttgcc gaataccata atgaatctgg
  1441 cttgaaaacc attcttcgta agggtggtcg cactattgcc tttggaggct gtgtgttctc
  1501 ttatgttggt tgccataaca agtgtgccta ttgggttcca cgtgctagcg ctaacatagg
  1561 ttgtaaccat acaggtgttg ttggagaagg ttccgaaggt cttaatgaca accttcttga
  1621 aatactccaa aaagagaaag tcaacatcaa tattgttggt gactttaaac ttaatgaaga
  1681 gatcgccatt attttggcat ctttttctgc ttccacaagt gcttttgtgg aaactgtgaa
  1741 aggtttggat tataaagcat tcaaacaaat tgttgaatcc tgtggtaatt ttaaagttac
  1801 aaaaggaaaa gctaaaaaag gtgcctggaa tattggtgaa cagaaatcaa tactgagtcc
  1861 tctttatgca tttgcatcag aggctgctcg tgttgtacga tcaattttct cccgcactct
  1921 tgaaactgct caaaattctg tgcgtgtttt acagaaggcc gctataacaa tactagatgg
  1981 aatttcacag tattcactga gactcattga tgctatgatg ttcacatctg atttggctac
  2041 taacaatcta gttgtaatgg cctacattac aggtggtgtt gttcagttga cttcgcagtg
  2101 gctaactaac atctttggca ctgtttatga aaaactcaaa cccgtccttg attggcttga
  2161 agagaagttt aaggaaggtg tagagtttct tagagacggt tgggaaattg ttaaatttat
  2221 ctcaacctgt gcttgtgaaa ttgtcggtgg acaaattgtc acctgtgcaa aggaaattaa
  2281 ggagagtgtt cagacattct ttaagcttgt aaataaattt ttggctttgt gtgctgactc
  2341 tatcattatt ggtggagcta aacttaaagc cttgaattta ggtgaaacat ttgtcacgca
  2401 ctcaaaggga ttgtacagaa agtgtgttaa atccagagaa gaaactggcc tactcatgcc
  2461 tctaaaagcc ccaaaagaaa ttatcttctt agagggagaa acacttccca cagaagtgtt
  2521 aacagaggaa gttgtcttga aaactggtga tttacaacca ttagaacaac ctactagtga
  2581 agctgttgaa gctccattgg ttggtacacc agtttgtatt aacgggctta tgttgctcga
  2641 aatcaaagac acagaaaagt actgtgccct tgcacctaat atgatggtaa caaacaatac
  2701 cttcacactc aaaggcggtg caccaacaaa ggttactttt ggtgatgaca ctgtgataga
  2761 agtgcaaggt tacaagagtg tgaatatcac ttttgaactt gatgaaagga ttgataaagt
  2821 acttaatgag aagtgctctg cctatacagt tgaactcggt acagaagtaa atgagttcgc
  2881 ctgtgttgtg gcagatgctg tcataaaaac tttgcaacca gtatctgaat tacttacacc
  2941 actgggcatt gatttagatg agtggagtat ggctacatac tacttatttg atgagtctgg
  3001 tgagtttaaa ttggcttcac atatgtattg ttctttctac cctccagatg aggatgaaga
  3061 agaaggtgat tgtgaagaag aagagtttga gccatcaact caatatgagt atggtactga
  3121 agatgattac caaggtaaac ctttggaatt tggtgccact tctgctgctc ttcaacctga
  3181 agaagagcaa gaagaagatt ggttagatga tgatagtcaa caaactgttg gtcaacaaga
  3241 cggcagtgag gacaatcaga caactactat tcaaacaatt gttgaggttc aacctcaatt
  3301 agagatggaa cttacaccag ttgttcagac tattgaagtg aatagtttta gtggttattt
  3361 aaaacttact gacaatgtat acattaaaaa tgcagacatt gtggaagaag ctaaaaaggt
  3421 aaaaccaaca gtggttgtta atgcagccaa tgtttacctt aaacatggag gaggtgttgc
  3481 aggagcctta aataaggcta ctaacaatgc catgcaagtt gaatctgatg attacatagc
  3541 tactaatgga ccacttaaag tgggtggtag ttgtgtttta agcggacaca atcttgctaa
  3601 acactgtctt catgttgtcg gcccaaatgt taacaaaggt gaagacattc aacttcttaa
  3661 gagtgcttat gaaaatttta atcagcacga agttctactt gcaccattat tatcagctgg
  3721 tatttttggt gctgacccta tacattcttt aagagtttgt gtagatactg ttcgcacaaa
  3781 tgtctactta gctgtctttg ataaaaatct ctatgacaaa cttgtttcaa gctttttgga
  3841 aatgaagagt gaaaagcaag ttgaacaaaa gatcgctgag attcctaaag aggaagttaa
  3901 gccatttata actgaaagta aaccttcagt tgaacagaga aaacaagatg ataagaaaat
  3961 caaagcttgt gttgaagaag ttacaacaac tctggaagaa actaagttcc tcacagaaaa
  4021 cttgttactt tatattgaca ttaatggcaa tcttcatcca gattctgcca ctcttgttag
  4081 tgacattgac atcactttct taaagaaaga tgctccatat atagtgggtg atgttgttca
  4141 agagggtgtt ttaactgctg tggttatacc tactaaaaag gctggtggca ctactgaaat
  4201 gctagcgaaa gctttgagaa aagtgccaac agacaattat ataaccactt acccgggtca
  4261 gggtttaaat ggttacactg tagaggaggc aaagacagtg cttaaaaagt gtaaaagtgc
  4321 cttttacatt ctaccatcta ttatctctaa tgagaagcaa gaaattcttg gaactgtttc
  4381 ttggaatttg cgagaaatgc ttgcacatgc agaagaaaca cgcaaattaa tgcctgtctg
  4441 tgtggaaact aaagccatag tttcaactat acagcgtaaa tataagggta ttaaaataca
  4501 agagggtgtg gttgattatg gtgctagatt ttacttttac accagtaaaa caactgtagc
  4561 gtcacttatc aacacactta acgatctaaa tgaaactctt gttacaatgc cacttggcta
  4621 tgtaacacat ggcttaaatt tggaagaagc tgctcggtat atgagatctc tcaaagtgcc
  4681 agctacagtt tctgtttctt cacctgatgc tgttacagcg tataatggtt atcttacttc
  4741 ttcttctaaa acacctgaag aacattttat tgaaaccatc tcacttgctg gttcctataa
  4801 agattggtcc tattctggac aatctacaca actaggtata gaatttctta agagaggtga
  4861 taaaagtgta tattacacta gtaatcctac cacattccac ctagatggtg aagttatcac
  4921 ctttgacaat cttaagacac ttctttcttt gagagaagtg aggactatta aggtgtttac
  4981 aacagtagac aacattaacc tccacacgca agttgtggac atgtcaatga catatggaca
  5041 acagtttggt ccaacttatt tggatggagc tgatgttact aaaataaaac ctcataattc
  5101 acatgaaggt aaaacatttt atgttttacc taatgatgac actctacgtg ttgaggcttt
  5161 tgagtactac cacacaactg atcctagttt tctgggtagg tacatgtcag cattaaatca
  5221 cactaaaaag tggaaatacc cacaagttaa tggtttaact tctattaaat gggcagataa
  5281 caactgttat cttgccactg cattgttaac actccaacaa atagagttga agtttaatcc
  5341 acctgctcta caagatgctt attacagagc aagggctggt gaagctgcta acttttgtgc
  5401 acttatctta gcctactgta ataagacagt aggtgagtta ggtgatgtta gagaaacaat
  5461 gagttacttg tttcaacatg ccaatttaga ttcttgcaaa agagtcttga acgtggtgtg
  5521 taaaacttgt ggacaacagc agacaaccct taagggtgta gaagctgtta tgtacatggg
  5581 cacactttct tatgaacaat ttaagaaagg tgttcagata ccttgtacgt gtggtaaaca
  5641 agctacaaaa tatctagtac aacaggagtc accttttgtt atgatgtcag caccacctgc
  5701 tcagtatgaa cttaagcatg gtacatttac ttgtgctagt gagtacactg gtaattacca
  5761 gtgtggtcac tataaacata taacttctaa agaaactttg tattgcatag acggtgcttt
  5821 acttacaaag tcctcagaat acaaaggtcc tattacggat gttttctaca aagaaaacag
  5881 ttacacaaca accataaaac cagttactta taaattggat ggtgttgttt gtacagaaat
  5941 tgaccctaag ttggacaatt attataagaa agacaattct tatttcacag agcaaccaat
  6001 tgatcttgta ccaaaccaac catatccaaa cgcaagcttc gataatttta agtttgtatg
  6061 tgataatatc aaatttgctg atgatttaaa ccagttaact ggttataaga aacctgcttc
  6121 aagagagctt aaagttacat ttttccctga cttaaatggt gatgtggtgg ctattgatta
  6181 taaacactac acaccctctt ttaagaaagg agctaaattg ttacataaac ctattgtttg
  6241 gcatgttaac aatgcaacta ataaagccac gtataaacca aatacctggt gtatacgttg
  6301 tctttggagc acaaaaccag ttgaaacatc aaattcgttt gatgtactga agtcagagga
  6361 cgcgcaggga atggataatc ttgcctgcga agatctaaaa ccagtctctg aagaagtagt
  6421 ggaaaatcct accatacaga aagacgttct tgagtgtaat gtgaaaacta ccgaagttgt
  6481 aggagacatt atacttaaac cagcaaataa tagtttaaaa attacagaag aggttggcca
  6541 cacagatcta atggctgctt atgtagacaa ttctagtctt actattaaga aacctaatga
  6601 attatctaga gtattaggtt tgaaaaccct tgctactcat ggtttagctg ctgttaatag
  6661 tgtcccttgg gatactatag ctaattatgc taagcctttt cttaacaaag ttgttagtac
  6721 aactactaac atagttacac ggtgtttaaa ccgtgtttgt actaattata tgccttattt
  6781 ctttacttta ttgctacaat tgtgtacttt tactagaagt acaaattcta gaattaaagc
  6841 atctatgccg actactatag caaagaatac tgttaagagt gtcggtaaat tttgtctaga
  6901 ggcttcattt aattatttga agtcacctaa tttttctaaa ctgataaata ttataatttg
  6961 gtttttacta ttaagtgttt gcctaggttc tttaatctac tcaaccgctg ctttaggtgt
  7021 tttaatgtct aatttaggca tgccttctta ctgtactggt tacagagaag gctatttgaa
  7081 ctctactaat gtcactattg caacctactg tactggttct ataccttgta gtgtttgtct
  7141 tagtggttta gattctttag acacctatcc ttctttagaa actatacaaa ttaccatttc
  7201 atcttttaaa tgggatttaa ctgcttttgg cttagttgca gagtggtttt tggcatatat
  7261 tcttttcact aggtttttct atgtacttgg attggctgca atcatgcaat tgtttttcag
  7321 ctattttgca gtacatttta ttagtaattc ttggcttatg tggttaataa ttaatcttgt
  7381 acaaatggcc ccgatttcag ctatggttag aatgtacatc ttctttgcat cattttatta
  7441 tgtatggaaa agttatgtgc atgttgtaga cggttgtaat tcatcaactt gtatgatgtg
  7501 ttacaaacgt aatagagcaa caagagtcga atgtacaact attgttaatg gtgttagaag
  7561 gtccttttat gtctatgcta atggaggtaa aggcttttgc aaactacaca attggaattg
  7621 tgttaattgt gatacattct gtgctggtag tacatttatt agtgatgaag ttgcgagaga
  7681 cttgtcacta cagtttaaaa gaccaataaa tcctactgac cagtcttctt acatcgttga
  7741 tagtgttaca gtgaagaatg gttccatcca tctttacttt gataaagctg gtcaaaagac
  7801 ttatgaaaga cattctctct ctcattttgt taacttagac aacctgagag ctaataacac
  7861 taaaggttca ttgcctatta atgttatagt ttttgatggt aaatcaaaat gtgaagaatc
  7921 atctgcaaaa tcagcgtctg tttactacag tcagcttatg tgtcaaccta tactgttact
  7981 agatcaggca ttagtgtctg atgttggtga tagtgcggaa gttgcagtta aaatgtttga
  8041 tgcttacgtt aatacgtttt catcaacttt taacgtacca atggaaaaac tcaaaacact
  8101 agttgcaact gcagaagctg aacttgcaaa gaatgtgtcc ttagacaatg tcttatctac
  8161 ttttatttca gcagctcggc aagggtttgt tgattcagat gtagaaacta aagatgttgt
  8221 tgaatgtctt aaattgtcac atcaatctga catagaagtt actggcgata gttgtaataa
  8281 ctatatgctc acctataaca aagttgaaaa catgacaccc cgtgaccttg gtgcttgtat
  8341 tgactgtagt gcgcgtcata ttaatgcgca ggtagcaaaa agtcacaaca ttgctttgat
  8401 atggaacgtt aaagatttca tgtcattgtc tgaacaacta cgaaaacaaa tacgtagtgc
  8461 tgctaaaaag aataacttac cttttaagtt gacatgtgca actactagac aagttgttaa
  8521 tgttgtaaca acaaagatag cacttaaggg tggtaaaatt gttaataatt ggttgaagca
  8581 gttaattaaa gttacacttg tgttcctttt tgttgctgct attttctatt taataacacc
  8641 tgttcatgtc atgtctaaac atactgactt ttcaagtgaa atcataggat acaaggctat
  8701 tgatggtggt gtcactcgtg acatagcatc tacagatact tgttttgcta acaaacatgc
  8761 tgattttgac acatggttta gtcagcgtgg tggtagttat actaatgaca aagcttgccc
  8821 attgattgct gcagtcataa caagagaagt gggttttgtc gtgcctggtt tgcctggcac
  8881 gatattacgc acaactaatg gtgacttttt gcatttctta cctagagttt ttagtgcagt
  8941 tggtaacatc tgttacacac catcaaaact tatagagtac actgactttg caacatcagc
  9001 ttgtgttttg gctgctgaat gtacaatttt taaagatgct tctggtaagc cagtaccata
  9061 ttgttatgat accaatgtac tagaaggttc tgttgcttat gaaagtttac gccctgacac
  9121 acgttatgtg ctcatggatg gctctattat tcaatttcct aacacctacc ttgaaggttc
  9181 tgttagagtg gtaacaactt ttgattctga gtactgtagg cacggcactt gtgaaagatc
  9241 agaagctggt gtttgtgtat ctactagtgg tagatgggta cttaacaatg attattacag
  9301 atctttacca ggagttttct gtggtgtaga tgctgtaaat ttacttacta atatgtttac
  9361 accactaatt caacctattg gtgctttgga catatcagca tctatagtag ctggtggtat
  9421 tgtagctatc gtagtaacat gccttgccta ctattttatg aggtttagaa gagcttttgg
  9481 tgaatacagt catgtagttg cctttaatac tttactattc cttatgtcat tcactgtact
  9541 ctgtttaaca ccagtttact cattcttacc tggtgtttat tctgttattt acttgtactt
  9601 gacattttat cttactaatg atgtttcttt tttagcacat attcagtgga tggttatgtt
  9661 cacaccttta gtacctttct ggataacaat tgcttatatc atttgtattt ccacaaagca
  9721 tttctattgg ttctttagta attacctaaa gagacgtgta gtctttaatg gtgtttcctt
  9781 tagtactttt gaagaagctg cgctgtgcac ctttttgtta aataaagaaa tgtatctaaa
  9841 gttgcgtagt gatgtgctat tacctcttac gcaatataat agatacttag ctctttataa
  9901 taagtacaag tattttagtg gagcaatgga tacaactagc tacagagaag ctgcttgttg
  9961 tcatctcgca aaggctctca atgacttcag taactcaggt tctgatgttc tttaccaacc
10021 accacaaacc tctatcacct cagctgtttt gcagagtggt tttagaaaaa tggcattccc
10081 atctggtaaa gttgagggtt gtatggtaca agtaacttgt ggtacaacta cacttaacgg
10141 tctttggctt gatgacgtag tttactgtcc aagacatgtg atctgcacct ctgaagacat
10201 gcttaaccct aattatgaag atttactcat tcgtaagtct aatcataatt tcttggtaca
10261 ggctggtaat gttcaactca gggttattgg acattctatg caaaattgtg tacttaagct
10321 taaggttgat acagccaatc ctaagacacc taagtataag tttgttcgca ttcaaccagg
10381 acagactttt tcagtgttag cttgttacaa tggttcacca tctggtgttt accaatgtgc
10441 tatgaggccc aatttcacta ttaagggttc attccttaat ggttcatgtg gtagtgttgg
10501 ttttaacata gattatgact gtgtctcttt ttgttacatg caccatatgg aattaccaac
10561 tggagttcat gctggcacag acttagaagg taacttttat ggaccttttg ttgacaggca
10621 aacagcacaa gcagctggta cggacacaac tattacagtt aatgttttag cttggttgta
10681 cgctgctgtt ataaatggag acaggtggtt tctcaatcga tttaccacaa ctcttaatga
10741 ctttaacctt gtggctatga agtacaatta tgaacctcta acacaagacc atgttgacat
10801 actaggacct ctttctgctc aaactggaat tgccgtttta gatatgtgtg cttcattaaa
10861 agaattactg caaaatggta tgaatggacg taccatattg ggtagtgctt tattagaaga
10921 tgaatttaca ccttttgatg ttgttagaca atgctcaggt gttactttcc aaagtgcagt
10981 gaaaagaaca atcaagggta cacaccactg gttgttactc acaattttga cttcactttt
11041 agttttagtc cagagtactc aatggtcttt gttctttttt ttgtatgaaa atgccttttt
11101 accttttgct atgggtatta ttgctatgtc tgcttttgca atgatgtttg tcaaacataa
11161 gcatgcattt ctctgtttgt ttttgttacc ttctcttgcc actgtagctt attttaatat
11221 ggtctatatg cctgctagtt gggtgatgcg tattatgaca tggttggata tggttgatac
11281 tagtttgtct ggttttaagc taaaagactg tgttatgtat gcatcagctg tagtgttact
11341 aatccttatg acagcaagaa ctgtgtatga tgatggtgct aggagagtgt ggacacttat
11401 gaatgtcttg acactcgttt ataaagttta ttatggtaat gctttagatc aagccatttc
11461 catgtgggct cttataatct ctgttacttc taactactca ggtgtagtta caactgtcat
11521 gtttttggcc agaggtattg tttttatgtg tgttgagtat tgccctattt tcttcataac
11581 tggtaataca cttcagtgta taatgctagt ttattgtttc ttaggctatt tttgtacttg
11641 ttactttggc ctcttttgtt tactcaaccg ctactttaga ctgactcttg gtgtttatga
11701 ttacttagtt tctacacagg agtttagata tatgaattca cagggactac tcccacccaa
11761 gaatagcata gatgccttca aactcaacat taaattgttg ggtgttggtg gcaaaccttg
11821 tatcaaagta gccactgtac agtctaaaat gtcagatgta aagtgcacat cagtagtctt
11881 actctcagtt ttgcaacaac tcagagtaga atcatcatct aaattgtggg ctcaatgtgt
11941 ccagttacac aatgacattc tcttagctaa agatactact gaagcctttg aaaaaatggt
12001 ttcactactt tctgttttgc tttccatgca gggtgctgta gacataaaca agctttgtga
12061 agaaatgctg gacaacaggg caaccttaca agctatagcc tcagagttta gttcccttcc
12121 atcatatgca gcttttgcta ctgctcaaga agcttatgag caggctgttg ctaatggtga
12181 ttctgaagtt gttcttaaaa agttgaagaa gtctttgaat gtggctaaat ctgaatttga
12241 ccgtgatgca gccatgcaac gtaagttgga aaagatggct gatcaagcta tgacccaaat
12301 gtataaacag gctagatctg aggacaagag ggcaaaagtt actagtgcta tgcagacaat
12361 gcttttcact atgcttagaa agttggataa tgatgcactc aacaacatta tcaacaatgc
12421 aagagatggt tgtgttccct tgaacataat acctcttaca acagcagcca aactaatggt
12481 tgtcatacca gactataaca catataaaaa tacgtgtgat ggtacaacat ttacttatgc
12541 atcagcattg tgggaaatcc aacaggttgt agatgcagat agtaaaattg ttcaacttag
12601 tgaaattagt atggacaatt cacctaattt agcatggcct cttattgtaa cagctttaag
12661 ggccaattct gctgtcaaat tacagaataa tgagcttagt cctgttgcac tacgacagat
12721 gtcttgtgct gccggtacta cacaaactgc ttgcactgat gacaatgcgt tagcttacta
12781 caacacaaca aagggaggta ggtttgtact tgcactgtta tccgatttac aggatttgaa
12841 atgggctaga ttccctaaga gtgatggaac tggtactatc tatacagaac tggaaccacc
12901 ttgtaggttt gttacagaca cacctaaagg tcctaaagtg aagtatttat actttattaa
12961 aggattaaac aacctaaata gaggtatggt acttggtagt ttagctgcca cagtacgtct
13021 acaagctggt aatgcaacag aagtgcctgc caattcaact gtattatctt tctgtgcttt
13081 tgctgtagat gctgctaaag cttacaaaga ttatctagct agtgggggac aaccaatcac
13141 taattgtgtt aagatgttgt gtacacacac tggtactggt caggcaataa cagttacacc
13201 ggaagccaat atggatcaag aatcctttgg tggtgcatcg tgttgtctgt actgccgttg
13261 ccacatagat catccaaatc ctaaaggatt ttgtgactta aaaggtaagt atgtacaaat
13321 acctacaact tgtgctaatg accctgtggg ttttacactt aaaaacacag tctgtaccgt
13381 ctgcggtatg tggaaaggtt atggctgtag ttgtgatcaa ctccgcgaac ccatgcttca
13441 gtcagctgat gcacaatcgt ttttaaacgg gtttgcggtg taagtgcagc ccgtcttaca
13501 ccgtgcggca caggcactag tactgatgtc gtatacaggg cttttgacat ctacaatgat
13561 aaagtagctg gttttgctaa attcctaaaa actaattgtt gtcgcttcca agaaaaggac
13621 gaagatgaca atttaattga ttcttacttt gtagttaaga gacacacttt ctctaactac
13681 caacatgaag aaacaattta taatttactt aaggattgtc cagctgttgc taaacatgac
13741 ttctttaagt ttagaataga cggtgacatg gtaccacata tatcacgtca acgtcttact
13801 aaatacacaa tggcagacct cgtctatgct ttaaggcatt ttgatgaagg taattgtgac
13861 acattaaaag aaatacttgt cacatacaat tgttgtgatg atgattattt caataaaaag
13921 gactggtatg attttgtaga aaacccagat atattacgcg tatacgccaa cttaggtgaa
13981 cgtgtacgcc aagctttgtt aaaaacagta caattctgtg atgccatgcg aaatgctggt
14041 attgttggtg tactgacatt agataatcaa gatctcaatg gtaactggta tgatttcggt
14101 gatttcatac aaaccacgcc aggtagtgga gttcctgttg tagattctta ttattcattg
14161 ttaatgccta tattaacctt gaccagggct ttaactgcag agtcacatgt tgacactgac
14221 ttaacaaagc cttacattaa gtgggatttg ttaaaatatg acttcacgga agagaggtta
14281 aaactctttg accgttattt taaatattgg gatcagacat accacccaaa ttgtgttaac
14341 tgtttggatg acagatgcat tctgcattgt gcaaacttta atgttttatt ctctacagtg
14401 ttcccaccta caagttttgg accactagtg agaaaaatat ttgttgatgg tgttccattt
14461 gtagtttcaa ctggatacca cttcagagag ctaggtgttg tacataatca ggatgtaaac
14521 ttacatagct ctagacttag ttttaaggaa ttacttgtgt atgctgctga ccctgctatg
14581 cacgctgctt ctggtaatct attactagat aaacgcacta cgtgcttttc agtagctgca
14641 cttactaaca atgttgcttt tcaaactgtc aaacccggta attttaacaa agacttctat
14701 gactttgctg tgtctaaggg tttctttaag gaaggaagtt ctgttgaatt aaaacacttc
14761 ttctttgctc aggatggtaa tgctgctatc agcgattatg actactatcg ttataatcta
14821 ccaacaatgt gtgatatcag acaactacta tttgtagttg aagttgttga taagtacttt
14881 gattgttacg atggtggctg tattaatgct aaccaagtca tcgtcaacaa cctagacaaa
14941 tcagctggtt ttccatttaa taaatggggt aaggctagac tttattatga ttcaatgagt
15001 tatgaggatc aagatgcact tttcgcatat acaaaacgta atgtcatccc tactataact
15061 caaatgaatc ttaagtatgc cattagtgca aagaatagag ctcgcaccgt agctggtgtc
15121 tctatctgta gtactatgac caatagacag tttcatcaaa aattattgaa atcaatagcc
15181 gccactagag gagctactgt agtaattgga acaagcaaat tctatggtgg ttggcacaac
15241 atgttaaaaa ctgtttatag tgatgtagaa aaccctcacc ttatgggttg ggattatcct
15301 aaatgtgata gagccatgcc taacatgctt agaattatgg cctcacttgt tcttgctcgc
15361 aaacatacaa cgtgttgtag cttgtcacac cgtttctata gattagctaa tgagtgtgct
15421 caagtattga gtgaaatggt catgtgtggc ggttcactat atgttaaacc aggtggaacc
15481 tcatcaggag atgccacaac tgcttatgct aatagtgttt ttaacatttg tcaagctgtc
15541 acggccaatg ttaatgcact tttatctact gatggtaaca aaattgccga taagtatgtc
15601 cgcaatttac aacacagact ttatgagtgt ctctatagaa atagagatgt tgacacagac
15661 tttgtgaatg agttttacgc atatttgcgt aaacatttct caatgatgat actctctgac
15721 gatgctgttg tgtgtttcaa tagcacttat gcatctcaag gtctagtggc tagcataaag
15781 aactttaagt cagttcttta ttatcaaaac aatgttttta tgtctgaagc aaaatgttgg
15841 actgagactg accttactaa aggacctcat gaattttgct ctcaacatac aatgctagtt
15901 aaacagggtg atgattatgt gtaccttcct tacccagatc catcaagaat cctaggggcc
15961 ggctgttttg tagatgatat cgtaaaaaca gatggtacac ttatgattga acggttcgtg
16021 tctttagcta tagatgctta cccacttact aaacatccta atcaggagta tgctgatgtc
16081 tttcatttgt acttacaata cataagaaag ctacatgatg agttaacagg acacatgtta
16141 gacatgtatt ctgttatgct tactaatgat aacacttcaa ggtattggga acctgagttt
16201 tatgaggcta tgtacacacc gcatacagtc ttacaggctg ttggggcttg tgttctttgc
16261 aattcacaga cttcattaag atgtggtgct tgcatacgta gaccattctt atgttgtaaa
16321 tgctgttacg accatgtcat atcaacatca cataaattag tcttgtctgt taatccgtat
16381 gtttgcaatg ctccaggttg tgatgtcaca gatgtgactc aactttactt aggaggtatg
16441 agctattatt gtaaatcaca taaaccaccc attagttttc cattgtgtgc taatggacaa
16501 gtttttggtt tatataaaaa tacatgtgtt ggtagcgata atgttactga ctttaatgca
16561 attgcaacat gtgactggac aaatgctggt gattacattt tagctaacac ctgtactgaa
16621 agactcaagc tttttgcagc agaaacgctc aaagctactg aggagacatt taaactgtct
16681 tatggtattg ctactgtacg tgaagtgctg tctgacagag aattacatct ttcatgggaa
16741 gttggtaaac ctagaccacc acttaaccga aattatgtct ttactggtta tcgtgtaact
16801 aaaaacagta aagtacaaat aggagagtac acctttgaaa aaggtgacta tggtgatgct
16861 gttgtttacc gaggtacaac aacttacaaa ttaaatgttg gtgattattt tgtgctgaca
16921 tcacatacag taatgccatt aagtgcacct acactagtgc cacaagagca ctatgttaga
16981 attactggct tatacccaac actcaatatc tcagatgagt tttctagcaa tgttgcaaat
17041 tatcaaaagg ttggtatgca aaagtattct acactccagg gaccacctgg tactggtaag
17101 agtcattttg ctattggcct agctctctac tacccttctg ctcgcatagt gtatacagct
17161 tgctctcatg ccgctgttga tgcactatgt gagaaggcat taaaatattt gcctatagat
17221 aaatgtagta gaattatacc tgcacgtgct cgtgtagagt gttttgataa attcaaagtg
17281 aattcaacat tagaacagta tgtcttttgt actgtaaatg cattgcctga gacgacagca
17341 gatatagttg tctttgatga aatttcaatg gccacaaatt atgatttgag tgttgtcaat
17401 gccagattac gtgctaagca ctatgtgtac attggcgacc ctgctcaatt acctgcacca
17461 cgcacattgc taactaaggg cacactagaa ccagaatatt tcaattcagt gtgtagactt
17521 atgaaaacta taggtccaga catgttcctc ggaacttgtc ggcgttgtcc tgctgaaatt
17581 gttgacactg tgagtgcttt ggtttatgat aataagctta aagcacataa agacaaatca
17641 gctcaatgct ttaaaatgtt ttataagggt gttatcacgc atgatgtttc atctgcaatt
17701 aacaggccac aaataggcgt ggtaagagaa ttccttacac gtaaccctgc ttggagaaaa
17761 gctgtcttta tttcacctta taattcacag aatgctgtag cctcaaagat tttgggacta
17821 ccaactcaaa ctgttgattc atcacagggc tcagaatatg actatgtcat attcactcaa
17881 accactgaaa cagctcactc ttgtaatgta aacagattta atgttgctat taccagagca
17941 aaagtaggca tactttgcat aatgtctgat agagaccttt atgacaagtt gcaatttaca
18001 agtcttgaaa ttccacgtag gaatgtggca actttacaag ctgaaaatgt aacaggactt
18061 tttaaagatt gtagtaaggt aatcactggg ttacatccta cacaggcacc tacacacctc
18121 agtgttgaca ctaaattcaa aactgaaggt ttatgtgttg acatacctgg catacctaag
18181 gacatgacct atagaagact catctctatg atgggtttta aaatgaatta tcaagttaat
18241 ggttacccta acatgtttat cacccgcgaa gaagctataa gacatgtacg tgcatggatt
18301 ggcttcgatg tcgaggggtg tcatgctact agagaagctg ttggtaccaa tttaccttta
18361 cagctaggtt tttctacagg tgttaaccta gttgctgtac ctacaggtta tgttgataca
18421 cctaataata cagatttttc cagagttagt gctaaaccac cgcctggaga tcaatttaaa
18481 cacctcatac cacttatgta caaaggactt ccttggaatg tagtgcgtat aaagattgta
18541 caaatgttaa gtgacacact taaaaatctc tctgacagag tcgtatttgt cttatgggca
18601 catggctttg agttgacatc tatgaagtat tttgtgaaaa taggacctga gcgcacctgt
18661 tgtctatgtg atagacgtgc cacatgcttt tccactgctt cagacactta tgcctgttgg
18721 catcattcta ttggatttga ttacgtctat aatccgttta tgattgatgt tcaacaatgg
18781 ggttttacag gtaacctaca aagcaaccat gatctgtatt gtcaagtcca tggtaatgca
18841 catgtagcta gttgtgatgc aatcatgact aggtgtctag ctgtccacga gtgctttgtt
18901 aagcgtgttg actggactat tgaatatcct ataattggtg atgaactgaa gattaatgcg
18961 gcttgtagaa aggttcaaca catggttgtt aaagctgcat tattagcaga caaattccca
19021 gttcttcacg acattggtaa ccctaaagct attaagtgtg tacctcaagc tgatgtagaa
19081 tggaagttct atgatgcaca gccttgtagt gacaaagctt ataaaataga agaattattc
19141 tattcttatg ccacacattc tgacaaattc acagatggtg tatgcctatt ttggaattgc
19201 aatgtcgata gatatcctgc taattccatt gtttgtagat ttgacactag agtgctatct
19261 aaccttaact tgcctggttg tgatggtggc agtttgtatg taaataaaca tgcattccac
19321 acaccagctt ttgataaaag tgcttttgtt aatttaaaac aattaccatt tttctattac
19381 tctgacagtc catgtgagtc tcatggaaaa caagtagtgt cagatataga ttatgtacca
19441 ctaaagtctg ctacgtgtat aacacgttgc aatttaggtg gtgctgtctg tagacatcat
19501 gctaatgagt acagattgta tctcgatgct tataacatga tgatctcagc tggctttagc
19561 ttgtgggttt acaaacaatt tgatacttat aacctctgga acacttttac aagacttcag
19621 agtttagaaa atgtggcttt taatgttgta aataagggac actttgatgg acaacagggt
19681 gaagtaccag tttctatcat taataacact gtttacacaa aagttgatgg tgttgatgta
19741 gaattgtttg aaaataaaac aacattacct gttaatgtag catttgagct ttgggctaag
19801 cgcaacatta aaccagtacc agaggtgaaa atactcaata atttgggtgt ggacattgct
19861 gctaatactg tgatctggga ctacaaaaga gatgctccag cacatatatc tactattggt
19921 gtttgttcta tgactgacat agccaagaaa ccaactgaaa cgatttgtgc accactcact
19981 gtcttttttg atggtagagt tgatggtcaa gtagacttat ttagaaatgc ccgtaatggt
20041 gttcttatta cagaaggtag tgttaaaggt ttacaaccat ctgtaggtcc caaacaagct
20101 agtcttaatg gagtcacatt aattggagaa gccgtaaaaa cacagttcaa ttattataag
20161 aaagttgatg gtgttgtcca acaattacct gaaacttact ttactcagag tagaaattta
20221 caagaattta aacccaggag tcaaatggaa attgatttct tagaattagc tatggatgaa
20281 ttcattgaac ggtataaatt agaaggctat gccttcgaac atatcgttta tggagatttt
20341 agtcatagtc agttaggtgg tttacatcta ctgattggac tagctaaacg ttttaaggaa
20401 tcaccttttg aattagaaga ttttattcct atggacagta cagttaaaaa ctatttcata
20461 acagatgcgc aaacaggttc atctaagtgt gtgtgttctg ttattgattt attacttgat
20521 gattttgttg aaataataaa atcccaagat ttatctgtag tttctaaggt tgtcaaagtg
20581 actattgact atacagaaat ttcatttatg ctttggtgta aagatggcca tgtagaaaca
20641 ttttacccaa aattacaatc tagtcaagcg tggcaaccgg gtgttgctat gcctaatctt
20701 tacaaaatgc aaagaatgct attagaaaag tgtgaccttc aaaattatgg tgatagtgca
20761 acattaccta aaggcataat gatgaatgtc gcaaaatata ctcaactgtg tcaatattta
20821 aacacattaa cattagctgt accctataat atgagagtta tacattttgg tgctggttct
20881 gataaaggag ttgcaccagg tacagctgtt ttaagacagt ggttgcctac gggtacgctg
20941 cttgtcgatt cagatcttaa tgactttgtc tctgatgcag attcaacttt gattggtgat
21001 tgtgcaactg tacatacagc taataaatgg gatctcatta ttagtgatat gtacgaccct
21061 aagactaaaa atgttacaaa agaaaatgac tctaaagagg gttttttcac ttacatttgt
21121 gggtttatac aacaaaagct agctcttgga ggttccgtgg ctataaagat aacagaacat
21181 tcttggaatg ctgatcttta taagctcatg ggacacttcg catggtggac agcctttgtt
21241 actaatgtga atgcgtcatc atctgaagca tttttaattg gatgtaatta tcttggcaaa
21301 ccacgcgaac aaatagatgg ttatgtcatg catgcaaatt acatattttg gaggaataca
21361 aatccaattc agttgtcttc ctattcttta tttgacatga gtaaatttcc ccttaaatta
21421 aggggtactg ctgttatgtc tttaaaagaa ggtcaaatca atgatatgat tttatctctt
21481 cttagtaaag gtagacttat aattagagaa aacaacagag ttgttatttc tagtgatgtt
21541 cttgttaaca actaaacgaa caatgtttgt ttttcttgtt ttattgccac tagtctctag
21601 tcagtgtgtt aatcttacaa ccagaactca attaccccct gcatacacta attctttcac
21661 acgtggtgtt tattaccctg acaaagtttt cagatcctca gttttacatt caactcagga
21721 cttgttctta cctttctttt ccaatgttac ttggttccat gctatacatg tctctgggac
21781 caatggtact aagaggtttg ataaccctgt cctaccattt aatgatggtg tttattttgc
21841 ttccactgag aagtctaaca taataagagg ctggattttt ggtactactt tagattcgaa
21901 gacccagtcc ctacttattg ttaataacgc tactaatgtt gttattaaag tctgtgaatt
21961 tcaattttgt aatgatccat ttttgggtgt ttattaccac aaaaacaaca aaagttggat
22021 ggaaagtgag ttcagagttt attctagtgc gaataattgc acttttgaat atgtctctca
22081 gccttttctt atggaccttg aaggaaaaca gggtaatttc aaaaatctta gggaatttgt
22141 gtttaagaat attgatggtt attttaaaat atattctaag cacacgccta ttaatttagt
22201 gcgtgatctc cctcagggtt tttcggcttt agaaccattg gtagatttgc caataggtat
22261 taacatcact aggtttcaaa ctttacttgc tttacataga agttatttga ctcctggtga
22321 ttcttcttca ggttggacag ctggtgctgc agcttattat gtgggttatc ttcaacctag
22381 gacttttcta ttaaaatata atgaaaatgg aaccattaca gatgctgtag actgtgcact
22441 tgaccctctc tcagaaacaa agtgtacgtt gaaatccttc actgtagaaa aaggaatcta
22501 tcaaacttct aactttagag tccaaccaac agaatctatt gttagatttc ctaatattac
22561 aaacttgtgc ccttttggtg aagtttttaa cgccaccaga tttgcatctg tttatgcttg
22621 gaacaggaag agaatcagca actgtgttgc tgattattct gtcctatata attccgcatc
22681 attttccact tttaagtgtt atggagtgtc tcctactaaa ttaaatgatc tctgctttac
22741 taatgtctat gcagattcat ttgtaattag aggtgatgaa gtcagacaaa tcgctccagg
22801 gcaaactgga aagattgctg attataatta taaattacca gatgatttta caggctgcgt
22861 tatagcttgg aattctaaca atcttgattc taaggttggt ggtaattata attacctgta
22921 tagattgttt aggaagtcta atctcaaacc ttttgagaga gatatttcaa ctgaaatcta
22981 tcaggccggt agcacacctt gtaatggtgt tgaaggtttt aattgttact ttcctttaca
23041 atcatatggt ttccaaccca ctaatggtgt tggttaccaa ccatacagag tagtagtact
23101 ttcttttgaa cttctacatg caccagcaac tgtttgtgga cctaaaaagt ctactaattt
23161 ggttaaaaac aaatgtgtca atttcaactt caatggttta acaggcacag gtgttcttac
23221 tgagtctaac aaaaagtttc tgcctttcca acaatttggc agagacattg ctgacactac
23281 tgatgctgtc cgtgatccac agacacttga gattcttgac attacaccat gttcttttgg
23341 tggtgtcagt gttataacac caggaacaaa tacttctaac caggttgctg ttctttatca
23401 ggatgttaac tgcacagaag tccctgttgc tattcatgca gatcaactta ctcctacttg
23461 gcgtgtttat tctacaggtt ctaatgtttt tcaaacacgt gcaggctgtt taataggggc
23521 tgaacatgtc aacaactcat atgagtgtga catacccatt ggtgcaggta tatgcgctag
23581 ttatcagact cagactaatt ctcctcggcg ggcacgtagt gtagctagtc aatccatcat
23641 tgcctacact atgtcacttg gtgcagaaaa ttcagttgct tactctaata actctattgc
23701 catacccaca aattttacta ttagtgttac cacagaaatt ctaccagtgt ctatgaccaa
23761 gacatcagta gattgtacaa tgtacatttg tggtgattca actgaatgca gcaatctttt
23821 gttgcaatat ggcagttttt gtacacaatt aaaccgtgct ttaactggaa tagctgttga
23881 acaagacaaa aacacccaag aagtttttgc acaagtcaaa caaatttaca aaacaccacc
23941 aattaaagat tttggtggtt ttaatttttc acaaatatta ccagatccat caaaaccaag
24001 caagaggtca tttattgaag atctactttt caacaaagtg acacttgcag atgctggctt
24061 catcaaacaa tatggtgatt gccttggtga tattgctgct agagacctca tttgtgcaca
24121 aaagtttaac ggccttactg ttttgccacc tttgctcaca gatgaaatga ttgctcaata
24181 cacttctgca ctgttagcgg gtacaatcac ttctggttgg acctttggtg caggtgctgc
24241 attacaaata ccatttgcta tgcaaatggc ttataggttt aatggtattg gagttacaca
24301 gaatgttctc tatgagaacc aaaaattgat tgccaaccaa tttaatagtg ctattggcaa
24361 aattcaagac tcactttctt ccacagcaag tgcacttgga aaacttcaag atgtggtcaa
24421 ccaaaatgca caagctttaa acacgcttgt taaacaactt agctccaatt ttggtgcaat
24481 ttcaagtgtt ttaaatgata tcctttcacg tcttgacaaa gttgaggctg aagtgcaaat
24541 tgataggttg atcacaggca gacttcaaag tttgcagaca tatgtgactc aacaattaat
24601 tagagctgca gaaatcagag cttctgctaa tcttgctgct actaaaatgt cagagtgtgt
24661 acttggacaa tcaaaaagag ttgatttttg tggaaagggc tatcatctta tgtccttccc
24721 tcagtcagca cctcatggtg tagtcttctt gcatgtgact tatgtccctg cacaagaaaa
24781 gaacttcaca actgctcctg ccatttgtca tgatggaaaa gcacactttc ctcgtgaagg
24841 tgtctttgtt tcaaatggca cacactggtt tgtaacacaa aggaattttt atgaaccaca
24901 aatcattact acagacaaca catttgtgtc tggtaactgt gatgttgtaa taggaattgt
24961 caacaacaca gtttatgatc ctttgcaacc tgaattagac tcattcaagg aggagttaga
25021 taaatatttt aagaatcata catcaccaga tgttgattta ggtgacatct ctggcattaa
25081 tgcttcagtt gtaaacattc aaaaagaaat tgaccgcctc aatgaggttg ccaagaattt
25141 aaatgaatct ctcatcgatc tccaagaact tggaaagtat gagcagtata taaaatggcc
25201 atggtacatt tggctaggtt ttatagctgg cttgattgcc atagtaatgg tgacaattat
25261 gctttgctgt atgaccagtt gctgtagttg tctcaagggc tgttgttctt gtggatcctg
25321 ctgcaaattt gatgaagacg actctgagcc agtgctcaaa ggagtcaaat tacattacac
25381 ataaacgaac ttatggattt gtttatgaga atcttcacaa ttggaactgt aactttgaag
25441 caaggtgaaa tcaaggatgc tactccttca gattttgttc gcgctactgc aacgataccg
25501 atacaagcct cactcccttt cggatggctt attgttggcg ttgcacttct tgctgttttt
25561 cagagcgctt ccaaaatcat aaccctcaaa aagagatggc aactagcact ctccaagggt
25621 gttcactttg tttgcaactt gctgttgttg tttgtaacag tttactcaca ccttttgctc
25681 gttgctgctg gccttgaagc cccttttctc tatctttatg ctttagtcta cttcttgcag
25741 agtataaact ttgtaagaat aataatgagg ctttggcttt gctggaaatg ccgttccaaa
25801 aacccattac tttatgatgc caactatttt ctttgctggc atactaattg ttacgactat
25861 tgtatacctt acaatagtgt aacttcttca attgtcatta cttcaggtga tggcacaaca
25921 agtcctattt ctgaacatga ctaccagatt ggtggttata ctgaaaaatg ggaatctgga
25981 gtaaaagact gtgttgtatt acacagttac ttcacttcag actattacca gctgtactca
26041 actcaattga gtacagacac tggtgttgaa catgttacct tcttcatcta caataaaatt
26101 gttgatgagc ctgaagaaca tgtccaaatt cacacaatcg acggttcatc cggagttgtt
26161 aatccagtaa tggaaccaat ttatgatgaa ccgacgacga ctactagcgt gcctttgtaa
26221 gcacaagctg atgagtacga acttatgtac tcattcgttt cggaagagac aggtacgtta
26281 atagttaata gcgtacttct ttttcttgct ttcgtggtat tcttgctagt tacactagcc
26341 atccttactg cgcttcgatt gtgtgcgtac tgctgcaata ttgttaacgt gagtcttgta
26401 aaaccttctt tttacgttta ctctcgtgtt aaaaatctga attcttctag agttcctgat
26461 cttctggtct aaacgaacta aatattatat tagtttttct gtttggaact ttaattttag
26521 ccatggcaga ttccaacggt actattaccg ttgaagagct taaaaagctc cttgaacaat
26581 ggaacctagt aataggtttc ctattcctta catggatttg tcttctacaa tttgcctatg
26641 ccaacaggaa taggtttttg tatataatta agttaatttt cctctggctg ttatggccag
26701 taactttagc ttgttttgtg cttgctgctg tttacagaat aaattggatc accggtggaa
26761 ttgctatcgc aatggcttgt cttgtaggct tgatgtggct cagctacttc attgcttctt
26821 tcagactgtt tgcgcgtacg cgttccatgt ggtcattcaa tccagaaact aacattcttc
26881 tcaacgtgcc actccatggc actattctga ccagaccgct tctagaaagt gaactcgtaa
26941 tcggagctgt gatccttcgt ggacatcttc gtattgctgg acaccatcta ggacgctgtg
27001 acatcaagga cctgcctaaa gaaatcactg ttgctacatc acgaacgctt tcttattaca
27061 aattgggagc ttcgcagcgt gtagcaggtg actcaggttt tgctgcatac agtcgctaca
27121 ggattggcaa ctataaatta aacacagacc attccagtag cagtgacaat attgctttgc
27181 ttgtacagta agtgacaaca gatgtttcat ctcgttgact ttcaggttac tatagcagag
27241 atattactaa ttattatgag gacttttaaa gtttccattt ggaatcttga ttacatcata
27301 aacctcataa ttaaaaattt atctaagtca ctaactgaga ataaatattc tcaattagat
27361 gaagagcaac caatggagat tgattaaacg aacatgaaaa ttattctttt cttggcactg
27421 ataacactcg ctacttgtga gctttatcac taccaagagt gtgttagagg tacaacagta
27481 cttttaaaag aaccttgctc ttctggaaca tacgagggca attcaccatt tcatcctcta
27541 gctgataaca aatttgcact gacttgcttt agcactcaat ttgcttttgc ttgtcctgac
27601 ggcgtaaaac acgtctatca gttacgtgcc agatcagttt cacctaaact gttcatcaga
27661 caagaggaag ttcaagaact ttactctcca atttttctta ttgttgcggc aatagtgttt
27721 ataacacttt gcttcacact caaaagaaag acagaatgat tgaactttca ttaattgact
27781 tctatttgtg ctttttagcc tttctgctat tccttgtttt aattatgctt attatctttt
27841 ggttctcact tgaactgcaa gatcataatg aaacttgtca cgcctaaacg aacatgaaat
27901 ttcttgtttt cttaggaatc atcacaactg tagctgcatt tcaccaagaa tgtagtttac
27961 agtcatgtac tcaacatcaa ccatatgtag ttgatgaccc gtgtcctatt cacttctatt
28021 ctaaatggta tattagagta ggagctagaa aatcagcacc tttaattgaa ttgtgcgtgg
28081 atgaggctgg ttctaaatca cccattcagt acatcgatat cggtaattat acagtttcct
28141 gttcaccttt tacaattaat tgccaggaac ctaaattggg tagtcttgta gtgcgttgtt
28201 cgttctatga agacttttta gagtatcatg acgttcgtgt tgttttagat ttcatctaaa
28261 cgaacaaact aaaatgtctg ataatggacc ccaaaatcag cgaaatgcac cccgcattac
28321 gtttggtgga ccctcagatt caactggcag taaccagaat ggagaacgca gtggggcgcg
28381 atcaaaacaa cgtcggcccc aaggtttacc caataatact gcgtcttggt tcaccgctct
28441 cactcaacat ggcaaggaag accttaaatt ccctcgagga caaggcgttc caattaacac
28501 caatagcagt ccagatgacc aaattggcta ctaccgaaga gctaccagac gaattcgtgg
28561 tggtgacggt aaaatgaaag atctcagtcc aagatggtat ttctactacc taggaactgg
28621 gccagaagct ggacttccct atggtgctaa caaagacggc atcatatggg ttgcaactga
28681 gggagccttg aatacaccaa aagatcacat tggcacccgc aatcctgcta acaatgctgc
28741 aatcgtgcta caacttcctc aaggaacaac attgccaaaa ggcttctacg cagaagggag
28801 cagaggcggc agtcaagcct cttctcgttc ctcatcacgt agtcgcaaca gttcaagaaa
28861 ttcaactcca ggcagcagta ggggaacttc tcctgctaga atggctggca atggcggtga
28921 tgctgctctt gctttgctgc tgcttgacag attgaaccag cttgagagca aaatgtctgg
28981 taaaggccaa caacaacaag gccaaactgt cactaagaaa tctgctgctg aggcttctaa
29041 gaagcctcgg caaaaacgta ctgccactaa agcatacaat gtaacacaag ctttcggcag
29101 acgtggtcca gaacaaaccc aaggaaattt tggggaccag gaactaatca gacaaggaac
29161 tgattacaaa cattggccgc aaattgcaca atttgccccc agcgcttcag cgttcttcgg
29221 aatgtcgcgc attggcatgg aagtcacacc ttcgggaacg tggttgacct acacaggtgc
29281 catcaaattg gatgacaaag atccaaattt caaagatcaa gtcattttgc tgaataagca
29341 tattgacgca tacaaaacat tcccaccaac agagcctaaa aaggacaaaa agaagaaggc
29401 tgatgaaact caagccttac cgcagagaca gaagaaacag caaactgtga ctcttcttcc
29461 tgctgcagat ttggatgatt tctccaaaca attgcaacaa tccatgagca gtgctgactc
29521 aactcaggcc taaactcatg cagaccacac aaggcagatg ggctatataa acgttttcgc
29581 ttttccgttt acgatatata gtctactctt gtgcagaatg aattctcgta actacatagc
29641 acaagtagat gtagttaact ttaatctcac atagcaatct ttaatcagtg tgtaacatta
29701 gggaggactt gaaagagcca ccacattttc accgaggcca cgcggagtac gatcgagtgt
29761 acagtgaaca atgctaggga gagctgccta tatggaagag ccctaatgtg taaaattaat
29821 tttagtagtg ctatccccat gtgattttaa tagcttctta ggagaatgac aaaaaaaaaa
29881 aa
Delta: Genbank MZ888544.1
Nucleic Acid Sequence
(SEQ ID NOs: 4-7)
        1 aaccaacttt cgatctcttg tagatctgtt ctctaaacga actttaaaat ctgtgtggct
      61 gtcactcggc tgcatgctta gtgcactcac gcagtataat taataactaa ttactgtcgt
    121 tgacaggaca cgagtaactc gtctatcttc tgcaggctgc ttacggtttc gtccgttttg
    181 cagccgatca tcagcacatc taggttttgt ccgggtgtga ccgaaaggta agatggagag
    241 ccttgtccct ggtttcaacg agaaaacaca cgtccaactc agtttgcctg ttttacaggt
    301 tcgcgacgtg ctcgtacgtg gctttggaga ctccgtggag gaggtcttat cagaggcacg
    361 tcaacatctt aaagatggca cttgtggctt agtagaagtt gaaaaaggcg ttttgcctca
    421 acttgaacag ccctatgtgt tcatcaaacg ttcggatgct cgaactgcac ctcatggtca
    481 tgttatggtt gagctggtag cagaactcga aggcattcag tacggtcgta gtggtgagac
    541 acttggtgtc cttgtccctc atgtgggcga aataccagtg gcttaccgca aggttcttct
    601 tcgtaagaac ggtaataaag gagctggtgg ccatagttac ggcgccgatc taaagtcatt
    661 tgacttaggc gacgggcttg gcactgatcc ttatgaagat tttcaagaaa actggaacac
    721 taaacatagc agtggtgtta cccgtgaact catgcgtgag cttaacggag gggcatacac
    781 tcgctatgtc gataacaact tctgtggccc tgatggctac cctcttgagt gcattaaaga
    841 ccttctagca cgtgctggta aagcttcatg cactttgtcc gaacaactgg actttattga
    901 cactaagagg ggtgtatact gctgccgtga acatgagcat gaaattgctt ggtacacgga
    961 acgttctgaa aagagctatg aattgcagac accttttgaa attaaattgg caaagaaatt
  1021 tgacaccttc aatggggaat gtccaaattt tgtatttccc ttaaattcca taatcaagac
  1081 tattcaacca agggttgaaa agaaaaagct tgatggcttt atgggtagaa ttcgatctgt
  1141 ctatccagtt gcgtcaccaa atgaatgcaa ccaaatgtgc ctttcaactc tcatgaagtg
  1201 tgatcattgt ggtgaaactt catggcagac gggcgatttt gttaaagcca cttgcgaatt
  1261 ttgtggcact gagaatttga ctaaagaagg tgccactact tgtggttact taccccaaaa
  1321 tgctgttgtt aaaatttatt gtccagcatg tcacaattca gaagtaggac ctgagcatag
  1381 tcttgccgaa taccataatg aatctggctt gaaaaccatt cttcgtaagg gtggtcgcac
  1441 tattgccttt ggaggctgtg tgttctctta tgttggttgc cataacaagt gtgcctattg
  1501 ggttccacgt gctagcgcta acataggttg taaccataca ggtgttgttg gagaaggttc
  1561 cgaaggtctt aatgacaacc ttcttgaaat actccaaaaa gagaaagtca acatcaatat
  1621 tgttggtgac tttaaactta atgaagagat cgccattatt ttggcatctt tttctgcttc
  1681 cacaagtgct tttgtggaaa ctgtgaaagg tttggattat aaagcattca aacaaattgt
  1741 tgaatcctgt ggtaatttta aagttacaaa aggaaaagct aaaaaaggtg cttggaatat
  1801 tggtgaacag aaatcaatac tgagtcctct ttatgcattt gcatcagagg ctgctcgtgt
  1861 tgtacgatca attttctccc gcactcttga aactgctcaa aattctgtgc gtgttttaca
  1921 gaaggccgct ataacaatac tagatggaat ttcacagtat tcactgagac tcattgatgc
  1981 tatgatgttc acatctgatt tggctactaa caatctagtt gtaatggcct acattacagg
  2041 tggtgttgtt cagttgactt cgcagtggct aactaacatc tttggcactg tttatgaaaa
  2101 actcaaaccc gtccttgatt ggcttgaaga gaagtttaag gaaggtgtag agtttcttag
  2161 agacggttgg gaaattgtta aatttatctc aacctgtgct tgtgaaattg tcggtggaca
  2221 aattgtcacc tgtgcaaagg aaattaagga gagtgttcag acattcttta agcttgtaaa
  2281 taaatttttg gctttgtgtg ctgactctat cattattggt ggagctaaac ttaaagcctt
  2341 gaatttaggt gaaacatttg tcacgcactc aaagggattg tacagaaagt gtgttaaatc
  2401 cagagaagaa actggcctac tcatgcctct aaaagcccca aaagaaatta tcttcttaga
  2461 gggagaaaca cttcccacag aagtgttaac agaggaagtt gtcttgaaaa ctggtgattt
  2521 acaaccatta gaacaaccta ctagtgaagc tgttgaagct ccattggttg gtacaccagt
  2581 ttgtattaac gggcttatgt tgctcgaaat caaagacaca gaaaagtact gtgcccttgc
  2641 acctaatatg atggtaacaa acaatacctt cacactcaaa ggcggtgcac caacaaaggt
  2701 tacttttggt gatgacactg tgatagaagt gcaaggttac aagagtgtga atatcacttt
  2761 tgaacttgat gaaaggattg ataaagtact taatgagaag tgctctgcct atacagttga
  2821 actcggtaca gaagtaaatg agttcgcctg tgttgtggca gatgctgtca taaaaacttt
  2881 gcaaccagta tctgaattac ttacaccact gggcattgat ttagatgagt ggagtatggc
  2941 tacatactac ttatttgatg agtctggtga gtttaaattg gcttcacata tgtattgttc
  3001 tttttaccct ccagatgagg atgaagaaga aggtgattgt gaagaagaag agtttgagcc
  3061 atcaactcaa tatgagtatg gtactgaaga tgattaccaa ggtaaacctt tggaatttgg
  3121 tgccacttct gctgctcttc aacctgaaga agagcaagaa gaagattggt tagatgatga
  3181 tagtcaacaa actgttggtc aacaagacgg cagtgaggac aatcagacaa ctactattca
  3241 aacaattgtt gaggttcaac ctcaattaga gatggaactt acaccagttg ttcagactat
  3301 tgaagtgaat agttttagtg gttatttaaa acttactgac aatgtataca ttaaaaatgc
  3361 agacattgtg gaagaagtta aaaaggtaaa accaacagtg gttgttaatg cagccaatgt
  3421 ttaccttaaa catggaggag gtgttgcagg agccttaaat aaggctacta acaatgccat
  3481 gcaagttgaa tctgatgatt acatagctac taatggacca cttaaagtgg gtggtagttg
  3541 tgttttaagc ggacacaatc ttgctaaaca ctgtcttcat gttgtcggcc caaatgttaa
  3601 caaaggtgaa gacattcaac ttcttaagag tgcttatgaa aattttaatc agcacgaagt
  3661 tctacttgca ccattattat cagctggtat ttttggtgct gaccctatac attctttaag
  3721 agtttgtgta gatactgttc gcacaaatgt ctacttagct gtctttgata aaaatctcta
  3781 tgacaaactt gtttcaagct ttttggaaat gaagagtgaa aagcaagttg aacaaaagat
  3841 cgctgagatt cctaaagagg aagttaagcc atttataact gaaagtaaac cttcagttga
  3901 acagagaaaa caagatgata agaaaatcaa agcttgtgtt gaagaagtta caacaactct
  3961 ggaagaaact aagttcctca cagaaaactt gttactttat attgacatta atggcaatct
  4021 tcatccagat tctgccactc ttgttagtga cattgacatc actttcttaa agaaagatgc
  4081 tccatatata gtgggtgatg ttgttcaaga gggtgtttta actgctgtgg ttatacctac
  4141 taaaaagtct ggtggcacta ctgaaatgct agcgaaagct ttgagaaaag tgccaacaga
  4201 caattatata accacttacc cgggtcaggg tttaaatggt tacactgtag aggaggcaaa
  4261 gacagtgctt aaaaagtgta aaagtgcctt ttacattcta ccatctatta tctctaatga
  4321 gaagcaagaa attcttggaa ctgtttcttg gaatttgcga gaaatgcttg cacatgcaga
  4381 agaaacacgc aaattaatgc ctgtctgtgt ggaaactaaa gccatagttt caactataca
  4441 gcgtaaatat aagggtatta aaatacaaga gggtgtggtt gattatggtg ctagatttta
  4501 cttttacacc agtaaaacaa ctgtagcgtc acttatcaac acacttaacg atctaaatga
  4561 aactcttgtt acaatgccac ttggctatgt aacacatggc ttaaatttgg aagaagctgc
  4621 tcggtatatg agatctctca aagtgccagc tacagtttct gtttcttcac ctgatgctgt
  4681 tacagcgtat aatggttatc ttacttcttc ttctaaaaca cctgaagaac attttattga
  4741 aaccatctca cttgctggtt cctataaaga ttggtcctat tctggacaat ctacacaact
  4801 aggtatagaa tttcttaaga gaggtgataa aagtgtatat tacactagta atcctaccac
  4861 attccaccta gatggtgaag ttatcacctt tgacaatctt aagacacttc tttctttgag
  4921 agaagtgagg actattaagg tgtttacaac agtagacaac attaacctcc acacgcaagt
  4981 tgtggacatg tcaatgacat atggacaaca gtttggtcca acttatttgg atggagctga
  5041 tgttactaaa ataaaacctc ataattcaca tgaaggtaaa acattttatg ttttacctaa
  5101 tgatgacact ctacgtgttg aggcttttga gtactaccac acaactgatc ctagttttct
  5161 gggtaggtac atgtcagcat taaatcacac taaaaagtgg aaatacccac aagttaatgg
  5221 tttaacttct attaaatggg cagataacaa ctgttatctt gccactgcat tgttaacact
  5281 ccaacaaata gagttgaagt ttaatccacc tgctctacaa gatgcttatt acagagcaag
  5341 ggctggtgaa gctgctaact tttgtgcact tatcttagcc tactgtaata agacagtagg
  5401 tgagttaggt gatgttagag aaacaatgag ttacttgttt caacatgcca atttagattc
  5461 ttgcaaaaga gtcttgaacg tggtgtgtaa aacttgtgga caacagcaga caacccttaa
  5521 gggtgtagaa gctgttatgt acatgggcac actttcttat gaacaattta agaaaggtgt
  5581 tcagatacct tgtacgtgtg gtaaacaagc tacaaaatat ctagtacaac aggagtcacc
  5641 ttttgttatg atgtcagcac cacctgctca gtatgaactt aagcatggta catttacttg
  5701 tgctagtgag tacactggta attaccagtg tggtcactat aaacatataa cttctaaaga
  5761 aactttgtat tgcatagacg gtgctttact tacaaagtcc tcagaataca aaggtcctat
  5821 tacggatgtt ttctacaaag aaaacagtta cacaacaacc ataaaaccag ttacttataa
  5881 attggatggt gttgtttgta cagaaattga ccctaagttg gacaattatt ataagaaaga
  5941 caattcttat ttcacagagc aaccaattga tcttgtacca aaccaaccat atccaaacgc
  6001 aagcttcgat aattttaagt ttgtatgtga taatatcaaa tttgctgatg atttaaacca
  6061 gttaactggt tataagaaac ctgcttcaag agagcttaaa gttacatttt tccctgactt
  6121 aaatggtgat gtggtggcta ttgattataa acactacaca ccctctttta agaaaggagc
  6181 taaattgtta cataaaccta ttgtttggca tgttaacaat gcaactaata aagccacgta
  6241 taaaccaaat acctggtgta tacgttgtct ttggagcaca aaaccagttg aaacatcaaa
  6301 ttcgtttgat gtactgaagt cagaggacgc gcagggaatg gataatcttg cctgcgaaga
  6361 tctaaaacta gtctctgaag aagtagtgga aaatcctacc atacagaaag acgttcttga
  6421 gtgtaatgtg aaaactaccg aagttgtagg agacattata cttaaaccag caaataatag
  6481 tttaaaaatt acagaagagg ttggccacac agatctaatg gctgcttatg tagacaattc
  6541 tagtcttact attaagaaac ctaatgaatt atctagagta ttaggtttga aaacccttgc
  6601 tactcatggt ttagctgctg ttaatagtgt cccttgggat actatagcta attatgctaa
  6661 gccttttctt aacaaagttg ttagtacaac tactaacata gttacacggt gtttaaaccg
  6721 tgtttgtact aattatatgc cttatttctt tactttattg ctacaattgt gtacttttac
  6781 tagaagtaca aattctagaa ttaaagcatc tatgccgact actatagcaa agaatactgt
  6841 taagagtgtc ggtaaatttt gtctagaggc ttcatttaat tatttgaagt cacctaattt
  6901 ttctaaactg ataaatatta taatttggtt tttactatta agtgtttgcc taggttcttt
  6961 aatctactca accgctgctt taggtgtttt aatgtctaat ttaggcatgc cttcttactg
  7021 tactggttac agagaaggct atttgaactc tactaatgtc actattgcaa cctactgtac
  7081 tggttctata tcttgtagtg tttgtcttag tggtttagat tctttagaca cctatccttc
  7141 tttagaaact atacaaatta ccatttcatc ttttaaatgg gatttaactg cttttggctt
  7201 agttgcagag tggtttttgg catatattct tttcactagg tttttctatg tacttggatt
  7261 ggctgcaatc atgcaattgt ttttcagcta ttttgcagta cattttatta gtaattcttg
  7321 gcttatgtgg ttaataatta atcttgtaca aatggccccg atttcagcta tggttagaat
  7381 gtacatcttc tttgcatcat tttattatgt atggaaaagt tatgtgcatg ttgtagacgg
  7441 ttgtaattca tcaacttgta tgatgtgtta caaacgtaat agagcaacaa gagtcgaatg
  7501 tacaactatt gttaatggtg ttagaaggtc cttttatgtc tatgctaatg gaggtaaagg
  7561 cttttgcaaa ctacacaatt ggaattgtgt taattgtgat acattctgtg ctggtagtac
  7621 atttattagt gatgaagttg cgagagactt gtcactacag tttaaaagac caataaatcc
  7681 tactgaccag tcttcttaca tcgttgatag tgttacagtg aagaatggtt ccatccatct
  7741 ttactttgat aaagctggtc aaaagactta tgaaagacat tctctctctc attttgttaa
  7801 cttagacaac ctgagagcta ataacactaa aggttcattg cctattaatg ttatagtttt
  7861 tgatggtaaa tcaaaatgtg aagaatcatc tgcaaaatca gcgtctgttt actacagtca
  7921 gcttatgtgt caacctatac tgttactaga tcaggcatta gtgtctgatg ttggtgatag
  7981 tgcggaagtt gcagttaaaa tgtttgatgc ttacgttaat acgttttcat caacttttaa
  8041 cgtaccaatg gaaaaactca aaacactagt tgcaactgca gaagctgaac ttgcaaagaa
  8101 tgtgtcctta gacaatgtct tatctacttt tatttcagca gctcggcaag ggtttgttga
  8161 ttcagatgta gaaactaaag atgttgttga atgtcttaaa ttgtcacatc aatctgacat
  8221 agaagttact ggcgatagtt gtaataacta tatgctcacc tataacaaag ttgaaaacat
  8281 gacaccccgt gaccttggtg cttgtattga ctgtagtgcg cgtcatatta atgcgcaggt
  8341 agcaaaaagt cacaacattg ctttgatatg gaacgttaaa gatttcatgt cattgtctga
  8401 acaactacga aaacaaatac gtagtgctgc taaaaagaat aacttacctt ttaagttgac
  8461 atgtgcaact actagacaag ttgttaatgt tgtaacaaca aagatagcac ttaagggtgg
  8521 taaaattgtt aataattggt tgaagcagtt aattaaagtt acacttgtgt tcctttttgt
  8581 tgctgctatt ttctatttaa taacacctgt tcatgtcatg tctaaacata ctgacttttc
  8641 aagtgaaatc ataggataca aggctattga tggtggtgtc actcgtgaca tagcatctac
  8701 agatacttgt tttgctaaca aacatgctga ttttgacaca tggtttagcc agcgtggtgg
  8761 tagttatact aatgacaaag cttgcccatt gattgctgca gtcataacaa gagaagtggg
  8821 ttttgtcgtg cctggtttgc ctggcacgat attacgcaca actaatggtg actttttgca
  8881 tttcttacct agagttttta gtgcagttgg taacatctgt tacacaccat caaaacttat
  8941 agagtacact gattttgcaa catcagcttg tgttttggct gctgaatgta caatttttaa
  9001 agatgcttct ggtaagccat taccatattg ttatgatacc aatgtactag aaggttctgt
  9061 tgcttatgaa agtttacgcc ctgacacacg ttatgtgctc atggatggct ctattattca
  9121 atttcctaac acctaccttg aaggttctgt tagagtggta acaacttttg attctgagta
  9181 ctgtaggcac ggcacttgtg aaagatcaga agctggtgtt tgtgtatcta ctagtggtag
  9241 atgggtactt aacaatgatt attacagatc tttaccagga gttttctgtg gtgtagatgc
  9301 tgtaaattta cttactaata tgtttacacc actaattcaa cctattggtg ctttggacat
  9361 atcagcatct atagtagctg gtggtattgt agctatcgta gtaacatgcc ttgcctacta
  9421 ttttatgagg tttagaagag cttttggtga atacagtcat gtagttgcct ttaatacttt
  9481 actattcctt atgtcattca ctgtactctg tttaacacca gtttactcat tcttacctgg
  9541 tgtttattct gttatttact tgtacttgac attttatctt actaatgatg tttctttttt
  9601 agcacatatt cagtggatgg ttatgttcac acctttagta cctttctgga taacaattgc
  9661 ttatatcatt tgtatttcca caaagcattt ctattggttc tttagtaatt acctaaagag
  9721 acgtgtagtc tttaatggtg tttcctttag tacttttgaa gaagctgcgc tgtgcacctt
  9781 tttgttaaat aaagaaatgt atctaaagtt gcgtagtgat gtgctattac ctcttacgca
  9841 atataataga tacttagctc tttataataa gtacaagtat tttagtggag caatggatac
  9901 aactagctac agagaagctg cttgttgtca tctcgcaaag gctctcaatg acttcagtaa
  9961 ctcaggttct gatgttcttt accaaccacc acaaatctct atcacctcag ctgttttgca
10021 gagtggtttt agaaaaatgg cattcccatc tggtaaagtt gagggttgta tggtacaagt
10081 aacttgtggt acaactacac ttaacggtct ttggcttgat gacgtagttt actgtccaag
10141 acatgtgatc tgcacctctg aagacatgct taaccctaat tatgaagatt tactcattcg
10201 taagtctaat cataatttct tggtacaggc tggtaatgtt caactcaggg ttattggaca
10261 ttctatgcaa aattgtgtac ttaagcttaa ggttgataca gccaatccta agacacctaa
10321 gtataagttt gttcgcattc aaccaggaca gactttttca gtgttagctt gttacaatgg
10381 ttcaccatct ggtgtttacc aatgtgctat gaggcccaat ttcactatta agggttcatt
10441 ccttaatggt tcatgtggta gtgttggttt taacatagat tatgactgtg tctctttttg
10501 ttacatgcac catatggaat taccaactgg agttcatgct ggcacagact tagaaggtaa
10561 cttttatgga ccttttgttg acaggcaaac agcacaagca gctggtacgg acacaactat
10621 tacagttaat gttttagctt ggttgtacgc tgctgttata aatggagaca ggtggtttct
10681 caatcgattt accacaactc ttaatgactt taaccttgtg gctatgaagt acaattatga
10741 acctctaaca caagaccatg ttgacatact aggacctctt tctgctcaaa ctggaattgc
10801 cgttttagat atgtgtgctt cattaaaaga attactgcaa aatggtatga atggacgtac
10861 catattgggt agtgctttat tagaagatga atttacacct tttgatgttg ttagacaatg
10921 ctcaggtgtt actttccaaa gtgcagtgaa aagaacaatc aagggtacac accactggtt
10981 gttactcaca attttgactt cacttttagt tttagtccag agtactcaat ggtctttgtt
11041 cttttttttg tatgaaaatg cctttttacc ttttgctatg ggtattattg ctatgtctgc
11101 ttttgcaatg atgtttgtca aacataagca tgcatttctc tgtttgtttt tgttaccttc
11161 tcttgccgct gtagcttatt ttaatatggt ctatatgcct gctagttggg tgatgcgtat
11221 tatgacatgg ttggatatgg ttgatactag tttgtctggt tttaagctaa aagactgtgt
11281 tatgtatgca tcagctgtgg tgttactaat ccttatgaca gcaagaactg tgtatgatga
11341 tggtgctagg agag
            [gap 482 bp] Expand Ns
11837 tcag tagtcttact ctcagttttg caacaactca gagtagaatc
11881 atcatctaaa ttgtgggctc aatgtgtcca gttacacaat gacattctct tagctaaaga
11941 tactactgaa gcctttgaaa aaatggtttc actactttct gttttgcttt ccatgcaggg
12001 tgctgtagac ataaacaagc tttgtgaaga aatgctggac aacagggcaa ccttacaagc
12061 tatagcctca gagtttagtt cccttccatc atatgcagct tttgctactg ctcaagaagc
12121 ttatgagcag gctgttgcta atggtgattc tgaagttgtt cttaaaaagt tgaagaagtc
12181 tttgaatgtg gctaaatctg aatttgaccg tgatgcagcc atgcaacgta agttggaaaa
12241 gatggctgat caagctatga cccaaatgta taaacaggct agatctgagg acaagagggc
12301 aaaagttact agtgctatgc agacaatgct tttcactatg cttagaaagt tggataatga
12361 tgcactcaac aacattatca acaatgcaag agatggttgt gttcccttga acataatacc
12421 tcttacaaca gcagccaaac taatggttgt cataccagac tataacacat ataaaaatac
12481 gtgtgatggt acaacattta cttatgcatc agcattgtgg gaaatccaac aggttgtaga
12541 tgcagatagt aaaattgttc aacttagtga aattagtatg gacaattcac ctaatttagc
12601 atggcctctt attgtaacag ctttaagggc caattctgct gtcaaattac agaataatga
12661 gcttagtcct gttgcactac gacagatgtc ttgtgctgcc ggtactacac aaactgcttg
12721 cactgatgac aatgcgttag cttactacaa cacaacaaag ggaggtaggt ttgtacttgc
12781 actgttatcc gatttacagg atttgaaatg ggctagattc cctaagagtg atggaactgg
12841 tactatctat acagaactgg aaccaccttg taggtttgtt acagacacac ctaaaggtcc
12901 taaagtgaag tatttatact ttattaaagg attaaacaac ctaaatagag gtatggtact
12961 tggtagttta gctgccacag tacgtctaca agctggtaat gcaacagaag tgcctgccaa
13021 ttcaactgta ttatctttct gtgcttttgc tgtagatgct gctaaagctt acaaagatta
13081 tctagctagt gggggacaac caatcactaa ttgtgttaag atgttgtgta cacacactgg
13141 tactggtcag gcaataacag ttacaccgga agccaatatg gatcaagaat cctttggtgg
13201 tgcatcgtgt tgtctgtact gccgttgcca catagatcat ccaaatccta aaggattttg
13261 tgacttaaaa ggtaagtatg tacaaatacc tacaacttgt gctaatgacc ctgtgggttt
13321 tacacttaaa aacacagtct gtaccgtctg cggtatgtgg aaaggttatg gctgtagttg
13381 tgatcaactc cgcgaaccca tgcttcagtc agctgatgca caatcgtttt taaacgggtt
13441 tgcggtgtaa gtgcagcccg tcttacaccg tgcggcacag gcactagtac tgatgtcgta
13501 tacagggctt ttgacatcta caatgataaa gtagctggtt ttgctaaatt cctaaaaact
13561 aattgttgtc gcttccaaga aaaggacgaa gatgacaatt taattgattc ttactttgta
13621 gttaagagac acactttctc taactaccaa catgaagaaa caatttataa tttacttaag
13681 gattgtccag ctgttgctaa acatgacttc tttaagttta gaatagacgg tgacatggta
13741 ccacatatat cacgtcaacg tcttactaaa tacacaatgg cagacctcgt ctatgcttta
13801 aggcattttg atgaaggtaa ttgtgacaca ttaaaagaaa tacttgtcac atacaattgt
13861 tgtgatgatg attatttcaa taaaaaggac tggtatgatt ttgtagaaaa cccagatata
13921 ttacgcgtat acgccaactt aggtgaacgt gtacgccaag ctttgttaaa aacagtacaa
13981 ttctgtgatg ccatgcgaaa tgctggtatt gttggtgtac tgacattaga taatcaagat
14041 ctcaatggta actggtatga tttcggtgat ttcatacaaa ccacgccagg tagtggagtt
14101 cctgttgtag attcttatta ttcattgtta atgcctatat taaccttgac cagggcttta
14161 actgcagagt cacatgttga cactgactta acaaagcctt acattaagtg ggatttgtta
14221 aaatatgact tcacggaaga gaggttaaaa ctctttgacc gttattttaa atattgggat
14281 cagacatacc acccaaattg tgttaactgt ttggatgaca gatgcattct gcattgtgca
14341 aactttaatg ttttattctc tacagtgttc ccacttacaa gttttggacc actagtgaga
14401 aaaatatttg ttgatggtgt tccatttgta gtttcaactg gataccactt cagagagcta
14461 ggtgttgtac ataatcagga tgtaaactta catagctcta gacttagttt taaggaatta
14521 cttgtgtatg ctgctgaccc tgctatgcac gctgcttctg gtaatctatt actagataaa
14581 cgcactacgt gcttttcagt agctgcactt actaacaatg ttgcttttca aactgtcaaa
14641 cccggtaatt ttaacaaaga cttctatgac tttgctgtgt ctaagggttt ctttaaggaa
14701 ggaagttctg ttgaattaaa acacttcttc tttgctcagg atggtaatgc tgctatcagc
14761 gattatgact actatcgtta taatctacca acaatgtgtg atatcagaca actactattt
14821 gtagttgaag ttgttgataa gtactttgat tgttacgatg gtggctgtat taatgctaac
14881 caagtcatcg tcaacaacct agacaaatca gctggttttc catttaataa atggggtaag
14941 gctagacttt attatgattc aatgagttat gaggatcaag atgcactttt cgcatataca
15001 aaacgtaatg tcatccctac tataactcaa atgaatctta agtatgccat tagtgcaaag
15061 aatagagctc gcaccgtagc tggtgtctct atctgtagta ctatgaccaa tagacagttt
15121 catcaaaaat tattgaaatc aatagccgcc actagaggag ctactgtagt aattggaaca
15181 agcaaattct atggtggttg gcacaacatg ttaaaaactg tttatagtga tgtagaaaac
15241 cctcacctta tgggttggga ttatcctaaa tgtgatagag ccatgcctaa catgcttaga
15301 attatggcct cacttgttct tgctcgcaaa catacaacgt gttgtagctt gtcacaccgt
15361 ttctatagat tagctaatga gtgtgctcaa gtattgagtg aaatggtcat gtgtggcagt
15421 tcactatatg ttaaaccagg tggaacctca tcaggagatg ccacaactgc ttatgctaat
15481 agtgttttta acatttgtca agctgtcacg gccaatgtta atgcactttt atctactgat
15541 ggtaacaaaa ttgccgataa gtatgtccgc aatttacaac acagacttta tgagtgtctc
15601 tatagaaata gagatgttga cacagacttt gtgaatgagt tttacgcata tttgcgtaaa
15661 catttctcaa tgatgatact ctctgacgat gctgttgtgt gtttcaatag cacttatgca
15721 tctcaaggtc tagtggctag cataaagaac tttaagtcag ttctttatta tcaaaacaat
15781 gtttttatgt ctgaagcaaa atgttggact gagactgacc ttactaaagg acctcatgaa
15841 ttttgctctc aacatacaat gctagttaaa cagggtgatg attatgtgta ccttccttac
15901 ccagatccat caagaatcct aggggccggc tgttttgtag atgatatcgt aaaaacagat
15961 ggtacactta tgattgaacg gttcgtgtct ttagctatag atgcttaccc acttactaaa
16021 catcctaatc aggagtatgc tgatgtcttt catttgtact tacaatacat aagaaagcta
16081 catgatgagt taacaggaca catgttagac atgtattctg ttatgcttac taatgataac
16141 acttcaaggt attgggaacc tgagttttat gaggctatgt acacaccgca tacagtctta
16201 caggctgttg gggcttgtgt tctttgcaat tcacagactt cattaagatg tggtgcttgc
16261 atacgtagac cattcttatg ttgtaaatgc tgttacgacc atgtcatatc aacatcacat
16321 aaattagtct tgtctgttaa tccgtatgtt tgcaatgctc caggttgtga tgtcacagat
16381 gtgactcaac tttacttagg aggtatgagc tattattgta aatcacataa actacccatt
16441 agttttccat tgtgtgctaa tggacaagtt tttggtttat ataaaaatac atgtgttggt
16501 agcgataatg ttactgactt taatgcaatt gcaacatgtg actggacaaa tgctggtgat
16561 tacattttag ctaacacctg tactgaaaga ctcaagcttt ttgcagcaga aacgctcaaa
16621 gctactgagg agacatttaa actgtcttat ggtattgcta ctgtacgtga agtgctgtct
16681 gacagagaat tacatctttc atgggaagtt ggtaaaccta gaccaccact taaccgaaat
16741 tatgtcttta ctggttatcg tgtaactaaa aacagtaaag tacaaatagg agagtacacc
16801 tttgaaaaag gtgactatgg tgatgctgtt gtttaccgag gtacaacaac ttacaaatta
16861 aatgttggtg attattttgt tctgacatca catacagtaa tgccattaag tgcacctaca
16921 ctagtgccac aagagcacta tgttagaatt actggcttat acccaacact caatatctca
16981 gatgagtttt ctagcaatgt tgcaaattat caaaaggttg gtatgcaaaa gtattctaca
17041 ctccagggac cacctggtac tggtaagagt cattttgcta ttggcctagc tctctactac
17101 ccttctgctc gcatagtgta tacagcttgc tctcatgccg ctgttgatgc actatgtgag
17161 aaggcattaa aatatttgcc tatagataaa tgtagtagaa ttatacctgc acgtgctcgt
17221 gtagagtgtt ttgataaatt caaagtgaat tcaacattag aacagtatgt cttttgtact
17281 gtaaatgcat tgcctgagac gacagcagat atagttgtct ttgatgaaat ttcaatggcc
17341 acaaattatg atttgagtgt tgtcaatgcc agattacgtg ctaagcacta tgtgtacatt
17401 ggcgaccctg ctcaattacc tgcaccacgc acattgctaa ctaagggcac actagaacca
17461 gaatatttca attcagtgtg tagacttatg aaaactatag gtccagacat gttcctcgga
17521 acttgtcggc gttgtcctgc tgaaattgtt gacactgtga gtgctttggt ttatgataat
17581 aagcttaaag cacataaaga caaatcagct caatgcttta aaatgtttta taagggtgtt
17641 atcacgcatg atgtttcatc tgcaattaac aggccacaaa taggcgtggt aagagaattc
17701 cttacacgta acccagcttg gagaaaagct gtctttattt caccttataa ttcacagaat
17761 gctgtagcct caaagatttt gggactacca actcaaactg ttgattcatc acagggctca
17821 gaatatgact atgtcatatt cactcaaacc actgaaacag ctcactcttg taatgtaaac
17881 agatttaatg ttgctattac cagagcaaaa gtaggcatac tttgcataat gtctgataga
17941 gacctttatg acaagttgca atttacaagt cttgaaattc cacgtaggaa tgtggcaact
18001 ttacaagctg aaaatgtaac aggactcttt aaagattgta gtaaggtaat cactgggtta
18061 catcctacac aggcacctac acacctcagt gttgacacta aattcaaaac tgaaggttta
18121 tgtgttgaca tacctggcat acctaaggac atgacctata gaagactcat ctctatgatg
18181 ggttttaaaa tgaattatca agttaatggt taccctaaca tgtttatcac ccgcgaagaa
18241 gctataagac atgtacgtgc atggattggc ttcgatgtcg aggggtgtca tgctactaga
18301 gaagctgttg gtaccaattt acctttacag ctaggttttt ctacaggtgt taacctagtt
18361 gctgtaccta caggttatgt tgatacacct aataatacag atttttccag agttagtgct
18421 aaaccaccgc ctggagatca atttaaacac ctcataccac ttatgtacaa aggacttcct
18481 tggaatgtag tgcgtataaa gattgtacaa atgttaagtg acacacttaa aaatctctct
18541 gacagagtcg tatttgtctt atgggcacat ggctttgagt tgacatctat gaagtatttt
18601 gtgaaaatag gacctgagcg cacctgttgt ctatgtgata gacgtgccac atgcttttcc
18661 actgcttcag acacttatgc ctgttggcat cattctattg gatttgatta cgtctataat
18721 ccgtttatga ttgatgttca acaatggggt tttacaggta acctacaaag caaccatgat
18781 ctgtattgtc aagtccatgg taatgcacat gtagctagtt gtgatgcaat catgactagg
18841 tgtctagctg tccacgagtg ctttgttaag cgtgttgact ggactattga atatcctata
18901 attggtgatg aactgaagat taatgcggct tgtagaaagg ttcaacacat ggttgttaaa
18961 gctgcattat tagcagacaa attcccagtt cttcacgaca ttggtaaccc taaagctatt
19021 aagtgtgtac ctcaagctta tgtagaatgg aagttctatg atgcacagcc ttgtagtgac
19081 aaagcttata aaatagaaga attattctat tcttatgcca cacattctga caaattcaca
19141 gatggtgtat gcctattttg gaattgcaat gtcgatagat atcctgttaa ttccattgtt
19201 tgtagatttg acactagagt gctatctaac cttaacttgc ctggttgtga tggtggcagt
19261 ttgtatgtaa ataaacatgc attccacaca ccagcttttg ataaaagtgc ttttgttaat
19321 ttaaaacaat taccattttt ctattactct gacagtccat gtgagtctca tggaaaacaa
19381 gtagtgtcag atatagatta tgtaccacta aagtctgcta cgtgtataac acgttgcaat
19441 ttaggtggtg ctgtctgtag acatcatgct aatgagtaca gattgtatct cgatgcttat
19501 aacatgatga tctcagctgg ctttagcttg tgggtttaca aacaatttga tacttataac
19561 ctctggaaca cttttacaag acttcagagt ttagaaaatg tggcttttaa tgttgtaaat
19621 aagggacact ttgatggaca acagggtgaa gtaccagttt ctatcattaa taacactgtt
19681 tacacaaaag ttgatggtgt tgatgtagaa ttgtttgaaa ataaaacaac attacctgtt
19741 aatgtagcat ttgagctttg ggctaagcgc aacattaaac cagtaccaga ggtgaaaata
19801 ctcaataatt tgggtgtgga cattgctgct aatactgtga tctgggacta caaaagagat
19861 gctccagcac atatatctac tattggtgtt tgttctatga ctgacatagc caagaaacca
19921 actgaaacga tttgtgcacc actcactgtc ttttttgatg gtagagttga tggtcaagta
19981 gacttattta gaaatgcccg taatggtgtt cttattacag aaggtagtgt taaaggttta
20041 caaccatctg taggtcccaa acaagctagt cttaatggag tcacattaat tggagaagcc
20101 gtaaaaacac agttcaatta ttataagaaa gttgatggtg ttgtccaaca attacctgaa
20161 acttacttta ctcagagtag aaatttacaa gaatttaaac ccaggagtca aatggaaatt
20221 gatttcttag aattagctat ggatgaattc attgaacggt ataaattaga aggctatgcc
20281 ttcgaacata tcgtttatgg agattttagt catagtcagt taggtggttt acatctactg
20341 attggactag ctaaacgttt taaggaatca ccttttgaat tagaagattt tattcctatg
20401 gacagtacag ttaaaaacta tttcataaca gatgcgcaaa caggttcatc taagtgtgtg
20461 tgttctgtta ttgatttatt acttgatgat tttgttgaaa taataaaatc ccaagattta
20521 tctgtagttt ctaaggttgt caaagtgact attgactata cagaaatttc atttatgctt
20581 tggtgtaaag atggccatgt agaaacattt tacccaaaat tacaatctag tcaagcgtgg
20641 caaccgggtg ttgctatgcc taatctttac aaaatgcaaa gaatgctatt agaaaagtgt
20701 gaccttcaaa attatggtga tagtgcaaca ttacctaaag gcataatgat gaatgtcgca
20761 aaatatactc aactgtgtca atatttaaac acattaacat tagctgtacc ctataatatg
20821 agagttatac attttggtgc tggttctgat aaaggagttg caccaggtac agctgtttta
20881 agacagtggt tgcctacggg tacgctgctt gtcgattcag atcttaatga ctttgtctct
20941 gatgcagatt caactttgat tggtgattgt gcaactgtac atacagctaa taaatgggat
21001 ctcattatta gtgatatgta cgaccctaag actaaaaatg ttacaaaaga aaatgactct
21061 aaagagggtt ttttcactta catttgtggg tttatacaac aaaagctagc tcttggaggt
21121 tccgtggcta taaagataac agaacattct tggaatgctg atctttataa gctcatggga
21181 cacttcgcat ggtggacagc ctttgttact aatgtgaatg cgtcatcatc tgaagcattt
21241 ttaattggat gtaattatct tggcaaacca cgcgaacaaa tagatggtta tgtcatgcat
21301 gcaaattaca tattttggag gaatacaaat ccaattcagt tgtcttccta ttctttattt
21361 gacatgagta aatttcccct taaattaagg ggtactgctg ttatgtcttt aaaagaaggt
21421 caaatcaatg atatgatttt atctcttctt agtaaaggta gacttataat tagagaaaac
21481 aacagagttg ttatttctag tgatgttctt gttaacaact aaacgaacaa tgtttgtttt
21541 tcttgtttta ttgccactag tctctagtca gtgtgttaat cttagaacca gaactcaatt
21601 accccctgca tacactaatt ctttcacacg tggtgtttat taccctgaca aagttttcag
21661 atcctcagtt ttacattcaa ctcaggactt gttcttacct
            [gap 257 bp] Expand Ns
21958 tta
21961 ttaccacaaa aacaacaaaa gttggatgga aagtggagtt tattctagtg cgaataattg
22021 cacttttgaa tatgtctctc agccttttct tatggacctt gaaggaaaac agggtaattt
22081 caaaaatctt agggaatttg tgtttaagaa tattgatggt tattttaaaa tatattctaa
22141 gcacacgcct attaatttag tgcgtgatct ccctcagggt ttttcggctt tagaaccatt
22201 ggtagatttg ccaataggta ttaaca
            [gap 251 bp] Expand Ns
22478 aga gtccaaccaa cagaatctat
22501 tgttagattt cctaatatta caaacttgtg cccttttggt gaagttttta acgccaccag
22561 atttgcatct gtttatgctt ggaacaggaa gagaatcagc aactgtgttg ctgattattc
22621 tgtcctatat aattccgcat cattttccac ttttaagtgt tatggagtgt ctcctactaa
22681 attaaatgat ctctgcttta ctaatgtcta tgcagattca tttgtaatta gaggtgatga
22741 agtcagacaa atcgctccag ggcaaactgg aaagattgct gattataatt ataaattacc
22801 agatgatttt acaggctgcg ttatagcttg gaattctaac aatcttgatt ctaaggttgg
22861 tggtaattat aattaccggt atagattgtt taggaagtct aatctcaaac cttttgagag
22921 agatatttca actgaaatct atcaggccgg tagcaaacct tgtaatggtg ttgaaggttt
22981 taattgttac tttcctttac aatcatatgg tttccaaccc actaatggtg ttggttacca
23041 accatacaga gtagtagtac tttcttttga acttctacat gcaccagcaa ctgtttgtgg
23101 acctaaaaag tctactaatt tggttaaaaa caaatgtgtc aatttcaact tcaatggttt
23161 aacaggcaca ggtgttctta ctgagtctaa caaaaagttt ctgcctttcc aacaatttgg
23221 cagagacatt gctgacacta ctgatgctgt ccgtgatcca cagacacttg agattcttga
23281 cattacacca tgttcttttg gtggtgtcag tgttataaca ccaggaacaa atacttctaa
23341 ccaggttgct gttctttatc agggtgttaa ctgcacagaa gtccctgttg ctattcatgc
23401 agatcaactt actcctactt ggcgtgttta ttctacaggt tctaatgttt ttcaaacacg
23461 tgcaggctgt ttaatagggg ctgaacatgt caacaactca tatgagtgtg acatacccat
23521 tggtgcaggt atatgcgcta gttatcagac tcagactaat tctcgtcggc gggcacgtag
23581 tgtagctagt caatccatca ttgcctacac tatgtcactt ggtgcagaaa attcagttgc
23641 ttactctaat aactctattg ccatacccac aaattttact attagtgtta ccacagaaat
23701 tctaccagtg tctatgacca agacatcagt agattgtaca atgtacattt gtggtgattc
23761 aactgaatgc agcaatcttt tgttgcaata tggcagtttt tgtacacaat taaaccgtgc
23821 tttaactgga atagctgttg aacaagacaa aaacacccaa gaagtttttg cacaagtcaa
23881 acaaatttac aaaacaccac caattaaaga ttttggtggt tttaattttt cacaaatatt
23941 accagatcca tcaaaaccaa gcaagaggtc atttattgaa gatctacttt tcaacaaagt
24001 gacacttgca gatgctggct tcatcaaaca atatggtgat tgccttggtg atattgctgc
24061 tagagacctc atttgtgcac aaaagtttaa cggccttact gttttgccac ctttgctcac
24121 agatgaaatg attgctcaat acacttctgc actgttagcg ggtacaatca cttctggttg
24181 gacctttggt gcaggtgctg cattacaaat accatttgct atgcaaatgg cttataggtt
24241 taatggtatt ggagttacac agaatgttct ctatgagaac caaaaattga ttgccaacca
24301 atttaatagt gctattggca aaattcaaga ctcactttct tccacagcaa gtgcacttgg
24361 aaaacttcaa aatgtggtca accaaaatgc acaagcttta aacacgcttg ttaaacaact
24421 tagctccaat tttggtgcaa tttcaagtgt tttaaatgat atcctttcac gtcttgacaa
24481 agttgaggct gaagtgcaaa ttgataggtt gatcacaggc agacttcaaa gtttgcagac
24541 atatgtgact caacaattaa ttagagctgc agaaatcaga gcttctgcta atcttgctgc
24601 tactaaaatg tcagagtgtg tacttggaca atcaaaaaga gttgattttt gtggaaaggg
24661 ctatcatctt atgtccttcc ctcagtcagc acctcatggt gtagtcttct tgcatgtgac
24721 ttatgtccct gcacaagaaa agaacttcac aactgctcct gccatttgtc atgatggaaa
24781 agcacacttt cctcgtgaag gtgtctttgt ttcaaatggc acacactggt ttgtaacaca
24841 aaggaatttt tatgaaccac aaatcattac tacagacaac acatttgtgt ctggtaactg
24901 tgatgttgta ataggaattg tcaacaacac agtttatgat cctttgcaac ctgaattaga
24961 ctcattcaag gaggagttag ataaatattt taagaatcat acatcaccag atgttgattt
25021 aggtgacatc tctggcatta atgcttcagt tgtaaacatt caaaaagaaa ttgaccgcct
25081 caatgaggtt gccaagaatt taaatgaatc tctcatcgat ctccaagaac ttggaaagta
25141 tgagcagtat ataaaatggc catggtacat ttggctaggt tttatagctg gcttgattgc
25201 catagtaatg gtgacaatta tgctttgctg tatgaccagt tgctgtagtt gtctcaaggg
25261 ctgttgttct tgtggatcct gctgcaaatt tgatgaagac gactctgagc cagtgctcaa
25321 aggagtcaaa ttacattaca cataaacgaa cttatggatt tgtttatgag aatcttcaca
25381 attggaactg taactttgaa gcaaggtgaa atcaaggatg ctactccttt agattttgtt
25441 cgcgctactg caacgatacc gatacaagcc tcactccctt tcggatggct tattgttggc
25501 gttgcacttc ttgctgtttt tcagagcgct tccaaaatca taaccctcaa aaagagatgg
25561 caactagcac tctccaaggg tgttcacttt gtttgcaact tgctgttgtt gtttgtaaca
25621 gtttactcac accttttgct cgttgctgct ggccttgaag ccccttttct ctatctttat
25681 gctttagtct acttcttgca gagtataaac tttgtaagaa taataatgag gctttggctt
25741 tgctggaaat gccgttccaa aaacccatta ttttatgatg ccaactattt ttttgctgg
25801 catactaatt gttacgacta ttgtatacct tacaatagtg taacttcttc aattgtcatt
25861 acttcaggtg atggcacaac aagtcctatt tctgaacatg actaccagat tggtggttat
25921 actgaaaaat gggaatctgg agtaaaagac tgtgttgtat tacacagtta cttcacttca
25981 gactattacc agctgtactc aactcaattg agtacagaca ctggtgttga acatgttacc
26041 ttcttcatct acaataaaat tgttgatgag cctgaagaac atgtccaaat tcacacaatc
26101 gacggttcat ccggagttgt taatccagta atggaaccaa tttatgatga accgacgacg
26161 actactagcg tgcctttgta agcacaagct gatgagtacg aacttatgta ctcattcgtt
26221 tcggaagaga caggtacgtt aatagttaat agcgtacttc tttttcttgc tttcgtggta
26281 ttcttgctag ttacactagc catccttact gcgcttcgat tgtgtgcgta ctgctgcaat
26341 attgttaacg tgagtcttgt aaaaccttct ttttacgttt actctcgttt taaaaatctg
26401 aattcttcta gagttcctga tcttctggtc taaacgaact aaatattata ttagtttttc
26461 tgtttggaac tttaatttta gccatggcag attccaacgg tactattacc gttgaagagc
26521 ttaaaaagct ccttgaacaa tggaacctag taataggttt cctattcctt acatggattt
26581 gtcttctaca atttgcctat gccaacagga ataggttttt gtatataatt aagttaattt
26641 tcctctggct gttatggcca gtaactttag cttgttttgt gcttgctgct gtttacagaa
26701 taaattggat caccggtgga attgctaccg caatggcttg tcttgtaggc ttgatgtggc
26761 tcagctactt cattgcttct ttcagactgt ttgcgcgtac gcgttccatg tggtcattca
26821 atccagaaac taatattctt ctcaacgtgc cactccatgg cactattctg accagaccgc
26881 ttctagaaag tgaactcgta atcggagctg tgatccttcg tggacatctt cgtattgctg
26941 gacaccatct aggacgctgt gacatcaagg acctgcctaa agaaatcact gttgctacat
27001 cacgaacgct ttcttattac aaattgggag cttcgcagcg tgtagcaggt gactcaggtt
27061 ttgctgcata cagtcgctac aggattggca actataaatt aaacacagac cattccagta
27121 gcagtgacaa tattgctttg cttgtacagt aagtgacaac agatgtttca tctcgttgac
27181 tttcaggtta ctatagcaga gatattacta attattatga ggacttttaa agtttccatt
27241 tggaatcttg attacatcat aaacctcata attaaaaatt tatctaagtc actaactgag
27301 aataaatatt ctcaattaga tgaagagcaa ccaatggaga ttgattaaac gaacatgaaa
27361 attattcttt tcttggcact gataacactc gctacttgtg agctttatca ctaccaagag
27421 tgtgttagag gtacaacagt acttttaaaa gaaccttgct cttctggaac atacgagggc
27481 aattcaccat ttcatcctct agctgataac aaatttgcac tgacttgctt tagcactcaa
27541 tttgcttttg cttgtcctga cggcgtaaaa cacgtctatc agttacgtgc cagatcagct
27601 tcacctaaac tgttcatcag acaagaggaa gttcaagaac tttactctcc aatttttctt
27661 attgttgcgg caatagtgtt tataacactt tgcttcacac tcaaaagaaa gatagaatga
27721 ttgaactttc attaattgac ttctatttgt gctttttagc ctttctgcta ttccttgttt
27781 taattatgct tattatcttt tggttctcac ttgaactgca agatcataat gaaatttgtc
27841 acgcctaaac gaacatgaaa tttcttgttt tcttaggaat catcacaact gtagctgcat
27901 ttcaccaaga atgtagttta cagtcatgta ctcaacatca accatatgta gttgatgacc
27961 cgtgtcctat tcacttctat tctaaatggt atattagagt aggagctaga aaatcagcac
28021 ctttaattga attgtgcgtg gatgaggctg gttctaaatc acccattcag tacatcgata
28081 tcggtaatta tacagtttcc tgtttacctt ttacaattaa ttgccaggaa cctaaattgg
28141 gtagtcttgt agtgcgttgt tcgttctatg aagacttttt agagtatcat gacgttcgtg
28201 ttgttttaat ctaaacgaac aaactaaatg tctgataatg gaccccaaaa tcagcgaaat
28261 gcaccccgca ttacgtttgg tggaccctca gattcaactg gcagtaacca gaatggagaa
28321 cgcagtgggg cgcgatcaaa acaacgtcgg ccccaaggtt tacccaataa tactgcgtct
28381 tggttcaccg ctctcactca acatggcaag gaaggcctta aattccctcg aggacaaggc
28441 gttccaatta acaccaatag cagtccagat gaccaaattg gctactaccg aagagctacc
28501 agacgaattc gtggtggtga cggtaaaatg aaagatctca gtccaagatg gtatttctac
28561 tacctaggaa ctgggccaga agctggactt ccctatggtg ctaacaaaga cggcatcata
28621 tgggttgcaa ctgagggagc cttgaataca ccaaaagatc acattggcac ccgcaatcct
28681 gctaacaatg ctgcaatcgt gctacaactt cctcaaggaa caacattgcc aaaaggcttc
28741 tacgcagaag ggagcagagg cggcagtcaa gcctcttctc gttcctcatc acgtagtcgc
28801 aacagttcaa gaaattcaac tccaggcagc agtatgggaa cttctcctgc tagaatggct
28861 ggcaatggct gtgatgctgc tcttgctttg ctgctgcttg acagattgaa ccagcttgag
28921 agcaaaatgt ctggtaaagg ccaacaacaa caaggccaaa ctgtcactaa gaaatctgct
28981 gctgaggctt ctaagaagcc tcggcaaaaa cgtactgcca ctaaagcata caatgtaaca
29041 caagctttcg gcagacgtgg tccagaacaa acccaaggaa attttgggga ccaggaacta
29101 atcagacaag gaactgatta caaacattgg ccgcaaattg cacaatttgc ccccagcgct
29161 tcagcgttct tcggaatgtc gcgcattggc atggaagtca caccttcggg aacgtggttg
29221 acctacacag gtgccatcaa attggatgac aaagatccaa atttcaaaga tcaagtcatt
29281 ttgctgaata agcatattga cgcatacaaa acattcccac caacagagcc taaaaaggac
29341 aaaaagaaga aggcttatga aactcaagcc ttaccgcaga gacagaagaa acagcaaact
29401 gtgactcttc ttcctgctgc agatttggat gatttctcca aacaattgca acaatccatg
29461 agcagtgctg actcaactca ggcctaaact catgcagacc acacaaggca gatgggctat
29521 ataaacgttt tcgcttttcc gtttacgata tatagtctac tcttgtgcag aatgaattct
29581 cgtaactaca tagcacaagt agatgtagtt aactttaatc tcacatagca atctttaatc
29641 agtgtgtaac attagggagg acttgaaaga gccaccacat tttcaccgag gccactcgga
29701 gtacgatcga gtgtacagtg aacaatgcta gggagagctg cctatatgga agagccctaa
29761 tgtgtaaaat taattttagt agtgctatcc ccatgtgatt ttaatagctn nnnnnnnnnn
29821 nnnnaaaaaa aaaaaaaaaa aaaaaaaaaa aaaaaaa
Omicrcn: Genbank OM011974.1
Nucleic Acid Sequence
(SEQ ID NO: 8)
        1 aacaaaccaa ccaactttcg atctcttgta gatctgttct ctaaacgaac tttaaaatct
      61 gtgtggctgt cactcggctg catgettagt gcactcacgc agtataatta ataactaatt
    121 actgtcgttg acaggacacg agtaactcgt ctatcttctg caggctgctt acggtttcgt
    181 ccgtgttgca gccgatcatc agcacatcta ggttttgtcc gggtgtgacc gaaaggtaag
    241 atggagagcc ttgtccctgg tttcaacgag aaaacacacg tccaactcag tttgcctgtt
    301 ttacaggttc gcgacgtgct cgtacgtggc tttggagact ccgtggagga ggtcttatca
    361 gaggcacgtc aacatcttaa agatggcact tgtggcttag tagaagttga aaaaggcgtt
    421 ttgcctcaac ttgaacagcc ctatgtgttc atcaaacgtt cggatgctcg aactgcacct
    481 catggtcatg ttatggttga gctggtagca gaactcgaag gcattcagta cggtcgtagt
    541 ggtgagacac ttggtgtcct tgtccctcat gtgggcgaaa taccagtggc ttaccgcaag
    601 gttcttcttc gtaagaacgg taataaagga gctggtggcc atagttacgg cgccgatcta
    661 aagtcatttg acttaggcga cgagcttggc actgatcctt atgaagattt tcaagaaaac
    721 tggaacacta aacatagcag tggtgttacc cgtgaactca tgcgtgagct taacggaggg
    781 gcatacactc gctatgtcga taacaacttc tgtggccctg atggctaccc tcttgagtgc
    841 attaaagacc ttctagcacg tgctggtaaa gcttcatgca ctttgtccga acaactggac
    901 tttattgaca ctaagagggg tgtatactgc tgccgtgaac atgagcatga aattgcttgg
    961 tacacggaac gttctgaaaa gagctatgaa ttgcagacac cttttgaaat taaattggca
  1021 aagaaatttg acaccttcaa tggggaatgt ccaaattttg tatttccctt aaattccata
  1081 atcaagacta ttcaaccaag ggttgaaaag aaaaagcttg atggctttat gggtagaatt
  1141 cgatctgtct atccagttgc gtcaccaaat gaatgcaacc aaatgtgcct ttcaactctc
  1201 atgaagtgtg atcattgtgg tgaaacttca tggcagacgg gcgattttgt taaagccact
  1261 tgcgaatttt gtggcactga gaatttgact aaagaaggtg ccactacttg tggttactta
  1321 ccccaaaatg ctgttgttaa aatttattgt ccagcatgtc acaattcaga agtaggacct
  1381 gagcatagtc ttgccgaata ccataatgaa tctggcttga aaaccattct tcgtaagggt
  1441 ggtcgcacta ttgcctttgg aggctgtgtg ttctcttatg ttggttgcca taacaagtgt
  1501 gcctattggg ttccacgtgc tagcgctaac ataggttgta accatacagg tgttgttgga
  1561 gaaggttccg aaggtcttaa tgacaacctt cttgaaatac tccaaaaaga gaaagtcaac
  1621 atcaatattg ttggtgactt taaacttaat gaagagatcg ccattatttt ggcatctttt
  1681 tctgcttcca caagtgcttt tgtggaaact gtgaaaggtt tggattataa agcattcaaa
  1741 caaattgttg aatcctgtgg taattttaaa gttacaaaag gaaaagctaa aaaaggtgcc
  1801 tggaatattg gtgaacagaa atcaatactg agtcctcttt atgcatttgc atcagaggct
  1861 gctcgtgttg tacgatcaat tttctcccgc actcttgaaa ctgctcaaaa ttctgtgcgt
  1921 gttttacaga aggccgctat aacaatacta gatggaattt cacagtattc actgagactc
  1981 attgatgcta tgatgttcac atctgatttg gctactaaca atctagttgt aatggcctac
  2041 attacaggtg gtgttgttca gttgacttcg cagtggctaa ctaacatctt tggcactgtt
  2101 tatgaaaaac tcaaacccgt ccttgattgg cttgaagaga agtttaagga aggtgtagag
  2161 tttcttagag acggttggga aattgttaaa tttatctcaa cctgtgcttg tgaaattgtc
  2221 ggtggacaaa ttgtcacctg tgcaaaggaa attaaggaga gtgttcagac attctttaag
  2281 cttgtaaata aatttttggc tttgtgtgct gactctatca ttattggtgg agctaaactt
  2341 aaagccttga atttaggtga aacatttgtc acgcactcaa agggattgta cagaaagtgt
  2401 gttaaatcca gagaagaaac tggcctactc atgcctctaa aagccccaaa agaaattatc
  2461 ttcttagagg gagaaacact tcccacagaa gtgttaacag aggaagttgt cttgaaaact
  2521 ggtgatttac aaccattaga acaacctact agtgaagctg ttgaagctcc attggttggt
  2581 acaccagttt gtattaacgg gcttatgttg ctcgaaatca aagacacaga aaagtactgt
  2641 gcccttgcac ctaatatgat ggtaacaaac aataccttca cactcaaagg cggtgcacca
  2701 acaaaggtta cttttggtga tgacactgtg atagaagtgc aaggttacaa gagtgtgaat
  2761 atcacttttg aacttgatga aaggattgat aaagtactta atgagaggtg ctctgcctat
  2821 acagttgaac tcggtacaga agtaaatgag ttcgcctgtg ttgtggcaga tgctgtcata
  2881 aaaactttgc aaccagtatc tgaattactt acaccactgg gcattgattt agatgagtgg
  2941 agtatggcta catactactt atttgatgag tctggtgagt ttaaattggc ttcacatatg
  3001 tattgttctt tttaccctcc agatgaggat gaagaagaag gtgattgtga agaagaagag
  3061 tttgagccat caactcaata tgagtatggt actgaagatg attaccaagg taaacctttg
  3121 gaatttggtg ccacttctgc tgctcttcaa cctgaagaag agcaagaaga agattggtta
  3181 gatgatgata gtcaacaaac tgttggtcaa caagacggca gtgaggacaa tcagacaact
  3241 actattcaaa caattgttga ggttcaacct caattagaga tggaacttac accagttgtt
  3301 cagactattg aagtgaatag ttttagtggt tatttaaaac ttactgacaa tgtatacatt
  3361 aaaaatgcag acattgtgga agaagctaaa aaggtaaaac caacagtggt tgttaatgca
  3421 gccaatgttt accttaaaca tggaggaggt gttgcaggag ccttaaataa ggctactaac
  3481 aatgccatgc aagttgaatc tgatgattac atagctacta atggaccact taaagtgggt
  3541 ggtagttgtg ttttaagcgg acacaatctt gctaaacact gtcttcatgt tgtcggccca
  3601 aatgttaaca aaggtgaaga cattcaactt cttaagagtg cttatgaaaa ttttaatcag
  3661 cacgaagttc tacttgcacc attattatca gctggtattt ttggtgctga ccctatacat
  3721 tctttaagag tttgtgtaga tactgttcgc acaaatgtct acttagctgt ctttgataaa
  3781 aatctctatg acaaacttgt ttcaagcttt ttggaaatga agagtgaaaa gcaagttgaa
  3841 caaaagatcg ctgagattcc taaagaggaa gttaagccat ttataactga aagtaaacct
  3901 tcagttgaac agagaaaaca agatgataag aaaatcaaag cttgtgttga agaagttaca
  3961 acaactctgg aagaaactaa gttcctcaca gaaaacttgt tactttatat tgacattaat
  4021 ggcaatcttc atccagattc tgccactctt gttagtgaca ttgacatcac tttcttaaag
  4081 aaagatgctc catatatagt gggtgatgtt gttcaagagg gtgttttaac tgctgtggtt
  4141 atacctacta aaaaggctgg tggcactact gaaatgctag cgaaagcttt gagaaaagtg
  4201 ccaacagaca attatataac cacttacccg ggtcagggtt taaatggtta cactgtagag
  4261 gaggcaaaga cagtgcttaa aaagtgtaaa agtgcctttt acattctacc atctattatc
  4321 tctaatgaga agcaagaaat tcttggaact gtttcttgga atttgcgaga aatgcttgca
  4381 catgcagaag aaacacgcaa attaatgcct gtctgtgtgg aaactaaagc catagtttca
  4441 actatacagc gtaaatataa gggtattaaa atacaagagg gtgtggttga ttatggtgct
  4501 agattttact tttacaccag taaaacaact gtagcgtcac ttatcaacac acttaacgat
  4561 ctaaatgaaa ctcttgttac aatgccactt ggctatgtaa cacatggctt aaatttggaa
  4621 gaagctgctc ggtatatgag atctctcaaa gtgccagcta cagtttctgt ttcttcacct
  4681 gatgctgtta cagcgtataa tggttatctt acttcttctt ctaaaacacc tgaagaacat
  4741 tttattgaaa ccatctcact tgctggttcc tataaagatt ggtcctattc tggacaatct
  4801 acacaactag gtatagaatt tcttaagaga ggtgataaaa gtgtatatta cactagtaat
  4861 cctaccacat tccacctaga tggtgaagtt atcacctttg acaatcttaa gacacttctt
  4921 tctttgagag aagtgaggac tattaaggtg tttacaacag tagacaacat taacctccac
  4981 acgcaagttg tggacatgtc aatgacatat ggacaacagt ttggtccaac ttatttggat
  5041 ggagctgatg ttactaaaat aaaacctcat aattcacatg aaggtaaaac attttatgtt
  5101 ttacctaatg atgacactct acgtgttgag gcttttgagt actaccacac aactgatcct
  5161 agttttctgg gtaggtacat gtcagcatta aatcacacta aaaagtggaa atacccacaa
  5221 gttaatggtt taacttctat taaatgggca gataacaact gttatcttgc cactgcattg
  5281 ttaacactcc aacaaataga gttgaagttt aatccacctg ctctacaaga tgcttattac
  5341 agagcaaggg ctggtgaagc ggctaacttt tgtgcactta tcttagccta ctgtaataag
  5401 acagtaggtg agttaggtga tgttagagaa acaatgagtt acttgtttca acatgccaat
  5461 ttagattctt gcaaaagagt cttgaacgtg gtgtgtaaaa cttgtggaca acagcagaca
  5521 acccttaagg gtgtagaagc tgttatgtac atgggcacac tttcttatga acaatttaag
  5581 aaaggtgttc agataccttg tacgtgtggt aaacaagcta caaaatatct agtacaacag
  5641 gagtcacctt ttgttatgat gtcagcacca cctgctcagt atgaacttaa gcatggtaca
  5701 tttacttgtg ctagtgagta cactggtaat taccagtgtg gtcactataa acatataact
  5761 tctaaagaaa ctttgtattg catagacggt gctttactta caaagtcctc agaatacaaa
  5821 ggtcctatta cggatgtttt ctacaaagaa aacagttaca caacaaccat aaaaccagtt
  5881 acttataaat tggatggtgt tgtttgtaca gaaattgacc ctaagttgga caattattat
  5941 aagaaagaca attcttattt cacagagcaa ccaattgatc ttgtaccaaa ccaaccatat
  6001 ccaaacgcaa gcttcgataa ttttaagttt gtatgtgata atatcaaatt tgctgatgat
  6061 ttaaaccagt taactggtta taagaaacct gcttcaagag agcttaaagt tacatttttc
  6121 cctgacttaa atggtgatgt ggtggctatt gattataaac actacacacc ctcttttaag
  6181 aaaggagcta aattgttaca taaacctatt gtttggcatg ttaacaatgc aactaataaa
  6241 gccacgtata aaccaaatac ctggtgtata cgttgtcttt ggagcacaaa accagttgaa
  6301 acatcaaatt cgtttgatgt actgaagtca gaggacgcgc agggaatgga taatcttgcc
  6361 tgcgaagatc taaaaccagt ctctgaagaa gtagtggaaa atcctaccat acagaaagac
  6421 gttcttgagt gtaatgtgaa aactaccgaa gttgtaggag acattatact taaaccagca
  6481 aataatataa aaattacaga agaggttggc cacacagatc taatggctgc ttatgtagac
  6541 aattctagtc ttactattaa gaaacctaat gaattatcta gagtattagg tttgaaaacc
  6601 cttgctactc atggtttagc tgctgttaat agtgtccctt gggatactat agctaattat
  6661 gctaagcctt ttcttaacaa agttgttagt acaactacta acatagttac acggtgttta
  6721 aaccgtgttt gtactaatta tatgccttat ttctttactt tattgctaca attgtgtact
  6781 tttactagaa gtacaaattc tagaattaaa gcatctatgc cgactactat agcaaagaat
  6841 actgttaaga gtgtcggtaa attttgtcta gaggcttcat ttaattattt gaagtcacct
  6901 aatttttcta aactgataaa tattataatt tggtttttac tattaagtgt ttgcctaggt
  6961 tctttaatct actcaaccgc tgctttaggt gttttaatgt ctaatttagg catgccttct
  7021 tactgtactg gttacagaga aggctatttg aactctacta atgtcactat tgcaacctac
  7081 tgtactggtt ctataccttg tagtgtttgt cttagtggtt tagattcttt agacacctat
  7141 ccttctttag aaactataca aattaccatt tcatctttta aatgggattt aactgctttt
  7201 ggcttagttg cagagtggtt tttggcatat attcttttca ctaggttttt ctatgtactt
  7261 ggattggctg caatcatgca attgtttttc agctattttg cagtacattt tattagtaat
  7321 tcttggctta tgtggttaat aattaatctt gtacaaatgg ccccgatttc agctatggtt
  7381 agaatgtaca tcttctttgc atcattttat tatgtatgga aaagttatgt gcatgttgta
  7441 gacggttgta attcatcaac ttgtatgatg tgttacaaac gtaatagagc aacaagagtc
  7501 gaatgtacaa ctattgttaa tggtgttaga aggtcctttt atgtctatgc taatggaggt
  7561 aaaggctttt gcaaactaca caattggaat tgtgttaatt gtgatacatt ctgtgctggt
  7621 agtacattta ttagtgatga agttgcgaga gacttgtcac tacagtttaa aagaccaata
  7681 aatcctactg accagtcttc ttacatcgtt gatagtgtta cagtgaagaa tggttccatc
  7741 catctttact ttgataaagc tggtcaaaag acttatgaaa gacattctct ctctcatttt
  7801 gttaacttag acaacctgag agctaataac actaaaggtt cattgcctat taatgttata
  7861 gtttttgatg gtaagtcaaa atgtgaagaa tcatctgcaa aatcagcgtc tgtttactac
  7921 agtcagctta tgtgtcaacc tatactgtta ctagatcagg cattagtgtc tgatgttggt
  7981 gatagtgcgg aagttgcagt taaaatgttt gatgcttacg ttaatacgtt ttcatcaact
  8041 tttaacgtac caatggaaaa actcaaaaca ctagttgcaa ctgcagaagc tgaacttgca
  8101 aagaatgtgt ccttagacaa tgtcttatct acttttattt cagcagctcg gcaagggttt
  8161 gttgattcag atgtagaaac taaagatgtt gttgaatgtc ttaaattgtc acatcaatct
  8221 gacatagaag ttactggcga tagttgtaat aactatatgc tcacctataa caaagttgaa
  8281 aacatgacac cccgtgacct tggtgcttgt attgactgta gtgcgcgtca tattaatgcg
  8341 caggtagcaa aaagtcacaa cattactttg atatggaacg ttaaagattt catgtcattg
  8401 tctgaacaac tacgaaaaca aatacgtagt gctgctaaaa agaataactt accttttaag
  8461 ttgacatgtg caactactag acaagttgtt aatgttgtaa caacaaagat agcacttaag
  8521 ggtggtaaaa ttgttaataa ttggttgaag cagttaatta aagttatact tgtgttcctt
  8581 tttgttgctg ctattttcta tttaataaca cctgttcatg tcatgtctaa acatactgac
  8641 ttttcaagtg aaatcatagg atacaaggct attgatggtg gtgtcactcg tgacatagca
  8701 tctacagata cttgttttgc taacaaacat gctgattttg acacatggtt tagccagcgt
  8761 ggtggtagtt atactaatga caaagcttgc ccattgattg ctgcagtcat aacaagagaa
  8821 gtgggttttg tcgtgcctgg tttgcctggc acgatattac gcacaactaa tggtgacttt
  8881 ttgcatttct tacctagagt ttttagtgca gttggtaaca tctgttacac accatcaaaa
  8941 cttatagagt acactgactt tgcaacatca gcttgtgttt tggctgctga atgtacaatt
  9001 tttaaagatg cttctggtaa gccagtacca tattgttatg ataccaatgt actagaaggt
  9061 tctgttgctt atgaaagttt acgccctgac acacgttatg tgctcatgga tggctctatt
  9121 attcaatttc ctaacaccta ccttgaaggt tctgttagag tggtaacaac ttttgattct
  9181 gagtactgta ggcacggcac ttgtgaaaga tcagaagctg gtgtttgtgt atctactagt
  9241 ggtagatggg tacttaacaa tgattattac agatctttac caggagtttt ctgtggtgta
  9301 gatgctgtaa atttacttac taatatgttt acaccactaa ttcaacctat tggtgctttg
  9361 gacatatcag catctatagt agctggtggt attgtagcta tcgtagtaac atgccttgcc
  9421 tactatttta tgaggtttag aagagctttt ggtgaataca gtcatgtagt tgcctttaat
  9481 actttactat tccttatgtc attcactgta ctctgtttaa caccagttta ctcattctta
  9541 cctggtgttt attctgttat ttacttgtac ttgacatttt atcttactaa tgatgtttct
  9601 tttttagcac atattcagtg gatggttatg ttcacacctt tagtaccttt ctggataaca
  9661 attgcttata tcatttgtat ttccacaaag catttctatt ggttctttag taattaccta
  9721 aagagacgtg tagtctttaa tggtgtttcc tttagtactt ttgaagaagc tgcgctgtgc
  9781 acctttttgt taaataaaga aatgtatcta aagttgcgta gtgatgtgct attacctctt
  9841 acgcaatata atagatactt agctctttat aataagtaca agtattttag tggagcaatg
  9901 gatacaacta gctacagaga agctgcttgt tgtcatctcg caaaggctct caatgacttc
  9961 agtaactcag gttctgatgt tctttaccaa ccaccacaaa tctctatcac ctcagctgtt
10021 ttgcagagtg gttttagaaa aatggcattc ccatctggta aagttgaggg ttgtatggta
10081 caagtaactt gtggtacaac tacacttaac ggtctttggc ttgatgacgt agtttactgt
10141 ccaagacatg tgatctgcac ctctgaagac atgcttaacc ctaattatga agatttactc
10201 attcgtaagt ctaatcataa tttcttggta caggctggta atgttcaact cagggttatt
10261 ggacattcta tgcaaaattg tgtacttaag cttaaggttg atacagccaa tcctaagaca
10321 cctaagtata agtttgttcg cattcaacca ggacagactt tttcagtgtt agcttgttac
10381 aatggttcac catctggtgt ttaccaatgt gctatgaggc acaatttcac tattaagggt
10441 tcattcctta atggttcatg tggtagtgtt ggttttaaca tagattatga ctgtgtctct
10501 ttttgttaca tgcaccatat ggaattacca actggagttc atgctggcac agacttagaa
10561 ggtaactttt atggaccttt tgttgacagg caaacagcac aagcagctgg tacggacaca
10621 actattacag ttaatgtttt agcttggttg tacgctgctg ttataaatgg agacaggtgg
10681 tttctcaatc gatttaccac aactcttaat gactttaacc ttgtggctat gaagtacaat
10741 tatgaacctc taacacaaga ccatgttgac atactaggac ctctttctgc tcaaactgga
10801 attgccgttt tagatatgtg tgcttcatta aaagaattac tgcaaaatgg tatgaatgga
10861 cgtaccatat tgggtagtgc tttattagaa gatgaattta caccttttga tgttgttaga
10921 caatgctcag gtgttacttt ccaaagtgca gtgaaaagaa caatcaaggg tacacaccac
10981 tggttgttac tcacaatttt gacttcactt ttagttttag tccagagtac tcaatggtct
11041 ttgttctttt ttttgtatga aaatgccttt ttaccttttg ctatgggtat tattgctatg
11101 tctgcttttg caatgatgtt tgtcaaacat aagcatgcat ttctctgttt gtttttgtta
11161 ccttctcttg ccactgtagc ttattttaat atggtctata tgcctgctag ttgggtgatg
11221 cgtattatga catggttgga tatggttgat actagtttta agctaaaaga ctgtgttatg
11281 tatgcatcag ctgtagtgtt actaatcctt atgacagcaa gaactgtgta tgatgatggt
11341 gctaggagag tgtggacact tatgaatgtc ttgacactcg tttataaagt ttattatggt
11401 aatgctttag atcaagccat ttccatgtgg gctcttataa tctctgttac ttctaactac
11461 tcaggtgtag ttacaactgt catgtttttg gccagaggtg ttgtttttat gtgtgttgag
11521 tattgcccta ttttcttcat aactggtaat acacttcagt gtataatgct agtttattgt
11581 ttcttaggct atttttgtac ttgttacttt ggcctctttt gtttactcaa ccgctacttt
11641 agactgactc ttggtgttta tgattactta gtttctacac aggagtttag atatatgaat
11701 tcacagggac tactcccacc caagaatagc atagatgcct tcaaactcaa cattaaattg
11761 ttgggtgttg gtggcaaacc ttgtatcaaa gtagccactg tacagtctaa aatgtcagat
11821 gtaaagtgca catcagtagt cttactctca gttttgcaac aactcagagt agaatcatca
11881 tctaaattgt gggctcaatg tgtccagtta cacaatgaca ttctcttagc taaagatact
11941 actgaagcct ttgaaaaaat ggtttcacta ctttctgttt tgctttccat gcagggtgct
12001 gtagacataa acaagctttg tgaagaaatg ctggacaaca gggcaacctt acaagctata
12061 gcctcagagt ttagttccct tccatcatat gcagcttttg ctactgctca agaagcttat
12121 gagcaggctg ttgctaatgg tgattctgaa gttgttctta aaaagttgaa gaagtctttg
12181 aatgtggcta aatctgaatt tgaccgtgat gcagccatgc aacgtaagtt ggaaaagatg
12241 gctgatcaag ctatgaccca aatgtataaa caggctagat ctgaggacaa gagggcaaaa
12301 gttactagtg ctatgcagac aatgcttttc actatgctta gaaagttgga taatgatgca
12361 ctcaacaaca ttatcaacaa tgcaagagat ggttgtgttc ccttgaacat aatacctctt
12421 acaacagcag ccaaactaat ggttgtcata ccagactata acacatataa aaatacgtgt
12481 gatggtacaa catttactta tgcatcagca ttgtgggaaa tccaacaggt tgtagatgca
12541 gatagtaaaa ttgttcaact tagtgaaatt agtatggaca attcacctaa tttagcatgg
12601 cctcttattg taacagcttt aagggccaat tctgctgtca aattacagaa taatgagctt
12661 agtcctgttg cactacgaca gatgtcttgt gctgccggta ctacacaaac tgcttgcact
12721 gatgacaatg cgttagctta ctacaacaca acaaagggag gtaggtttgt acttgcactg
12781 ttatccgatt tacaggattt gaaatgggct agattcccta agagtgatgg aactggtact
12841 atctatacag aactggaacc accttgtagg tttgttacag acacacctaa aggtcctaaa
12901 gtgaagtatt tatactttat taaaggatta aacaacctaa atagaggtat ggtacttggt
12961 agtttagctg ccacagtacg tctacaagct ggtaatgcaa cagaagtgcc tgccaattca
13021 actgtattat ctttctgtgc ttttgctgta gatgctgcta aagcttacaa agattatcta
13081 gctagtgggg gacaaccaat cactaattgt gttaagatgt tgtgtacaca cactggtact
13141 ggtcaggcaa taacagtcac accggaagcc aatatggatc aagaatcctt tggtggtgca
13201 tcgtgttgtc tgtactgccg ttgccacata gatcatccaa atcctaaagg attttgtgac
13261 ttaaaaggta agtatgtaca aatacctaca acttgtgcta atgaccctgt gggttttaca
13321 cttaaaaaca cagtctgtac cgtctgcggt atgtggaaag gttatggctg tagttgtgat
13381 caactccgcg aacccatgct tcagtcagct gatgcacaat cgtttttaaa cgggtttgcg
13441 gtgtaagtgc agcccgtctt acaccgtgcg gcacaggcac tagtactgat gtcgtataca
13501 gggcttttga catctacaat gataaagtag ctggttttgc taaattccta aaaactaatt
13561 gttgtcgctt ccaagaaaag gacgaagatg acaatttaat tgattcttac tttgtagtta
13621 agagacacac tttctctaac taccaacatg aagaaacaat ttataattta cttaaggatt
13681 gtccagctgt tgctaaacat gacttcttta agtttagaat agacggtgac atggtaccac
13741 atatatcacg tcaacgtctt actaaataca caatggcaga cctcgtctat gctttaaggc
13801 attttgatga aggtaattgt gacacattaa aagaaatact tgtcacatac aattgttgtg
13861 atgatgatta tttcaataaa aaggactggt atgattttgt agaaaaccca gatatattac
13921 gcgtatacgc caacttaggt gaacgtgtac gccaagcttt gttaaaaaca gtacaattct
13981 gtgatgccat gcgaaatgct ggtattgttg gtgtactgac attagataat caagatctca
14041 atggtaactg gtatgatttc ggtgatttca tacaaaccac gccaggtagt ggagttcctg
14101 ttgtagattc ttattattca ttgttaatgc ctatattaac cttgaccagg gctttaactg
14161 cagagtcaca tgttgacact gacttaacaa agccttacat taagtgggat ttgttaaaat
14221 atgacttcac ggaagagagg ttaaaactct ttgaccgtta ttttaaatat tgggatcaga
14281 cataccaccc aaattgtgtt aactgtttgg atgacagatg cattctgcat tgtgcaaact
14341 ttaatgtttt attctctaca gtgttcccac ttacaagttt tggaccacta gtgagaaaaa
14401 tatttgttga tggtgttcca tttgtagttt caactggata ccacttcaga gagctaggtg
14461 ttgtacataa tcaggatgta aacttacata gctctagact tagttttaag gaattacttg
14521 tgtatgctgc tgaccctgct atgcacgctg cttctggtaa tctattacta gataaacgca
14581 ctacgtgctt ttcagtagct gcacttacta acaatgttgc ttttcaaact gtcaaacccg
14641 gtaattttaa caaagacttc tatgactttg ctgtgtctaa gggtttcttt aaggaaggaa
14701 gttctgttga attaaaacac ttcttctttg ctcaggatgg taatgctgct atcagcgatt
14761 atgactacta tcgttataat ctaccaacaa tgtgtgatat cagacaacta ctatttgtag
14821 ttgaagttgt tgataagtac tttgattgtt acgatggtgg ctgtattaat gctaaccaag
14881 tcatcgtcaa caacctagac aaatcagctg gttttccatt taataaatgg ggtaaggcta
14941 gactttatta tgattcaatg agttatgagg atcaagatgc acttttcgca tatacaaaac
15001 gtaatgtcat ccctactata actcaaatga atcttaagta tgccattagt gcaaagaata
15061 gagctcgcac cgtagctggt gtctctatct gtagtactat gaccaataga cagtttcatc
15121 aaaaattatt gaaatcaata gccgccacta gaggagctac tgtagtaatt ggaacaagca
15181 aattctatgg tggttggcac aatatgttaa aaactgttta tagtgatgta gaaaaccctc
15241 accttatggg ttgggattat cctaaatgtg atagagccat gcctaacatg cttagaatta
15301 tggcctcact tgttcttgct cgcaaacata caacgtgttg tagcttgtca caccgtttct
15361 atagattagc taatgagtgt gctcaagtat tgagtgaaat ggtcatgtgt ggcggttcac
15421 tatatgttaa accaggtgga acctcatcag gagatgccac aactgcttat gctaatagtg
15481 tttttaacat ttgtcaagct gtcacggcca atgttaatgc acttttatct actgatggta
15541 acaaaattgc cgataagtat gtccgcaatt tacaacacag actttatgag tgtctctata
15601 gaaatagaga tgttgacaca gactttgtga atgagtttta cgcatatttg cgtaaacatt
15661 tctcaatgat gatactctct gacgatgctg ttgtgtgttt caatagcact tatgcatctc
15721 aaggtctagt ggctagcata aagaacttta agtcagttct ttattatcaa aacaatgttt
15781 ttatgtctga agcaaaatgt tggactgaga ctgaccttac taaaggacct catgaatttt
15841 gctctcaaca tacaatgcta gttaaacagg gtgatgatta tgtgtacctt ccttacccag
15901 atccatcaag aatcctaggg gccggctgtt ttgtagatga tatcgtaaaa acagatggta
15961 cacttatgat tgaacggttc gtgtctttag ctatagatgc ttacccactt actaaacatc
16021 ctaatcagga gtatgctgat gtctttcatt tgtacttaca atacataaga aagctacatg
16081 atgagttaac aggacacatg ttagacatgt attctgttat gcttactaat gataacactt
16141 caaggtattg ggaacctgag ttttatgagg ctatgtacac accgcataca gtcttacagg
16201 ctgttggggc ttgtgttctt tgcaattcac agacttcatt aagatgtggt gcttgcatac
16261 gtagaccatt cttatgttgt aaatgctgtt acgaccatgt catatcaaca tcacataaat
16321 tagtcttgtc tgttaatccg tatgtttgca atgctccagg ttgtgatgtc acagatgtga
16381 ctcaacttta cttaggaggt atgagctatt attgtaaatc acataaacca cccattagtt
16441 ttccattgtg tgctaatgga caagtttttg gtttatataa aaatacatgt gttggtagcg
16501 ataatgttac tgactttaat gcaattgcaa catgtgactg gacaaatgct ggtgattaca
16561 ttttagctaa cacctgtact gaaagactca agctttttgc agcagaaacg ctcaaagcta
16621 ctgaggagac atttaaactg tcttatggta ttgctactgt acgtgaagtg ctgtctgaca
16681 gagaattaca tctttcatgg gaagttggta aacctagacc accacttaac cgaaattatg
16741 tctttactgg ttatcgtgta actaaaaaca gtaaagtaca aataggagag tacacctttg
16801 aaaaaggtga ctatggtgat gctgttgttt accgaggtac aacaacttac aaattaaatg
16861 ttggtgatta ttttgtgctg acatcacata cagtaatgcc attaagtgca cctacactag
16921 tgccacaaga gcactatgtt agaattactg gcttataccc aacactcaat atctcagatg
16981 agttttctag caatgttgca aattatcaaa aggttggtat gcaaaagtat tctacactcc
17041 agggaccacc tggtactggt aagagtcatt ttgctattgg cctagctctc tactaccctt
17101 ctgctcgcat agtgtataca gcttgctctc atgccgctgt tgatgcacta tgtgagaagg
17161 cattaaaata tttgcctata gataaatgta gtagaattat acctgcacgt gctcgtgtag
17221 agtgttttga taaattcaaa gtgaattcaa cattagaaca gtatgtcttt tgtactgtaa
17281 atgcattgcc tgagacgaca gcagatatag ttgtctttga tgaaatttca atggccacaa
17341 attatgattt gagtgttgtc aatgccagat tacgtgctaa gcactatgtg tacattggcg
17401 accctgctca attacctgca ccacgcacat tgctaactaa gggcacacta gaaccagaat
17461 atttcaattc agtgtgtaga cttatgaaaa ctataggtcc agacatgttc ctcggaactt
17521 gtcggcgttg tcctgctgaa attgttgaca ctgtgagtgc tttggtttat gataataagc
17581 ttaaagcaca taaagacaaa tcagctcaat gctttaaaat gttttataag ggtgttatca
17641 cgcatgatgt ttcatctgca attaacaggc cacaaatagg cgtggtaaga gaattcctta
17701 cacgtaaccc tgcttggaga aaagctgtct ttatttcacc ttataattca cagaatgctg
17761 tagcctcaaa gattttggga ctaccaactc aaactgttga ttcatcacag ggctcagaat
17821 atgactatgt catattcact caaaccactg aaacagctca ctcttgtaat gtaaacagat
17881 ttaatgttgc tattaccaga gcaaaagtag gcatactttg cataatgtct gatagagacc
17941 tttatgacaa gttgcaattt acaagtcttg aaattccacg taggaatgtg gcaactttac
18001 aagctgaaaa tgtaacagga ctctttaaag attgtagtaa ggtaatcact gggttacatc
18061 ctacacaggc acctacacac ctcagtgttg acactaaatt caaaactgaa ggtttatgtg
18121 ttgacgtacc tggcatacct aaggacatga cctatagaag actcatctct atgatgggtt
18181 ttaaaatgaa ttatcaagtt aatggttacc ctaacatgtt tatcacccgc gaagaagcta
18241 taagacatgt acgtgcatgg attggcttcg atgtcgaggg gtgtcatgct actagagaag
18301 ctgttggtac caatttacct ttacagctag gtttttctac aggtgttaac ctagttgctg
18361 tacctacagg ttatgttgat acacctaata atacagattt ttccagagtt agtgctaaac
18421 caccgcctgg agatcaattt aaacacctca taccacttat gtacaaagga cttccttgga
18481 atgtagtgcg tataaagatt gtacaaatgt taagtgacac acttaaaaat ctctctgaca
18541 gagtcgtatt tgtcttatgg gcacatggct ttgagttgac atctatgaag tattttgtga
18601 aaataggacc tgagcgcacc tgttgtctat gtgatagacg tgccacatgc ttttccactg
18661 cttcagacac ttatgcctgt tggcatcatt ctattggatt tgattacgtc tataatccgt
18721 ttatgattga tgttcaacaa tggggtttta caggtaacct acaaagcaac catgatctgt
18781 attgtcaagt ccatggtaat gcacatgtag ctagttgtga tgcaatcatg actaggtgtc
18841 tagctgtcca cgagtgcttt gttaagcgtg ttgactggac tattgaatat cctataattg
18901 gtgatgaact gaagattaat gcggcttgta gaaaggttca acacatggtt gttaaagctg
18961 cattattagc agacaaattc ccagttcttc acgacattgg taaccctaaa gctattaagt
19021 gtgtacctca agctgatgta gaatggaagt tctatgatgc acagccttgt agtgacaaag
19081 cttataaaat agaagaatta ttctattctt atgccacaca ttctgacaaa ttcacagatg
19141 gtgtatgcct attttggaat tgcaatgtcg atagatatcc tgctaattcc attgtttgta
19201 gatttgacac tagagtgcta tctaacctta acttgcctgg ttgtgatggt ggcagtttgt
19261 atgtaaataa acatgcattc cacacaccag cttttgataa aagtgctttt gttaatttaa
19321 aacaattacc atttttctat tactctgaca gtccatgtga gtctcatgga aaacaagtag
19381 tgtcagatat agattatgta ccactaaagt ctgctacgtg tataacacgt tgcaatttag
19441 gtggtgctgt ctgtagacat catgctaatg agtacagatt gtatctcgat gcttataaca
19501 tgatgatctc agctggcttt agcttgtggg tttacaaaca atttgatact tataacctct
19561 ggaacacttt tacaagactt cagagtttag aaaatgtggc ttttaatgtt gtaaataagg
19621 gacactttga tggacaacag ggtgaagtac cagtttctat cattaataac actgtttaca
19681 caaaagttga tggtgttgat gtagaattgt ttgaaaataa aacaacatta cctgttaatg
19741 tagcatttga gctttgggct aagcgcaaca ttaaaccagt accagaggtg aaaatactca
19801 ataatttggg tgtggacatt gctgctaata ctgtgatctg ggactacaaa agagatgctc
19861 cagcacatat atctactatt ggtgtttgtt ctatgactga catagccaag aaaccaactg
19921 aaacgatttg tgcaccactc actgtctttt ttgatggtag agttgatggt caagtagact
19981 tatttagaaa tgcccgtaat ggtgttctta ttacagaagg tagtgttaaa ggtttacaac
20041 catctgtagg tcccaaacaa gctagtctta atggagtcac attaattgga gaagccgtaa
20101 aaacacagtt caattattat aagaaagttg atggtgttgt ccaacaatta cctgaaactt
20161 actttactca gagtagaaat ttacaagaat ttaaacccag gagtcaaatg gaaattgatt
20221 tcttagaatt agctatggat gaattcattg aacggtataa attagaaggc tatgccttcg
20281 aacatatcgt ttatggagat tttagtcata gtcagttagg tggtttacat ctactgattg
20341 gactagctaa acgttttaag gaatcacctt ttgaattaga agattttatt cctatggaca
20401 gtacagttaa aaactatttc ataacagatg cgcaaacagg ttcatctaag tgtgtgtgtt
20461 ctgttattga tttattactt gatgattttg ttgaaataat aaaatcccaa gatttatctg
20521 tagtttctaa ggttgtcaaa gtgactattg actatacaga aatttcattt atgctttggt
20581 gtaaagatgg ccatgtagaa acattttacc caaaattaca atctagtcaa gcgtggcaac
20641 cgggtgttgc tatgcctaat ctttacaaaa tgcaaagaat gctattagaa aagtgtgacc
20701 ttcaaaatta tggtgatagt gcaacattac ctaaaggcat aatgatgaat gtcgcaaaat
20761 atactcaact gtgtcaatat ttaaacacat taacattagc tgtaccctat aatatgagag
20821 ttatacattt tggtgctggt tctgataaag gagttgcacc aggtacagct gttttaagac
20881 agtggttgcc tacgggtacg ctgcttgtcg attcagatct taatgacttt gtctctgatg
20941 cagattcaac tttgattggt gattgtgcaa ctgtacatac agctaataaa tgggatctca
21001 ttattagtga tatgtacgac cctaagacta aaaatgttac aaaagaaaat gactctaaag
21061 agggtttttt cacttacatt tgtgggttta tacaacaaaa gctagctctt ggaggttccg
21121 tggctataaa gataacagaa cattcttgga atgctgatct ttataagctc atgggacact
21181 tcgcatggtg gacagccttt gttactaatg tgaatgcgtc atcatctgaa gcatttttaa
21241 ttggatgtaa ttatcttggc aaaccacgcg aacaaataga tggttatgtc atgcatgcaa
21301 attacatatt ttggaggaat acaaatccaa ttcagttgtc ttcctattct ttatttgaca
21361 tgagtaaatt tccccttaaa ttaaggggta ctgctgttat gtctttaaaa gaaggtcaaa
21421 tcaatgatat gattttatct cttcttagta aaggtagact tataattaga gaaaacaaca
21481 gagttgttat ttctagtgat gttcttgtta acaactaaac gaacaatgtt tgtttttctt
21541 gttttattgc cactagtctc tagtcagtgt gttaatctta caaccagaac tcaattaccc
21601 cctgcataca ctaattcttt cacacgtggt gtttattacc ctgacaaagt tttcagatcc
21661 tcagttttac attcaactca ggacttgttc ttacctttct tttccaatgt tacttggttc
21721 catgttatct ctgggaccaa tggtactaag aggtttgata accctgtect accatttaat
21781 gatggtgttt attttgcttc cattgagaag tctaacataa taagaggctg gatttttggt
21841 actactttag attcgaagac ccagtcccta cttattgtta ataacgctac taatgttgtt
21901 attaaagtct gtgaatttca attttgtaat gatccatttt tggaccacaa aaacaacaaa
21961 agttggatgg aaagtgagtt cagagtttat tctagtgcga ataattgcac ttttgaatat
22021 gtctctcagc cttttcttat ggaccttgaa ggaaaacagg gtaatttcaa aaatcttagg
22081 gaatttgtgt ttaagaatat tgatggttat tttaaaatat attctaagca cacgcctatt
22141 atagtgcgtg agccagaaga tctccctcag ggtttttcgg ctttagaacc attggtagat
22201 ttgccaatag gtattaacat cactaggttt caaactttac ttgctttaca tagaagttat
22261 ttgactcctg gtgattcttc ttcaggttgg acagctggtg ctgcagctta ttatgtgggt
22321 tatcttcaac ctaggacttt tctattaaaa tataatgaaa atggaaccat tacagatgct
22381 gtagactgtg cacttgaccc tctctcagaa acaaagtgta cgttgaaatc cttcactgta
22441 gaaaaaggaa tctatcaaac ttctaacttt agagtccaac caacagaatc tattgttaga
22501 tttcctaata ttacaaactt gtgccctttt gatgaagttt ttaacgccac cagatttgca
22561 tctgtttatg cttggaacag gaagagaatc agcaactgtg ttgctgatta ttctgtccta
22621 tataatctcg caccattttt cacttttaag tgttatggag tgtctcctac taaattaaat
22681 gatctctgct ttactaatgt ctatgcagat tcatttgtaa ttagaggtga tgaagtcaga
22741 caaatcgctc cagggcaaac tggaaatatt gctgattata attataaatt accagatgat
22801 tttacaggct gcgttatagc ttggaattct aacaagcttg attctaaggt tagtggtaat
22861 tataattacc tgtatagatt gtttaggaag tctaatctca aaccttttga gagagatatt
22921 tcaactgaaa tctatcaggc cggtaacaaa ccttgtaatg gtgttgcagg ttttaattgt
22981 tactttcctt tacgatcata tagtttccga cccacttatg gtgttggtta ccaaccatac
23041 agagtagtag tactttcttt tgaacttcta catgcaccag caactgtttg tggacctaaa
23101 aagtctacta atttggttaa aaacaaatgt gtcaatttca acttcaatgg tttaaaaggc
23161 acaggtgttc ttactgagtc taacaaaaag tttctgcctt tccaacaatt tggcagagac
23221 attgctgaca ctactgatgc tgtccgtgat ccacagacac ttgagattct tgacattaca
23281 ccatgttctt ttggtggtgt cagtgttata acaccaggaa caaatacttc taaccaggtt
23341 gctgttcttt atcagggtgt taactgcaca gaagtccctg ttgctattca tgcagatcaa
23401 cttactccta cttggcgtgt ttattctaca ggttctaatg tttttcaaac acgtgcaggc
23461 tgtttaatag gggctgaata tgtcaacaac tcatatgagt gtgacatacc cattggtgca
23521 ggtatatgcg ctagttatca gactcagact aagtctcatc ggcgggcacg tagtgtagct
23581 agtcaatcca tcattgccta cactatgtca cttggtgcag aaaattcagt tgcttactct
23641 aataactcta ttgccatacc cacaaatttt actattagtg ttaccacaga aattctacca
23701 gtgtctatga ccaagacatc agtagattgt acaatgtaca tttgtggtga ttcaactgaa
23761 tgcagcaatc ttttgttgca atatggcagt ttttgtacac aattaaaacg tgctttaact
23821 ggaatagctg ttgaacaaga caaaaacacc caagaagttt ttgcacaagt caaacaaatt
23881 tacaaaacac caccaattaa atattttggt ggttttaatt tttcacaaat attaccagat
23941 ccatcaaaac caagcaagag gtcatttatt gaagatctac ttttcaacaa agtgacactt
24001 gcagatgctg gcttcatcaa acaatatggt gattgccttg gtgatattgc tgctagagac
24061 ctcatttgtg cacaaaagtt taaaggcctt actgttttgc cacctttgct cacagatgaa
24121 atgattgctc aatacacttc tgcactgtta gcgggtacaa tcacttctgg ttggaccttt
24181 ggtgcaggtg ctgcattaca aataccattt gctatgcaaa tggcttatag gtttaatggt
24241 attggagtta cacagaatgt tctctatgag aaccaaaaat tgattgccaa ccaatttaat
24301 agtgctattg gcaaaattca agactcactt tcttccacag caagtgcact tggaaaactt
24361 caagatgtgg tcaaccataa tgcacaagct ttaaacacgc ttgttaaaca acttagctcc
24421 aaatttggtg caatttcaag tgttttaaat gatatctttt cacgtcttga caaagttgag
24481 gctgaagtgc aaattgatag gttgatcaca ggcagacttc aaagtttgca gacatatgtg
24541 actcaacaat taattagagc tgcagaaatc agagcttctg ctaatcttgc tgctactaaa
24601 atgtcagagt gtgtacttgg acaatcaaaa agagttgatt tttgtggaaa gggctatcat
24661 cttatgtcct tccctcagtc agcacctcat ggtgtagtct tcttgcatgt gacttatgtc
24721 cctgcacaag aaaagaactt cacaactgct cctgccattt gtcatgatgg aaaagcacac
24781 tttcctcgtg aaggtgtctt tgtttcaaat ggcacacact ggtttgtaac acaaaggaat
24841 ttttatgaac cacaaatcat tactacagac aacacatttg tgtctggtaa ctgtgatgtt
24901 gtaataggaa ttgtcaacaa cacagtttat gatcctttgc aacctgaatt agattcattc
24961 aaggaggagt tagataaata ttttaagaat catacatcac cagatgttga tttaggtgac
25021 atctctggca ttaatgcttc agttgtaaac attcaaaaag aaattgaccg cctcaatgag
25081 gttgccaaga atttaaatga atctctcatc gatctccaag aacttggaaa gtatgagcag
25141 tatataaaat ggccatggta catttggcta ggttttatag ctggcttgat tgccatagta
25201 atggtgacaa ttatgctttg ctgtatgacc agttgctgta gttgtctcaa gggctgttgt
25261 tcttgtggat cctgctgcaa atttgatgaa gacgactctg agccagtgct caaaggagtc
25321 aaattacatt acacataaac gaacttatgg atttgtttat gagaatcttc acaattggaa
25381 ctgtaacttt gaagcaaggt gaaatcaagg atgctactcc ttcagatttt gttcgcgcta
25441 ctgcaacgat accgatacaa gcctcactcc ctttcggatg gcttattgtt ggcgttgcac
25501 ttcttgctgt ttttcagagc gcttccaaaa tcataactct caaaaagaga tggcaactag
25561 cactctccaa gggtgttcac tttgtttgca acttgctgtt gttgtttgta acagtttact
25621 cacacctttt gctcgttgct gctggccttg aagccccttt tctctatctt tatgctttag
25681 tctacttctt gcagagtata aactttgtaa gaataataat gaggctttgg ctttgctgga
25741 aatgccgttc caaaaaccca ttactttatg atgccaacta ttttctttgc tggcatacta
25801 attgttacga ctattgtata ccttacaata gtgtaacttc ttcaattgtc attacttcag
25861 gtgatggcac aacaagtcct atttctgaac atgactacca gattggtggt tatactgaaa
25921 aatgggaatc tggagtaaaa gactgtgttg tattacacag ttacttcact tcagactatt
25981 accagctgta ctcaactcaa ttgagtacag acactggtgt tgaacatgtt accttcttca
26041 tctacaataa aattgttgat gagcctgaag aacatgtcca aattcacaca atcgacggtt
26101 catccggagt tgttaatcca gtaatggaac caatttatga tgaaccgacg acgactacta
26161 gcgtgccttt gtaagcacaa gctgatgagt acgaacttat gtactcattc gtttcggaag
26221 agataggtac gttaatagtt aatagcgtac ttctttttct tgctttcgtg gtattcttgc
26281 tagttacact agccatcctt actgcgcttc gattgtgtgc gtactgctgc aatattgtta
26341 acgtgagtct tgtaaaacct tctttttacg tttactctcg tgttaaaaat ctgaattctt
26401 ctagagttcc tgatcttctg gtctaaacga actaaatatt atattagttt ttctgtttgg
26461 aactttaatt ttagccatgg caggttccaa cggtactatt accgttgaag agcttaaaaa
26521 gctccttgaa gaatggaacc tagtaatagg tttcctattc cttacatgga tttgtcttct
26581 acaatttgcc tatgccaaca ggaataggtt tttgtatata attaagttaa ttttcctctg
26641 gctgttatgg ccagtaactt taacttgttt tgtgcttgct gctgtttaca gaataaattg
26701 gatcaccggt ggaattgcta tcgcaatggc ttgtcttgta ggcttgatgt ggctcagcta
26761 cttcattgct tctttcagac tgtttgcgcg tacgcgttcc atgtggtcat tcaatccaga
26821 aactaacatt cttctcaacg tgccactcca tggcactatt ctgaccagac cgcttctaga
26881 aagtgaactc gtaatcggag ctgtgatcct tcgtggacat cttcgtattg ctggacacca
26941 tctaggacgc tgtgacatca aggacctgcc taaagaaatc actgttgcta catcacgaac
27001 gctttcttat tacaaattgg gagcttcgca gcgtgtagca ggtgactcag gttttgctgc
27061 atacagtcgc tacaggattg gcaactataa attaaacaca gaccattcca gtagcagtga
27121 caatattgct ttgcttgtac agtaagtgac aacagatgtt tcatctcgtt gactttcagg
27181 ttactatagc agagatatta ctaattatta tgcggacttt taaagtttcc atttggaatc
27241 ttgattacat cataaacctc ataattaaaa atttatctaa gtcactaact gagaataaat
27301 attctcaatt agatgaagag caaccaatgg agattgatta aacgaacatg aaaattattc
27361 ttttcttggc actgataaca ctcgctactt gtgagcttta tcactaccaa gagtgtgtta
27421 gaggtacaac agtactttta aaagaacctt gctcttctgg aacatacgag ggcaattcac
27481 catttcatcc tctagctgat aacaaatttg cactgacttg ctttagcact caatttgctt
27541 ttgcttgtcc tgacggcgta aaacacgtct atcagttacg tgccagatca gtttcaccta
27601 aactgttcat cagacaagag gaagttcaag aactttactc tccaattttt cttattgttg
27661 cggcaatagt gtttataaca ctttgcttca cactcaaaag aaagacagaa tgattgaact
27721 ttcattaatt gacttctatt tgtgcttttt agcctttctg ttattccttg ttttaattat
27781 gcttattatc ttttggttct cacttgaact gcaagatcat aatgaaactt gtcacgccta
27841 aacgaacatg aaatttcttg ttttcttagg aatcatcaca actgtagctg catttcacca
27901 agaatgtagt ttacagtcat gtactcaaca tcaaccatat gtagttgatg acccgtgtcc
27961 tattcacttc tattctaaat ggtatattag agtaggagct agaaaatcag cacctttaat
28021 tgaattgtgc gtggatgagg ctggttctaa atcacccatt cagtacatcg atatcggtaa
28081 ttatacagtt tcctgtttac cttttacaat taattgccag gaacctaaat tgggtagtct
28141 tgtagtgcgt tgttcgttct atgaagactt tttagagtat catgacgttc gtgttgtttt
28201 agatttcatc taaacgaaca aacttaaatg tctgataatg gaccccaaaa tcagcgaaat
28261 gcactccgca ttacgtttgg tggaccctca gattcaactg gcagtaacca gaatggtggg
28321 gcgcgatcaa aacaacgtcg gccccaaggt ttacccaata atactgcgtc ttggttcacc
28381 gctctcactc aacatggcaa ggaagacctt aaattccctc gaggacaagg cgttccaatt
28441 aacaccaata gcagtccaga tgaccaaatt ggctactacc gaagagctac cagacgaatt
28501 cgtggtggtg acggtaaaat gaaagatctc agtccaagat ggtatttcta ctacctagga
28561 actgggccag aagctggact tccctatggt gctaacaaag acggcatcat atgggttgca
28621 actgagggag ccttgaatac accaaaagat cacattggca cccgcaatcc tgctaacaat
28681 gctgcaatcg tgctacaact tcctcaagga acaacattgc caaaaggctt ctacgcagaa
28741 gggagcagag gcggcagtca agcctcttct cgttcctcat cacgtagtcg caacagttca
28801 agaaattcaa ctccaggcag cagtaaacga acttctcctg ctagaatggc tggcaatggc
28861 ggtgatgctg ctcttgcttt gctgctgctt gacagattga accagcttga gagcaaaatg
28921 tctggtaaag gccaacaaca acaaggccaa actgtcacta agaaatctgc tgctgaggct
28981 tctaagaagc ctcggcaaaa acgtactgcc actaaagcat acaatgtaac acaagctttc
29041 ggcagacgtg gtccagaaca aacccaagga aattttgggg accaggaact aatcagacaa
29101 ggaactgatt acaaacattg gccgcaaatt gcacaatttg cccccagcgc ttcagcgttc
29161 ttcggaatgt cgcgcattgg catggaagtc acaccttcgg gaacgtggtt gacctacaca
29221 ggtgccatca aattggatga caaagatcca aatttcaaag atcaagtcat tttgctgaat
29281 aagcatattg acgcatacaa aacattccca ccaacagagc ctaaaaagga caaaaagaag
29341 aaggctgatg aaactcaagc cttaccgcag agacagaaga aacagcaaac tgtgactctt
29401 cttcctgctg cagatttgga tgatttctcc aaacaattgc aacaatccat gagcagtgct
29461 gactcaactc aggcctaaac tcatgcagac cacacaaggc agatgggcta tataaacgtt
29521 ttcgcttttc cgtttacgat atatagtcta ctcttgtgca gaatgaattc tcgtaactac
29581 atagcacaag tagatgtagt taactttaat ctcacatagc aatctttaat cagtgtgtaa
29641 cattagggag gacttgaaag agccaccaca ttttcaccga ggccacgcgg agtacgatcg
29701 agtgtacagt gaacaatgct agggagagct gcctatatgg aagagcccta atgtgtaaaa
29761 ttaattttag tagtgctatc cccatgtgat tttaatagct tctt
```

The SARS-CoV-2 is a p coronavirus belonging to the Coronaviridae family known to cause COVID-19. It consists of ORFs that code for structural, non-structural, and accessory proteins. The S (spike protein), N (nucleocapsid protein), M (membrane protein), E (envelope protein) form the structural proteins that play a vital role in the assembly of the viral particles. The S protein is shaped like a clove with two subunits S1 and S2 which promotes receptor binding and membrane fusion respectively. The N protein consists of an NTD, serine-rich linker and CTD. It enhances viral entry and performs post-fusion cellular processes necessary for viral survival in the host. The E protein promotes virion formation and viral pathogenicity while M protein forms ribonucleoproteins and mediates inflammatory responses in hosts (Satarker and Nampoothiri. Arch Med Res. 2020 August; 51(6): 482-491). The methods provided herein can elucidate the function and effect of variation/mutation(s) in each of the structural, non-structural and accessory proteins.

### Cloning/Assembly of Viral Fragments

The invention is a method to rapidly clone viral genomes, such as SARS-CoV-2 and variants thereof, without the need for laborious cloning strategies that can limit accessibility.

In one embodiment, the invention is carried out by cloning of the viral genome into different segments flanked by suitable restriction enzyme sites. The viral genome at be divided into a plurality of segments, such as 2, 3, 4, 5, 6, 7, 8, 9, 10, 11, 12, 13, 14, 15, 16, 17, 18, 19, 20 or more fragments/segments, allowing for different permutations of the segments to be made. The segments can be divided so as to comprise or contain one or more viral open reading frame(s) or the segments can be of a certain length of the viral DNA. Segments can also be designed so to a have one or more mutations/additions/deletions added to the sequence of the segment (to investigate the effect that mutation has on the virus, such as viral on replication, infectivity etc.). The mutations can be in an open reading frame, such as a mutation to the spike protein nucleic acid or protein sequence. Adapters can be added to the 5′ and 3′ ends of each segment, wherein the adapters comprise the recognition site for a Type IIS restriction endonuclease, such as BsaI, resulting DNA sections that are flanked by Type IIS restriction endonuclease sites with opposite orientations; alternatively, the cloning plasmid can comprise Type IIS restriction endonuclease recognition sites. To aid in annealing and ligation of the plurality of segments in the correct order and orientation, each segment is a series of overlapping segments in which segment has a defined length of overlap, said overlap comprising unique, non-palindromic DNA sequences. The DNA segments can be derived by PCR using primer sequences to create the overlapping sequence between the sequences to be joined (or the DNA segments can be created synthetically by methods known to the art). If naturally occurring Type IIS restriction endonuclease sites occur in the genome of the virus, such Type IIS restriction endonuclease sites can be removed by methods know to an art worker, such as by PCR mutagenesis.

Type IIS restriction endonucleases are restriction endonucleases of which the restriction site to one side lies outside its asymmetric non-palindromic recognition sequence. Type ITS restriction endonucleases are known to a person skilled in the art. Examples of type IIs restriction endonucleases include BbsI, BbvI, BcoDI, BfbAI, BsaI, BsnAI, BsnFI, BspMI, BtgZI, Esp3I, FokI, PaqCI, SfaNI, BaeI, and HgaI.

Each segment can be then individually cloned in separate cloning plasmids, wherein each cloning plasmid can comprise a cloning site that is flanked on both sides by Type IIS restriction endonuclease recognition sites, said sites positioned to allow removal by digestion with the class IIS enzyme or enzymes of a defined number of bases from one strand on both ends of the fragment. The plasmids can be placed in a host cell, such as a bacterial cell (e.g., E. coli), where the plasmid can be reproduced/increase in copy number.

The plasmid insert comprising the viral DNA segment can be validated, such as by sequencing or mapping, such as restriction mapping. The clones can then be digested with, for example a Type IIS restriction endonuclease, thereby releasing the insert viral DNA segment (now optionally modified by the removal of the defined number of bases from one strand at each terminus). Such insert segments can be annealed and ligated together and cloned into a destination vector, such as a BAC, so as to create a viral genome with the desired segments, in the desired order and the desired orientation.

For example, in one embodiment, the insert segments are mixed and incubated with a suitable destination plasmid (e.g., pBAC, YAC or any vector that can handle a large genome; the vector can include one or more of the following: a promoter such as CMV, EF1a, RSV, hPGK, SFFV etc.; a T7 or SP6 promoter; HDVrz, hammerhead ribozyme or hairpin ribozyme; SV40 polyA, hGH, BGH or rbGlob polyA sequences) in a Golden Gate assembly reaction to generate a viral genome construct, such as the full-length SARS-CoV-2 genome clone or a variant thereof. The insert in this plasmid can be sequence verified and utilized to produce, for example, SARS-CoV-2 full-length genomic RNA by in vitro transcription or the vector can be electroporated into cells to generate, for example, SARS-CoV-2 virus and variants thereof.

In one embodiment, the viral genome clone is full-length SARS-CoV-2. In one embodiment, one or more segments are not included in the viral genome clone, such as a segment coding for viral spike protein or other open reading frame. In another embodiment, the segments are not all from the same virus, for example, two or more sections of Delta, Omicron, SARS-CoV-2 or a combination thereof are cloned in the vector, such pBAC (such as substituting the Omicron spike protein with Delta's or another variant or mutant). In another embodiment, the segments contain either naturally occurring variants or engineered mutations (so as to determine the effect of those mutations).

In embodiment, to enable the rapid cloning strategy, the SARS-CoV-2 genome, for example, is divided into 10 fragments (the viral genome can be dived into greater or fewer fragments if the genome as greater or fewer coding regions) that correspond to different coding regions of the genome and are as follows:

TABLE 1
Characteristics of SARS-CoV-2 genome fragments.

| | Overhang | | | | |
|---|---|---|---|---|---|
| | 5′ | 3′ | nt | nt | ORF |
| F1 | ATTA | GTGC | 1 | 2721 | ORF1a (nsp1&2) |
| F2 | GTGC | GAGA | 2718 | 5454 | ORF1a (nsp3) |
| F3 | GAGA | GTAA | 5451 | 8556 | ORF1a (nsp3) |
| F4 | GTAA | TCTA | 8553 | 11846 | ORF1a (nap4-6) |
| F5 | TCTA | TGCA | 11843 | 15090 | ORF1a (nsp7-11), ORF1ab (nsp12) |
| F6 | TGCA | GCTG | 15087 | 18043 | ORF1ab (nsp12&13) |
| F7 | GCTG | CAAT | 18040 | 21564 | ORF1ab (nsp14-16) |
| F8 | CAAT | GAAC | 21561 | 25390 | S |
| F9 | GAAC | ACGA | 25387 | 27891 | ORF3a/b, E, M, ORF6, ORF7a/b |
| F10 | ACGA | AAAA | 27888 | 29908 | ORF8, N, ORF9b/c, ORF10 |

These fragments can either be PCR amplified from SARS-CoV-2 viral cDNA or can be synthesized from many available commercial sources/techniques. To enable clonal verification of these fragments and to prepare mutants as necessary, the fragments are cloned into pUC19 based vector/plasmids with the bidirectional tonB terminator upstream and the T7Te and rrnB T1 terminators downstream of the SARS-CoV-2 sequence.

To enable assembly of the full-length SARS-CoV-2 genome using BsaI-mediated Golden Gate assembly, the two BsaI sites in the genome (WA1 nt 17966 and nt 24096) are eliminated by introducing the following synonymous mutations (WA1 nt C17976T and nt C24106T) in fragments F6 and F8, respectively.

The pBAC (bacterial artificial chromosome) vector that can handle the full-length genome was purchased from Lucigen (cat #42032-1). This vector was modified to include a CMV promoter, T7 promoter, BsaI sites, an HDVrz and SV40 polyA. The BsaI site at nt 2302 was mutated (C2307T) to allow use in the BsaI-mediated Golden Gate assembly.

A schematic of the method is shown in FIG. 1A.

For the Golden Gate assembly, the ten fragments as well as the pBAC vector are mixed in stoichiometric ratio and in 1× T4 DNA ligase buffer. To the mixture is then added BsaI and T4 DNA ligase and the reaction can be cycled as follows: Cycle 30 times: 37° C. for 5 min and 16° C. for 5 min, followed by 37° C. for 5 min, 60 C for 5 min and 12° C. for infinity (until needed/used).

### Generation of Infectious Clones

Assembled vector can electroporated into cells, such as EPI300 cells, and plated onto LB+chloramphenicol plates, and grown at 37 C for 24 hr. Generally, only the small colonies are picked as those containing the full-length genome while large colonies typically are background from undigested vector. The colonies can be cultured in LB30 media+12.5 ug/mL chloramphenicol for 12 hours at 37° C. and induced, for example, with arabinose to yield high copy number for 12 hours at 37° C.

The vector, such as the pBAC SARS-CoV-2 vector, can then be transfected directly into, for example, BHK21 cells (FIG. 2A) and then the resulting virus passaged onto cells for propagation (e.g., Vero TMPRSS2 cells). If desired, RNA can be prepared using in vitro transcription and subsequently electroporated into, for example, BHK21 cells (FIG. 2A) to produce virus.

### EXAMPLES

The following examples are intended to further illustrate certain embodiments of the invention and is not intended to limit the scope of the invention in any way.

### Example I

### Introduction

Severe acute respiratory syndrome coronavirus 2 (SARS-CoV-2) is the causative agent of the coronavirus disease 2019 (COVID-19) pandemic. The pandemic continues as a major public health issue worldwide. As of October 2022, more than 600 million people have been infected with it and more than 6.5 million have died¹. The continuous emergence of viral variants represents a major threat to our pandemic countermeasures due to enhanced transmission²⁻⁴ and antibody neutralization escape⁵.

The emergence of the Omicron variant in November 2021 was especially concerning due to the large number of mutations throughout the genome (53 nonsynonymous mutations) and 34 mutations in the Spike protein alone. While Omicron infections spread significantly more rapidly than previous variants, they are associated with fewer symptoms and lower hospitalization rates⁶⁻⁸. Accordingly, the Omicron variant is attenuated in cell culture⁹⁻¹² and animal models of infection¹³⁻¹⁵. An evolutionary tradeoff appears to exist between increased viral spread and diminished infection severity in the context of an increasingly immunized human population. This tradeoff may have arisen only recently as adaptive evolution of SARS-CoV-2 prior to the emergence of Omicron was mainly characterized by purifying selection¹⁶.

SARS-CoV-2 is an enveloped positive-strand RNA virus in the family Coronaviridae in the order Nidovirales¹⁷. Its 30 kb genome contains at least 14 known open reading frames (FIG. 1A). The 5′ two-thirds of the genome encompass ORF1a and ORF1ab that code for polyprotein 1a and 1ab, respectively, which are subsequently proteolytically processed to 16 non-structural proteins (NSP) by the two virally encoded proteases (NSP3 and NSP5) and execute replication and transcription of the viral genome (reviewed in¹⁸). The 3′ one-third of the genome include the viral structural and accessory proteins. SARS-CoV-2 particles are composed of four structural proteins including Spike (S), Envelope (E), Membrane (M), and Nucleocapsid (N)¹⁹⁻²¹. The S protein mediates viral entry and fusion by binding the ACE2 receptor on cells and is the subject of evolutionary selection to evade neutralization by vaccine- and infection-elicited antibodies⁵. The viral accessory proteins have diverse functions contributing to infectivity, replication, and pathogenesis and other unknown functions (reviewed in²²).

To study SARS-CoV-2 attenuation and the full range of mutations along the Omicron genome, it is necessary to construct full-length recombinant viruses or near full-length replicons^{12,23}. Constructing SARS-CoV-2 recombinant clones in a timely manner is challenging due to the length of the viral genome (30 kb) and toxic viral sequences that limit standard molecular cloning strategies. Several approaches have been reported for generating SARS-CoV-2 infectious clones. These include the synthetic circular polymerase extension reaction (CPER) approach^{24,25}, the ligation of synthetic fragments using unique restriction enzymes in the SARS-CoV-2 genome²⁶⁻²⁸, and ligation of synthetic or cloned fragments using type IIs restriction enzymes²⁹⁻³¹. While the CPER approach is fast, it suffers from a potentially heterogeneous non-clonal population of sequences that can arise during synthesis or PCR amplification. This therefore requires additional plaque purification of viruses to ensure homogeneity, which adds time and effort to accessing these sequences. While utilization of unique restriction sites in the genome can facilitate genome cloning and assembly, the dependence on specific restriction sites renders generation and manipulation of recombinant viruses inflexible. In addition, the stepwise ligation of fragments (in most cases >5 fragments) requires long incubation (typically 2- or 3-fragment ligation step/day) and purification steps and results in low yields of the full-length ligated genome. Therefore, currently available methods remain challenging to utilize in the context of rapid characterization of emerging SARS-CoV-2 variants.

To overcome these limitations, a plasmid-based viral genome assembly and rescue (pGLUE) was developed, a novel method to rapidly generate full-length SARS-CoV-2 recombinant infectious clones and near full-length non-infectious replicons to interrogate the Omicron life cycle. pGLUE takes advantage of type IIs restriction enzymes that cleave outside their recognition sequences and when combined with a ligase and temperature cycling-known as the Golden Gate Assembly method—can be used to seamlessly digest and ligate viral sequences in a rapid fashion. While previous studies utilized type IIs restriction enzymes²⁹⁻³¹ to release viral sequences from plasmids, none have so far taken full advantage of the Golden Gate Assembly method to carry out rapid ligation of the entire genome.

Using pGLUE, naturally occurring Delta- and Omicron mutations were examined in recombinant infectious clones and also designed a replicon system to specifically study viral RNA replication independently of Spike. It was found that Omicron mutations in NSP4-6 attenuate viral RNA replication compared with the Delta variant. These results indicate that the cost for viral adaptation is broader than previously thought.

### Materials and Methods

### Cells

BHK21 were obtained from ATCC (CCL-10) and cultured in DMEM (Corning) supplemented with 10% fetal bovine serum (FBS) (GeminiBio), 1× glutamine (Corning), and 1× penicillin-streptomycin (Corning) at 37° C., 5% CO₂. Calu3 cells were obtained from ATCC and cultured in AdvancedMEM (Gibco) supplemented with 2.5% FBS, 1× GlutaMax, and 1× penicillin-streptomycin at 37° C. and 5% CO₂. Vero cells stably overexpressing human TMPRSS2 (Vero-TMPRSS2) (gifted from the Whelan 1ab⁶⁷), were grown in DMEM with 10% FBS, 1× glutamine, 1× penicillin-streptomycin at 37° C. and 5% CO₂. Vero cells stably co-expressing human ACE2 and TMPRSS2 (Vero-ACE2/TMPRSS2) (gifted from A. Creanga and B. Graham at NIH) were maintained in Dulbecco's Modified Eagle medium (DMEM; Gibco) supplemented with 10% FBS, 100 μg/mL penicillin and streptomycin, and 10 μg/mL of puromycin at 37° C. and 5% CO₂.

### Infectious Clone Preparation

To enable this rapid cloning strategy, the SARS-CoV-2 genome was divided into 10 fragments that correspond to different coding regions of the genome. The fragments were cloned into a pUC19-based vector with the bidirectional tonB terminator upstream and the T7Te and rrnB T1 terminators downstream of the SARS-CoV-2 sequence. Prior to assembly, the fragments were PCR amplified and cleaned. To enable assembly of the full-length SARS-CoV-2 genome using BsaI-mediated Golden Gate assembly, the two BsaI sites in the genome (WA1 nt 17966 and nt 24096) were eliminated by introducing the following synonymous mutations (WA1 nt C17976T and nt C24106T) in fragments F6 and F8, respectively. The pBAC vector that can handle the full-length genome was purchased from Lucigen (cat #42032-1). This vector was modified to include a CMV promoter, T7 promoter, BsaI sites, an HDVrz and SV40 polyA. The BsaI site at nt 2302 was mutated (C2307T) to allow use in the BsaI-mediated Golden Gate assembly. For the Golden Gate assembly, the 10 fragments and the pBAC vector were mixed in stoichiometric ratios in 1× T4 DNA ligase buffer (25 μL reaction volume). To the mixture was added BsaI HF v2 (1.5 μL) and Hi-T4 DNA ligase (2.5 μL). The assembly was performed as follows in a thermal cycler: 30 cycles of 37° C. for 5 min, followed by 16° C. for 5 min. Then the reaction was incubated at 37° C. for 5 min and 60° C. for 5 min. 1 μL of the reaction was electroporated into EPI300 cells and plated onto LB+chloramphenicol plates and grown at 37° C. for 24 hours. Colonies were picked and cultured in LB30 medium+12.5 μg/mL of chloramphenicol for 12 hours at 37° C. 1 mL of the culture was diluted to 100 mL of LB30 medium+12.5 μg/mL of chloramphenicol for 3-4 hours. The culture was diluted again to 400 mL of LB30 medium+12.5 μg/mL of chloramphenicol+1× Arabinose induction solution (Lucigen) for overnight. The pBAC infectious clone plasmid was extracted and purified using NucleoBond Xtra Maxi prep kit (Macherey-Nagel). All plasmids constructed in the study will be available via Addgene.

### In Vitro Transcribed RNA Preparation

20 μg of the pBAC infectious clone plasmid was digested with Sa1I and SbfI for at least 3 hours at 37° C. in a 50-μL reaction. The digest was diluted to 500 μL with DNA lysis buffer (0.5% SDS, 10 mM Tris, pH 8, 10 mM EDTA, and 10 mM NaCl) and 5 μL of proteinase K was added. The mixture was incubated at 50° C. for 1 hour. The DNA was extracted with phenol and precipitated with ethanol. 2 μg of digested DNA was used to set up the IVT reactions according to the manufacturer's instructions for both the HiScribe and the mMessage mMachine kits except for the incubation times as indicated (FIG. 1E). The mMessage mMachine Kit was used to generate the RNA for all infectious clone experiments. After the IVT reaction, the RNA was extracted with RNAstat60 and precipitated with isopropanol, according to the manufacturer's instructions. To generate N IVT RNA, the exact procedure above was followed, except that the plasmid was digested with Sa1I only and the IVT reaction was run for 2 hours at 37° C.

### Infectious Clone Virus Rescue

To generate the RNA-launched SARS-CoV-2, the purified infectious clone RNA (10 μg) was mixed with N RNA (5 μg) and electroporated into 5×10⁶ BHK21 cells. The cells were then layered on top of Vero-ACE2/TMPRSS2 cells in a T75 flask (FIG. 2A). After development of cytopathic effect, the virus was propagated onto Vero-ACE2/TMPRSS2 to achieve high titer. To generate the DNA-launched SARS-CoV-2, the pBAC SARS-CoV-2 construct was directly cotransfected with N expression construct into BHK21 cells in six-well plate (FIG. 2A). After 3 days post-transfection, the supernatant was collected and used to infect Vero-ACE2/TMPRSS2 cells and passaged further to achieve high titer.

### SARS-CoV-2 Replicon Assay

Plasmids harboring the full SARS-CoV-2 sequence except for spike (1 μg) were transfected into BHK21 cells along with nucleocapsid and spike expression vectors (0.5 μg each) in 24-well plate using X-tremeGENE 9 DNA transfection reagent (Sigma Aldrich) according to manufacturer's protocol. The supernatant was replaced with fresh growth medium 12-16 hours post transfection. The supernatant containing single-round infectious particles was collected and 0.45 μm-filtered 72 hours post transfection. The supernatant was subsequently used to infect Vero-ACE2/TMPRSS2 cells (in 96-well plate) or Calu3 cells (in 24-well plate). The medium was refreshed 12-24 hours post infection. To measure luciferase activity, an equal volume of supernatant from transfected cells or infected cells was mixed with Nano-Glo luciferase assay buffer and substrate and analyzed on an Infinite M Plex plate reader (Tecan).

### SARS-CoV-2 Virus Culture and Plaque Assay

SARS-CoV-2 variants B.1.617.2 (BEI NR-55611) and B.1.1.529 (California Department of Health) were propagated on Vero-ACE2/TMPRSS2 cells, sequence verified, and were stored at −80° C. until use. The virus infection experiments were performed in a Biosafety Level 3 laboratory. For plaque assays, tissue homogenates and cell supernatants were analyzed for viral particle formation for in vivo and in vitro experiments, respectively. Briefly, Vero-ACE2/TMPRSS2 cells were plated and rested for at least 24 hours. Serial dilutions of inoculate of homogenate or supernatant were added on to the cells. After the 1-hour absorption period, 2.5% Avicel (Dupont, RC-591) was overlaid. After 72 hours, the overlay was removed, the cells were fixed in 10% formalin for one hour and stained with crystal violet for visualization of plaque formation.

### Analysis of Viral Sequences

Viral sequences were downloaded from the GISAID database and analyzed for mutations utilizing the Geneious Prime software version 2022.2.1. The GISAID mutation analysis tool was utilized to quickly filter for recombinants containing specific mutations prior to download.

### Real-Time Quantitative Polymerase Chain Reaction (RT-qPCR)

RNA was extracted from cells, supernatants, or tissue homogenates using RNA-STAT-60 (AMSBIO, CS-110) and the Direct-Zol RNA Miniprep Kit (Zymo Research, R2052). RNA was then reverse transcribed to cDNA with iScript cDNA Synthesis Kit (Bio-Rad, 1708890). qPCR reaction was performed with cDNA and SYBR Green Master Mix (Thermo Fisher Scientific) using the CFX384 Touch Real-Time PCR Detection System (Bio-Rad). N gene primer sequences are: Forward 5′ AAATTTTGGGGACCAGGAAC 3′ (SEQ ID NO: 1); Reverse 5′ TGGCACCTGTGTAGGTCAAC 3′. (SEQ ID NO: 2) The tenth fragment of the infectious clone plasmid was used as a standard for N gene quantification by RT-qPCR.

### K18-hACE2 Mouse Infection Model

All protocols concerning animal use were approved (AN169239-01C) by the Institutional Animal Care and Use committees at the University of California, San Francisco and Gladstone Institutes and conducted in strict accordance with the National Institutes of Health Guide for the Care and Use of Laboratory Animal. Mice were housed in a temperature- and humidity-controlled pathogen-free facility with 12-hour light/dark cycle and ad libitum access to water and standard laboratory rodent chow. Briefly, the study involved intranasal infection (1×10⁴ PFU) of 6-8-week-old K18-hACE2 mice with Delta (DNA, RNA, and patient isolate). A total of 5 animals were infected for each variant and euthanized at 2 days post-infection. The lungs were processed for further analysis of virus replication.

### Cellular Infection Studies

Calu3 cells were seeded into 12-well plates. Cells were rested for at least 24 hours prior to infection. At the time of infection, medium containing viral inoculum was added on the cells. One hour after addition of inoculum, the medium was replaced with fresh medium. The supernatant was harvested at 24-, 48-, and 72-hours post-infection for downstream analysis.

### Results

### Golden Gate Assembly Enables Rapid Cloning of SARS-CoV-2 Variants

To determine which parts of the Omicron genome contribute to the attenuated phenotype, pGLUE (plasmid-based viral genome assembly and rescue): a rapid method to generate SARS-CoV-2 molecular clones with Golden Gate assembly (FIG. 1A) was designed and developed. The SARS-CoV-2 genome was divided into 10 fragments to enable quick and reliable cloning of mutations. The fragments were designed rationally to cover the SARS-CoV-2 ORFs and enable easy construction of chimeric viruses. The fragments were assembled along with a bacterial artificial chromosome (BAC) vector to enable growth of toxic sequences within the SARS-CoV-2 genome in bacteria²⁹⁻³¹. At the 5′ end, the vector bears T7 and CMV promoters with the T7 promoter nested in between the TATA box sequence of the CMV promoter and the SARS-CoV-2 RNA transcription start site. This is to enable efficient and seamless DNA- and RNA-launch of viruses. The 3′ end of the destination vector contained a hepatitis delta ribozyme (HDVrz) and SV40 polyA sequence for efficient and homogenous 3′ RNA processing.

The Golden Gate assembly reaction is efficient and proceeds almost to completion within 30 cycles (˜6 hours) as indicated by the slower migrating band (FIG. 1B). Sequencing of the assembled constructs for the WA1, Delta, and Omicron variants showed over 80% of the colonies were correctly assembled and free of any mutations (FIG. 1C). In addition, preparation of the construct in high quantity and quality was demonstrated by relatively high abundance of all expected plasmid fragments (FIG. 1D). Two different kits were utilized and optimized for production of full-length SARS-CoV-2 RNA as indicated by the co-migration of the RNA band with the template DNA band (FIG. 1E). The HiScribe kit was more efficient in producing the full-length RNA than the mMessage mMachine kit (2 hours vs overnight reaction, respectively), but it had lower total yield of RNA (10 μg/reaction vs>100 μg/reaction, respectively).

Cloning of a full-length variant from sequence to sequenced plasmid can be achieved on average in 1 week. The assembled construct can then be transfected directly into appropriate target cells for recovery of infectious virus or can be subjected to in vitro transcription with T7 polymerase followed by electroporation into cells and virus rescue (FIG. 2A). Rescue of DNA- and RNA-launched viruses on average and depending on a given variant's infectivity can be achieved in 1-2 weeks. To test the replication kinetics of recombinant viruses, Delta variant derived from DNA or RNA was cloned and rescued. These viruses were compared with a patient-derived Delta variant in cell culture and animal models of infection. The patient-derived and de novo constructed recombinant viruses had similar plaque morphology (FIG. 2B), replication kinetics in Vero-TMPRSS2 and Calu3 cells (FIG. 2C) and showed similar viral loads in K18-hACE2 mice (FIG. 2D). Thus, the pGLUE method is robust and produces viruses that are comparable to patient-derived viruses.

### Omicron Mutations in Spike and ORF1ab Reduce Viral Particle Production and Intracellular RNA Levels

Using pGLUE, several recombinant clones of the Delta and Omicron variants were constructed (FIG. 3A). For the Delta and Omicron variants, the mutations selected were representative of >90% of all Delta and Omicron sequences on the GISAID database as of January 2022. In addition, two naturally occurring viruses were focused on: 1) “Deltacron” which harbors the Omicron Spike ORF within the Delta variant³²⁻³⁴ and 2) a virus harboring the Omicron ORF1ab within the Delta variant also found in the GISAID database. Full-length genomes were constructed using pGLUE and labeled Delta-OmicronS and Omicron-Delta, respectively (FIG. 3A). The resulting viruses were propagated in Vero ACE2 TMPRSS2 cells, and infectious particle production was measured in plaque assays (FIG. 3B).

Significant differences in plaque morphology were observed (FIG. 3B). The Delta variant produced the largest plaque sizes of the tested viruses while plaques produced by Omicron were the smallest. Similar data were recently reported for Delta and Omicron Spike and point to the Omicron RBD as the mediator of the smaller plaque size³⁵. Delta-OmicronS produced small plaques, which were slightly larger than that of the Omicron variant. This indicates that receptor binding and fusion capabilities are largely endowed by the Spike protein and that the Omicron Spike protein has reduced fusogenic properties compared to Delta's. Interestingly, Omicron-Delta produced smaller plaques than the Delta variant pointing to negative contributions of the Omicron ORF1ab to this phenotype.

Next, the growth kinetics of the different viruses were determined at 24, 48 and 72 hours in Calu3 cells infected at a multiplicity of infection (m.o.i.) of 0.1 (FIGS. 3C and 3D). Of note, the presence of the Omicron Spike ORF in the Delta variant attenuated particle production significantly. This confirms that Spike mutations play a significant role in tuning Omicron's replicative fitness³⁵⁻³⁷. However, the presence of Omicron ORF1ab in Delta also significantly reduced infectious particle production, indicating that mutations in ORF1ab contribute to Omicron attenuation. The same was observed when intracellular RNA levels were determined by reverse transcription and quantitative PCR (FIG. 3D). Collectively, these data indicate that mutations in Spike and ORF1ab contribute to reduced viral fitness of the Omicron variant in cell culture.

### Spike-Independent Attenuation of Omicron

To define further Spike-independent differences between Omicron and Delta, a replicon system lacking the Spike protein was constructed (FIGS. 4A and 4B). This system does not produce viral particles unless Spike is provided in trans, allowing only a single round of infection. Briefly, the entire Spike coding sequence was replaced with the one for secreted nanoluciferase (nLuc) and enhanced green fluorescent protein (EGFP). Of note, only the luciferase readout in this study because of its sensitivity and dynamic range. Transfection of the replicon construct successfully launches viral genome replication in transfected cells as indicated by detectable luciferase activity in the cell supernatant (FIG. 4C). Interestingly, the Delta replicon produced fivefold higher luciferase signal than the Omicron replicon (FIG. 4C), underscoring that non-Spike mutations are contributing to Omicron attenuation. No significant luciferase activity was observed when the supernatant from these cultures was transferred to permissive cells (FIG. 4D), confirming the absence of infectious particle production from the transfected replicon construct. When the appropriate Spike vector was cotransfected with the replicon construct production of infectious particles occurred as indicated by luciferase activity in both transfected and infected cells (FIGS. 4C and 4D). A Spike vector with naturally occurring Delta mutations (FIG. 3A) was used to enhance single round infection efficiencies⁹.

Surprisingly, transfection of increasing amounts of the Spike expression construct while maintaining a constant amount of the replicon construct led to increasing luciferase activity in both transfected and infected cells (FIGS. 4C and 4D). Previous reports on particle assembly using only viral structural proteins suggested that only trace amounts of Spike are necessary for particle assembly and that higher amounts led to lower particle assembly^{38,39}. This indicates that other viral proteins, which were not present in these previous experiments, are important in Spike processing or mediate critical steps in the assembly process. Regardless of the Spike amount transfected, the Omicron variant consistently performed worse, as shown by reduced luciferase signal, compared with the Delta variant, in both transfected and infected cells (FIGS. 4C and 4D). These results support the model that non-Spike Omicron mutations are attenuating viral RNA replication.

To map the contribution of non-Spike Omicron mutations on viral RNA replication within the Omicron genome, several replicon constructs were constructed with tiled segments of the Omicron genome replaced with those in Delta. These replicon constructs were transfected along with the appropriate Spike vectors to assess the contribution of Omicron mutations on viral RNA replication, again only in single-round infection experiments. Delta and Omicron replicons were used as controls and showed the expected difference in transfected and infected cells (FIGS. 4E and 4F). Replacement of Omicron NSP4-6 with Delta's significantly restored the luciferase signal in transfected and infected cells (FIGS. 4E and 4F), indicating that mutations in these proteins contribute to Spike-independent attenuation of Omicron. A significant increase was also observed for NSP10-13 and NSP14 substitutions (FIGS. 4E and 4F).

These results indicate that potentially multiple functions of nonstructural proteins are impaired in Omicron, including double membrane vesicle formation mediated by NSP4 and 6, viral polyprotein proteolysis mediated by NSP5, RNA replication mediated by NSP10-13, and RNA proofreading mediated by NSP14. Of note, the replicon where accessory proteins ORF8-10 from Delta were tested in an Omicron background, produced similar luciferase signals, compared with the Omicron variant in transfected cells (FIG. 4E), but the signal was significantly reduced in infected cells (FIG. 4F). This construct also encompasses the N protein. The Omicron and Delta N proteins perform similarly with regards to particle assembly in the context of virus-like particles³⁸, thereby suggesting a possible role for ORF8 Delta mutations, specifically DF119-120del, in particle assembly. Collectively, these findings confirm that non-Spike mutations in Omicron are attenuating viral genome replication and also hint to additional functions in particle assembly.

### Attenuating Mutations are Subject of Evolutionary Pressure Across Omicron Isolates

To examine mutational “hot spots” across naturally existing sequences before and after the occurrence of Omicron, the entropy of nucleotide changes were analyzed across the SARS-CoV-2 genome of subsampled sequences since the beginning of the pandemic⁴⁰. The sequences were stratified by date to distinguish between evolutionary tendencies before (December 2019 to November 2021) and after (January 2022 to August 2022) the emergence of the Omicron variant (FIGS. 5A and 5B). The month of December 2021 was excluded from the analysis as both Delta and Omicron sequences were abundant, which may skew the analysis. The normalized Shannon entropy calculated per nucleotide indicates uncertainty that the nucleotide will remain unchanged within the given sample of sequences. Therefore, higher entropy indicates higher diversity and mutational activity given a set of sequences at a certain time point.

Comparison of the entropies across the first two-thirds of the genome encompassing ORF1ab revealed marked differences between pre- and post-Omicron sequences (FIG. 5A) and indicated a change in the evolutionary path of SARS-CoV-2 after the emergence of Omicron. While the positions with high entropy (>0.4) were sparse and spread relatively evenly across ORF1ab prior to Omicron emergence, a pronounced clustering of mutations was apparent for NSP4 after Omicron's emergence. In fact, the NSP4 locus has seen most mutations within ORF1ab in evolved Omicron variants, such as BA.2 (3 nonsynonymous mutations) and BA.5 (2 nonsynonymous mutations). NSP3 sequences technically show five mutations relative to ancestral Omicron, but three of these are revertants to WA1 sequences. Similarly, the NSP6 locus has one new mutation and a reverting mutation. Other NSPs show significantly less mutations in evolved Omicron variants including one mutation each in NSP1, 13, and 15. Collectively, the results underscore a role of NSP4 and possibly NSP5 and 6 in Omicron attenuation.

### DISCUSSION

The data provide both technical and biological advances. Technically, a novel cloning system was built with rational fragment design and single-pot ligation (pGLUE) that allows molecular interrogation of entire SARS-CoV-2 genomes within days. Biologically, it was determined that Omicron mutations in ORF1ab lower viral fitness with previously unappreciated contributions of NSP4-6.

Generating molecular viral clones is important, given the delay with obtaining regionally occurring patient isolates, the risk of undesired mutations during prolonged viral propagation, and the existence of toxic sequences that limit standard molecular cloning strategies. Using pGLUE, viral variant genomes were routinely designed and produced within a week. This efficiency enables an art worker to address real-world changes in viral evolution with respect to all lifecycle steps. pGLUE is different from previous methods²⁴⁻³¹ in that: 1) it employs rational fragment design eliminating issues with toxic sequences in bacteria and enabling rapid virus and replicon generation; 2) it is plasmid-based and therefore has inherent reliability and accuracy; and 3) it takes full advantage of Golden Gate assembly to perform rapid single-pot ligation of the entire genome in less than six hours. The developed method is robust and will continue to provide valuable insight into the molecular mechanisms of the SARS-CoV-2 lifecycle beyond what is presented in this study.

A large body of evidence has characterized the Omicron Spike protein and showed that it favors TMPRSS2-independent endosomal entry^{9,41,42}, has poor fusogenicity⁴², and escapes neutralization by many antibodies⁴²⁻⁴⁵. Furthermore, studies using chimeric viruses bearing different Spike proteins showed that Spike is a major determinant of the Omicron attenuated replicative phenotype³⁵⁻³⁷. The results (FIG. 3) confirm these findings and underscore the critical role that the Spike protein plays in determining viral fitness and skewing viral adaptation towards immune escape.

Less work has been done so far to investigate the impact of the Omicron mutations outside of the Spike protein. Previously, a Spike-independent attenuation of the Omicron variant in animals has been reported^{46,47}. The data define a new role of ORF1ab Omicron mutations, namely in NSP4-6, in the attenuation process, implicating reduced RNA replication and polyprotein processing in the adaptation process. The precise molecular mechanism and the individual mutations involved need to be further defined, but the entropy calculations confirm that NSP4-6 are undergoing rapid mutagenesis in the post-Omicron era. NSP4 forms a complex with NSP3 and 6 and together anchors viral replication complexes onto double-membrane vesicles in the cytoplasm that protect the replicating viral genomes⁴⁸. NSP5 is a cysteine protease responsible for processing the viral polyprotein at sites between NSP4-16. The data suggest that NSP4-6 of Omicron are less efficient in supporting RNA replication than Delta NSP4-6 and underscore the importance of membrane rearrangement and protease function in viral fitness.

Collectively, the findings demonstrate that not only Spike, but also non-Spike mutations of the Omicron variant are attenuating. It remains unclear how these mutations came to arise together in Omicron given their low composite fitness. Several studies have suggested that Omicron could have emerged due to epistatic interactions that may allow for the emergence of mutations not seen in other variants or that are very rare⁴⁹⁻⁵¹. The low intra-host evolution for SARS-CoV-2 and relatively limited transmission bottleneck⁵²⁻⁵³ suggest that Omicron may have evolved in chronically infected patients where the virus can cross through fitness valleys that may not be possible in an acute infection⁴⁹. Interestingly, Omicron mutations in Spike (K417N and L981F) occur within conserved MHC-I-restricted CD8⁺ T-cell epitopes that may destabilize MHC-I complexes⁵⁴, indicating that T-cell immunity is an additional driver of SARS-CoV-2 evolution as in other viruses⁵⁵⁻⁵⁷.

An advantage of the findings is that they can help generate candidates for live attenuated SARS-CoV-2 vaccines in the future⁵⁸. A potential caveat is the introduction of antivirals such as Paxlovid, which targets specifically NSP5 and may lead to development of selective resistance mutations⁵⁹⁻⁶¹. The diversity analysis of pre- and post-Omicron mutations indicates that the virus continues to evolve, which carries the risk of reversion of the attenuating mutations in Omicron.

This is supported by recent reports on the enhanced infectivity and neutralization escape of Omicron-evolved subvariants⁶²⁻⁶⁶. The ability to rapidly characterize full-length viral sequences is therefore increasingly valuable and will bring insight into the evolutionary path, viral fitness, expected pathogenicity as well as vaccine and antiviral medication responsiveness of emerging subvariants.

### Example II

The COVID-19 pandemic continues to be a major public health issue worldwide. Since the beginning of the pandemic, unprecedented scientific efforts were taken to generate antivirals against SARS-CoV-2. To build on these efforts and accelerate the development of novel antivirals, it is necessary to develop robust antiviral assays amenable to high-throughput screening. To that end, two reporter luciferase- and fluorescence-based viruses with distinct readouts that can serve as secondary screens for each other were generated. Briefly, these reporter viruses are used to infect cells that have been treated with potential antiviral compounds and the reporter activity is read out over time post-infection (FIGS. 6 and 7). These reporter viruses have been validated utilizing approved as well as investigational antivirals (FIGS. 6 and 7). These viruses are currently being utilized for high throughput screening of potential antivirals targeting several viral proteins.

### Example III

SARS-CoV-2 has caused a worldwide pandemic and the origin of the virus has not been clearly demonstrated yet. One of the earliest detected ancestors of SARS-CoV-2 is a bat SARS-related coronavirus named RaTG13. Although RaTG13 has over 1000 mutations relative to SARS-CoV-2, one of the mutations of interest is in Orf9b which is a viral protein involved in innate immune antagonism. To understand the role of this mutation in the viral lifecycle, the invention was utilized to construct Spike replicons of both SARS-CoV-2 and RaTG13 as well as a mutant RaTG13 Orf9b I72T containing the SARS-CoV-2 amino acid residue at that site (FIG. 8). It was found that RaTG13 replicates quite lower than SARS-CoV-2 in VAT cells but replicates similarly in bat cells. Interestingly, the Orf9b mutant replicated somewhat similarly to ancestral RaTG13. These data suggest that RaTG13 likely does not replicate efficiently in human cells and some of the mutations acquired by SARS-CoV-2 may have been critical for adaptation to humans. Further cell models of infection are likely necessary to understand the role of Orf9b in RaTG13 infection as well as its impact on innate immune antagonism in bat and human cells.

### BIBLIOGRAPHY

1. WHO, Vol. 2022 (World Health Organization, 2022).
2. Davies, N. G. et al. Estimated transmissibility and impact of SARS-CoV-2 lineage B.1.1.7 in England. Science 372 (2021).
3. Liu, Y. & Rocklov, J. The reproductive number of the Delta variant of SARS-CoV-2 is far higher compared to the ancestral SARS-CoV-2 virus. J Travel Med 28 (2021).
4. Liu, Y., Gayle, A. A., Wilder-Smith, A. & Rocklov, J. The reproductive number of COVID-19 is higher compared to SARS coronavirus. J Travel Med 27 (2020).
5. Perez-Then, E. et al. Neutralizing antibodies against the SARS-CoV-2 Delta and Omicron variants following heterologous CoronaVac plus BNT162b2 booster vaccination. Nat Med 28, 481-485 (2022).
6. Wolter, N. et al. Early assessment of the clinical severity of the SARS-CoV-2 omicron variant in South Africa: a data linkage study. Lancet 399, 437-446 (2022).
7. Garrett, N. et al. High Asymptomatic Carriage With the Omicron Variant in South Africa. Clin Infect Dis 75, e289-e292 (2022).
8. Vihta, K. D. et al. Omicron-associated changes in SARS-CoV-2 symptoms in the United Kingdom. Clin Infect Dis (2022).
9. Meng, B. et al. Altered TMPRSS2 usage by SARS-CoV-2 Omicron impacts infectivity and fusogenicity. Nature 603, 706-714 (2022).
10. Suzuki, R. et al. Attenuated fusogenicity and pathogenicity of SARS-CoV-2 Omicron variant. Nature 603, 700-705 (2022).
11. Shuai, H. et al. Attenuated replication and pathogenicity of SARS-CoV-2 B.1.1.529 Omicron. Nature 603, 693-699 (2022).
12. Mautner, L. et al. Replication kinetics and infectivity of SARS-CoV-2 variants of concern in common cell culture models. Virol J 19, 76 (2022).
13. Halfmann, P. J. et al. SARS-CoV-2 Omicron virus causes attenuated disease in mice and hamsters. Nature 603, 687-692 (2022).
14. McMahan, K. et al. Reduced pathogenicity of the SARS-CoV-2 omicron variant in hamsters. Med (N Y) 3, 262-268 e264 (2022).
15. Yuan, S. et al. The SARS-CoV-2 Omicron (B.1.1.529) variant exhibits altered pathogenicity, transmissibility, and fitness in the golden Syrian hamster model. bioRxiv (2022).
16. Rochman, N. D. et al. Ongoing global and regional adaptive evolution of SARS-CoV-2. Proc Natl Acad Sci USA 118 (2021).
17. Coronaviridae Study Group of the International Committee on Taxonomy of, V. The species Severe acute respiratory syndrome-related coronavirus: classifying 2019-nCoV and naming it SARS-CoV-2. Nat Microbiol 5, 536-544 (2020).
18. Jin, Y. et al. Genome-Wide Analysis of the Indispensable Role of Non-structural Proteins in the Replication of SARS-CoV-2. Front Microbiol 13, 907422 (2022).
19. Ke, Z. et al. Structures and distributions of SARS-CoV-2 spike proteins on intact virions. Nature 588, 498-502 (2020).
20. Yao, H. et al. Molecular Architecture of the SARS-CoV-2 Virus. Cell 183, 730-738 e713 (2020).
21. Mendonca, L. et al. Correlative multi-scale cryo-imaging unveils SARS-CoV-2 assembly and egress. Nat Commun 12, 4629 (2021).
22. Redondo, N., Zaldivar-Lopez, S., Garrido, J. J. & Montoya, M. SARS-CoV-2 Accessory Proteins in Viral Pathogenesis: Knowns and Unknowns. Front Immunol 12, 708264 (2021).
23. Mlcochova, P. et al. SARS-CoV-2 B.1.617.2 Delta variant replication and immune evasion. Nature 599, 114-119 (2021).
24. Torii, S. et al. Establishment of a reverse genetics system for SARS-CoV-2 using circular polymerase extension reaction. Cell Rep 35, 109014 (2021).
25. Amarilla, A. A. et al. A versatile reverse genetics platform for SARS-CoV-2 and other positive-strand RNA viruses. Nat Commun 12, 3431 (2021).
26. Rihn, S. J. et al. A plasmid DNA-launched SARS-CoV-2 reverse genetics system and coronavirus toolkit for COVID-19 research. PLoS Biol 19, e3001091 (2021).
27. Ricardo-Lax, I. et al. Replication and single-cycle delivery of SARS-CoV-2 replicons. Science 374, 1099-1106 (2021).
28. Ye, C. et al. Rescue of SARS-CoV-2 from a Single Bacterial Artificial Chromosome. mBio 11 (2020).
29. Ju, X. et al. A novel cell culture system modeling the SARS-CoV-2 life cycle. PLoS Pathog 17, e1009439 (2021).
30. Xie, X. et al. Engineering SARS-CoV-2 using a reverse genetic system. Nat Protoc 16, 1761-1784 (2021).
31. Xie, X. et al. An Infectious cDNA Clone of SARS-CoV-2. Cell Host Microbe 27, 841-848 e843 (2020).
32. Colson, P. et al. Culture and identification of a “Deltamicron” SARS-CoV-2 in a three cases cluster in southern France. J Med Virol 94, 3739-3749 (2022).
33. Lacek, K. A. et al. SARS-CoV-2 Delta-Omicron Recombinant Viruses, United States. Emerg Infect Dis 28, 1442-1445 (2022).
34. SIMON-LORIERE E et al. Rapid characterization of a Delta-Omicron SARS-CoV-2 recombinant detected in Europe. Research Square (2022).
35. Barut, G. T. et al. The spike gene is a major determinant for the SARS-CoV-2 Omicron-BA.1 phenotype. Nat Commun 13, 5929 (2022).
36. Yamasoba, D. et al. Virological characteristics of the SARS-CoV-2 Omicron BA.2 spike. Cell 185, 2103-2115 e2119 (2022).
37. Peacock, T. P. et al. The altered entry pathway and antigenic distance of the SARS-CoV-2 Omicron variant map to separate domains of spike protein. bioRxiv (2022).
38. Syed, A. M. et al. Rapid assessment of SARS-CoV-2-evolved variants using virus-like particles. Science 374, 1626-1632 (2021).
39. Chaturvedi, S. et al. Identification of a therapeutic interfering particle-A single-dose SARS-CoV-2 antiviral intervention with a high barrier to resistance. Cell 184, 6022-6036 e6018 (2021).
40. Hadfield, J. et al. Nextstrain: real-time tracking of pathogen evolution. Bioinformatics 34, 4121-4123 (2018).
41. Willett, B. J. et al. SARS-CoV-2 Omicron is an immune escape variant with an altered cell entry pathway. Nat Microbiol 7, 1161-1179 (2022).
42. Du, X. et al. Omicron adopts a different strategy from Delta and other variants to adapt to host. Signal Transduct Target Ther 7, 45 (2022).
43. Cao, Y. et al. Omicron escapes the majority of existing SARS-CoV-2 neutralizing antibodies. Nature 602, 657-663 (2022).
44. Cele, S. et al. Omicron extensively but incompletely escapes Pfizer BNT162b2 neutralization. Nature 602, 654-656 (2022).
45. Zhang, L. et al. The significant immune escape of pseudotyped SARS-CoV-2 variant Omicron. Emerg Microbes Infect 11, 1-5 (2022).
46. Liu, S., Selvaraj, P., Sangare, K., Luan, B. & Wang, T. T. Spike protein-independent attenuation of SARS-CoV-2 Omicron variant in laboratory mice. Cell Rep 40, 111359 (2022).
47. Chen, D. Y. et al. Role of spike in the pathogenic and antigenic behavior of SARS-CoV-2 BA.1 Omicron. bioRxiv (2022).
48. Ricciardi, S. et al. The role of NSP6 in the biogenesis of the SARS-CoV-2 replication organelle. Nature 606, 761-768 (2022).
49. Harari, S. et al. Drivers of adaptive evolution during chronic SARS-CoV-2 infections. Nat Med 28, 1501-1508 (2022).
50. Fooladinezhad, H. et al. SARS-CoV-2 NSP3, NSP4 and NSP6 mutations and Epistasis during the pandemic in the world: Evolutionary Trends and Natural Selections in Six Continents. medRxiv (2022).
51. Martin, D. P. et al. Selection analysis identifies unusual clustered mutational changes in Omicron lineage BA.1 that likely impact Spike function. bioRxiv (2022).
52. Lythgoe, K. A. et al. SARS-CoV-2 within-host diversity and transmission. Science 372 (2021).
53. Braun, K. M. et al. Acute SARS-CoV-2 infections harbor limited within-host diversity and transmit via tight transmission bottlenecks. PLoS Pathog 17, e1009849 (2021).
54. Agerer, B. et al. SARS-CoV-2 mutations in MHC-I-restricted epitopes evade CD8(+) T cell responses. Sci Immunol 6 (2021).
55. Pircher, H. et al. Viral escape by selection of cytotoxic T cell-resistant virus variants in vivo. Nature 346, 629-633 (1990).
56. Goulder, P. J. et al. Evolution and transmission of stable CTL escape mutations in HIV infection. Nature 412, 334-338 (2001).
57. Cox, A. L. et al. Cellular immune selection with hepatitis C virus persistence in humans. J Exp Med 201, 1741-1752 (2005).
58. Liu, Y. et al. A live-attenuated SARS-CoV-2 vaccine candidate with accessory protein deletions. Nat Commun 13, 4337 (2022).
59. Jochmans, D. et al. The substitutions L50F, E166A and L167F in SARS-CoV-2 3CLpro are selected by a protease inhibitor <em>in vitro</em> and confer resistance to nirmatrelvir. bioRxiv (2022).
60. Hu, Y. et al. Naturally occurring mutations of SARS-CoV-2 main protease confer drug resistance to nirmatrelvir. bioRxiv (2022).
61. Moghadasi, S. A. et al. Transmissible SARS-CoV-2 variants with resistance to clinical protease inhibitors. bioRxiv (2022).
62. Uraki, R. et al. Characterization and antiviral susceptibility of SARS-CoV-2 Omicron BA.2. Nature 607, 119-127 (2022).
63. Kimura, I. et al. Virological characteristics of the novel SARS-CoV-2 Omicron variants including BA.2.12.1, BA.4 and BA.5. bioRxiv (2022).
64. Tuekprakhon, A. et al. Antibody escape of SARS-CoV-2 Omicron BA.4 and BA.5 from vaccine and BA.1 serum. Cell 185, 2422-2433 e2413 (2022).
65. Cao, Y. et al. BA.2.12.1, BA.4 and BA.5 escape antibodies elicited by Omicron infection. Nature 608, 593-602 (2022).
66. Wang, Q. et al. Antigenic characterization of the SARS-CoV-2 Omicron subvariant BA.2.75. Cell Host Microbe (2022).
67. Case, J. B. et al. Neutralizing Antibody and Soluble ACE2 Inhibition of a Replication-Competent VSV-SARS-CoV-2 and a Clinical Isolate of SARS-CoV-2. Cell Host Microbe 28, 475-485 e475 (2020).

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

What is claimed is:

1. A method for assembly of a recombinant viral genome from a plurality of DNA segments, comprising:
a) preparing a series of partially overlapping viral DNA segments designed from a viral genome sequence, wherein each segment comprises different sequences from the viral genome, wherein said overlap comprises unique sequences on their 5′ and 3′ ends;
b) cloning each of said viral DNA segments of a) into a cloning plasmid, said cloning plasmid comprising a cloning site that is flanked on both sides by a Type US restriction endonuclease recognition site or adapters are added to the 5′ and 3′ ends of each viral DNA segment prior to cloning in a cloning plasmid, wherein the adapters comprise the recognition site for a Type IIS restriction endonuclease, said sites positioned to allow removal by digestion with a Type IIS enzyme of a defined number of bases from one strand on both ends of the viral DNA segment;
c) validating the cloned insert segment in each clone of b);
d) digesting the clones of c) with the Type US restriction enzyme, releasing the cloned insert DNA segments, now modified by removal of the defined number of bases from at least one strand at each terminus; and
e) annealing and ligating in a single pot the purified cloned insert DNA segments of d) together into a destination plasmid, whereby an assembled recombinant viral genome with a desired order and orientation of the cloned DNA segments is formed.

2. The method of claim 1, wherein the viral genome is SARS-CoV-2, a variant of SARS-CoV-2, a common cold coronavirus, a variant of a common cold coronavirus, a respiratory syncytial virus, or a variant a respiratory syncytial virus.

3. The method of claim 2, wherein the variant is a naturally occurring variant or genetically/recombinantly engineered variant.

4. The method of claim 3, wherein the naturally occurring variant is Omicron or Delta.

5. The method of claim 1, wherein the purified cloned insert DNA segments that are ligated together in e) come from one virus.

6. The method of claim 1, wherein the purified cloned insert DNA segments that are ligated together in e) come from more than one virus.

7. The method of claim 1, wherein a complete viral genome is formed from the ligated purified cloned insert DNA segments of e).

8. The method of claim 1, wherein when the purified cloned insert DNA segments are ligated together in e), one or more viral open reading frames (ORFs) are absent.

9. The method of claim 8, wherein the absent one or more ORFs is the ORF coding for S, N, M, E viral proteins or combination thereof.

10. The method of claim 9, wherein the absent ORF codes for the S protein.

11. The method of claim 1, wherein a mutation has been entered into one of the viral DNA segments of a).

12. The method of claim 11, wherein the mutation is single point mutation, an addition or a deletion of a nucleotide acid.

13. The method of claim 1, wherein the viral genome is divided into a plurality of DNA segments, wherein there are at least 2 segments.

14. The method of claim 1, wherein the viral genome is divided into a plurality of DNA segments, wherein there are 2, 3, 4, 5, 6, 7, 8, 9, 10, 11, 12, 13, 14, 15, 16, 17, 18, 19, 20 or more segments.

15. The method of claim 1, wherein each of the viral DNA segments of b) are flanked by a Type IIS restriction endonuclease restriction site with opposite orientation.

16. The method of claim 1, wherein the cloning plasmid comprising a cloning site that is flanked on both sides by a Type IIS restriction endonuclease recognition site.

17. The method of claim 1, wherein the Type IIS restriction endonuclease comprises one or more of BbsI, BbvI, BcoDI, BfuAI, BsaI, BsmAI, BsmFI, BspMI, BtgZI, Esp3I, FokI, PaqCI, SfaNI, BaeI, or HgaI.

18. The method of claim 1, wherein the Type IIS restriction endonuclease is BsaI.

19. The method of claim 1, wherein the destination plasmid comprises at least one promotor and Type IIS restriction endonuclease sites.

20. The method of claim 1, wherein the assembled recombinant viral genome of e) is transfected into cells for production of virus.
